SEQUENCE LISTING (214 sequences)

SEQ ID NO: 1 - protein, 415 aa, Trichoderma harzianum
MSPSAISNSPEQRPANNNGTTPDNFAIQPPADFTGYDHVTWWVGNAKQAAAYYTTLFGFETTAYRGLETGSRYFASYVVCNNGVRFVFTSPLRSEAHLPEDETISDSERKLLKEIHAHLERHGDAVKDVAFEVDNVEAVYNKAVAEGAIAVQGPTATKDDHGSVTTAVICTYGDTTHTLINRRGYTGPFLPGFRAGKERTSSVEMPNVPLARIDHCVGNQSWNEMVSACAFYEQCLSFHRFWSVDDSQICTEFSALNSIVMASPNNLVKMPINEPAPGKKKSQIEEYVIFNSGPGVQHIALLTPDIITSVSALRARGVEFINVPTTYYDTMRQRLKTEKRNWQLKEDLDTIQRLNILIDYDEAGYLLQLFTKPLMDRPTVFIEIIQRNNFEGFGAGNFKSLFEAIEREQAERGNL

SEQ ID NO: 2 - DNA, 1248 bp, Trichoderma harzianum
  source 1..1248: mol_type = genomic DNA
atgtccccgtctgctatcagcaactccccagagcagcgacctgcaaacaacaacggcaccacccccgacaacttcgctatccagcctcccgccgacttcaccggctatgaccacgtaacgtggtgggttggcaacgccaagcaggcggccgcttattacaccaccctctttgggttcgagactacggcctatcgtggactcgagactggaagccgatacttcgcttcctatgtcgtctgcaacaatggcgtccgcttcgtcttcacgtcgcctctgcgatcggaggctcacctccctgaagatgagaccatctctgattctgagcggaagctcctgaaggagattcacgctcacctcgagagacacggcgatgccgtcaaggacgttgcctttgaagttgacaacgtcgaggccgtatacaacaaggccgtggctgagggcgccatcgccgtccaaggcccaaccgccaccaaggatgatcacggctccgtcaccacggccgtcatctgcacctatggcgataccacccacactctcatcaaccgccggggctacacgggacctttcctgcccggcttccgcgccggcaaggagcgcacctcgtccgtggagatgcccaacgtgccccttgcccgcatcgaccactgcgtcggcaaccagtcgtggaacgaaatggtctcggcctgcgccttttacgagcagtgcctgtccttccaccgtttctggtccgtcgacgactcccagatctgcaccgagttctcggccctcaactccatcgtcatggcctcgcccaacaacctcgtcaagatgcccatcaacgagcccgccccgggcaagaagaagtcccagatcgaggagtacgtcatcttcaactccggcccgggcgtccagcacatcgccctcctcaccccggacatcatcacctccgtctcggccctccgcgcccgcggcgtcgagttcatcaacgtgcccaccacttactacgacaccatgcgccagcgcctcaagacggagaagcgcaactggcagctcaaggaggacctggacaccatccagcgcctcaacatcctcatcgactacgacgaggccggctacctcctgcagctcttcaccaagccgctcatggaccgccctaccgtcttcattgagattatccagagaaacaactttgagggcttcggcgccggcaacttcaagagcttgttcgaggccattgagcgcgagcaggccgagcgaggaaacctgtaa

SEQ ID NO: 3 - protein, 415 aa, unidentified
  REGION 1..415: note = Talaromyces spp.
MAPSAISDLQSDNLPTTQSALSSYRGYDHVHWYVGNAKQAATFYITRMGFSRVAYRGLETGSRSVCSHVVRNGGITFVLTSPLRSPYNTEKLERLLPSAEEREYLKEIHEHLARHGDAVKDVAFEVDSVDDVFAAAVQNGAVAVSQPKTVEDENGQVRVATIRTYGDTTHTLIQRRGVEKPYSGVFLPGYRDETTSGSSDPITAFLPKVDLRRIDHCVGNQDWDEMEKVCAYYEKVLGFHRFRSVDDKDICTDYSALKSIVMSSPNDIVKMPINEPAHGKKQSQIEEYVDFYDGAGVQHIALLTDDIISAITNLKARGVEFIKVPPTYYDNMWMRLKKAGMMPKEAWEDIKKLDILIDFDEGGYLLQLFTKHLMDRPTVFIEIIQRNNFSGFGAGNFKSLFEAIEREQALRGNLI

SEQ ID NO: 4 - DNA, 1248 bp, unidentified
  misc_feature 1..1248: note = Talaromyces spp.
  source 1..1248: mol_type = genomic DNA
atggcaccatcagccatctcagacctccaatccgacaacctacccacaacccaatccgccctctcctcctaccgcggctacgaccatgtacactggtacgtcggcaacgccaaacaggccgcaaccttctacataacgcgcatgggattttctcgtgtcgcctaccgcggtctcgaaaccggctctcgcagcgtctgctcacacgtcgtgcgcaacggcggtataacttttgtcctgacctcgccgcttcgatcaccctacaacactgagaaactcgagcgcctacttcccagtgctgaagagcgggagtatttgaaagagattcatgagcatttggcacgacatggtgatgcagtcaaagacgtcgcgtttgaggtcgattccgtcgatgatgtgttcgctgctgcggtgcagaatggcgccgttgcggtctcgcaacccaagaccgtggaggatgagaatggtcaagtgagggttgccacgattcggacgtatggggatacgacgcatactttgattcagcgacggggggtcgaaaagccgtattcgggcgttttcttgccagggtacagggatgagacgacttctggtagcagtgatcctatcacggcgttcctgcccaaggttgatttgaggaggattgatcattgtgtggggaatcaggattgggatgaaatggagaaggtctgcgcgtactacgaaaaagtcctcggattccaccgtttccggtccgtagacgacaaagacatctgcacagactactccgccctgaaatcaatcgtcatgtcctcgcccaacgacattgtcaaaatgcccatcaacgaacccgcccacggcaaaaaacaatcccaaatcgaagaatacgtcgacttttacgacggcgccggcgtccaacacattgccctgctgacagacgacataatcagcgcgatcacgaatctcaaagcgcgcggggtggagtttatcaaagtgccgcctacgtattacgataacatgtggatgcggctgaagaaagcgggcatgatgcccaaggaggcgtgggaggatattaagaagttggatattctgatcgattttgatgagggagggtatttgttgcagctcttcacaaagcatctcatggatcggccgactgttttcattgagattattcagcgcaataacttctcaggctttggtgctggtaatttcaagtcgctgttcgaagctattgaacgtgagcaggctcttagaggaaacctgatctga

SEQ ID NO: 5 - protein, 393 aa, Trichoderma harzianum
DNFAIQPPADFTGYDHVTWWVGNAKQAAAYYTTLFGFETTAYRGLETGSRYFASYVVCNNGVRFVFTSPLRSEAHLPEDETISDSERKLLKEIHAHLERHGDAVKDVAFEVDNVEAVYNKAVAEGAIAVQGPTATKDDHGSVTTAVICTYGDTTHTLINRRGYTGPFLPGFRAGKERTSSVEMPNVPLARIDHCVGNQSWNEMVSACAFYEQCLSFHRFWSVDDSQICTEFSALNSIVMASPNNLVKMPINEPAPGKKKSQIEEYVIFNSGPGVQHIALLTPDIITSVSALRARGVEFINVPTTYYDTMRQRLKTEKRNWQLKEDLDTIQRLNILIDYDEAGYLLQLFTKPLMDRPTVFIEIIQRNNFEGFGAGNFKSLFEAIEREQAERGNL

SEQ ID NO: 6 - protein, 377 aa, Trichoderma harzianum
VTWWVGNAKQAAAYYTTLFGFETTAYRGLETGSRYFASYVVCNNGVRFVFTSPLRSEAHLPEDETISDSERKLLKEIHAHLERHGDAVKDVAFEVDNVEAVYNKAVAEGAIAVQGPTATKDDHGSVTTAVICTYGDTTHTLINRRGYTGPFLPGFRAGKERTSSVEMPNVPLARIDHCVGNQSWNEMVSACAFYEQCLSFHRFWSVDDSQICTEFSALNSIVMASPNNLVKMPINEPAPGKKKSQIEEYVIFNSGPGVQHIALLTPDIITSVSALRARGVEFINVPTTYYDTMRQRLKTEKRNWQLKEDLDTIQRLNILIDYDEAGYLLQLFTKPLMDRPTVFIEIIQRNNFEGFGAGNFKSLFEAIEREQAERGNL

SEQ ID NO: 7 - protein, 401 aa, unidentified
  REGION 1..401: note = Talaromyces spp. truncated
PTTQSALSSYRGYDHVHWYVGNAKQAATFYITRMGFSRVAYRGLETGSRSVCSHVVRNGGITFVLTSPLRSPYNTEKLERLLPSAEEREYLKEIHEHLARHGDAVKDVAFEVDSVDDVFAAAVQNGAVAVSQPKTVEDENGQVRVATIRTYGDTTHTLIQRRGVEKPYSGVFLPGYRDETTSGSSDPITAFLPKVDLRRIDHCVGNQDWDEMEKVCAYYEKVLGFHRFRSVDDKDICTDYSALKSIVMSSPNDIVKMPINEPAHGKKQSQIEEYVDFYDGAGVQHIALLTDDIISAITNLKARGVEFIKVPPTYYDNMWMRLKKAGMMPKEAWEDIKKLDILIDFDEGGYLLQLFTKHLMDRPTVFIEIIQRNNFSGFGAGNFKSLFEAIEREQALRGNLI

SEQ ID NO: 8 - protein, 386 aa, unidentified
  REGION 1..386: note = Talaromyces spp. truncated
VHWYVGNAKQAATFYITRMGFSRVAYRGLETGSRSVCSHVVRNGGITFVLTSPLRSPYNTEKLERLLPSAEEREYLKEIHEHLARHGDAVKDVAFEVDSVDDVFAAAVQNGAVAVSQPKTVEDENGQVRVATIRTYGDTTHTLIQRRGVEKPYSGVFLPGYRDETTSGSSDPITAFLPKVDLRRIDHCVGNQDWDEMEKVCAYYEKVLGFHRFRSVDDKDICTDYSALKSIVMSSPNDIVKMPINEPAHGKKQSQIEEYVDFYDGAGVQHIALLTDDIISAITNLKARGVEFIKVPPTYYDNMWMRLKKAGMMPKEAWEDIKKLDILIDFDEGGYLLQLFTKHLMDRPTVFIEIIQRNNFSGFGAGNFKSLFEAIEREQALRGNLI

SEQ ID NO: 9 - protein, 12 aa, Trichoderma harzianum
EAVYNKAVAEGA

SEQ ID NO: 10 - protein, 11 aa, Trichoderma harzianum
VAEGAIAVQGP

SEQ ID NO: 11 - protein, 9 aa, Trichoderma harzianum
FHRFWSVDD

SEQ ID NO: 12 - protein, 10 aa, Trichoderma harzianum
DDSQICTEFS

SEQ ID NO: 13 - protein, 11 aa, Trichoderma harzianum
VEFINVPTTYY

SEQ ID NO: 14 - protein, 12 aa, Trichoderma harzianum
TYYDTMRQRLKT

SEQ ID NO: 15 - protein, 8 aa, Trichoderma harzianum
QRLNILID

SEQ ID NO: 16 - protein, 8 aa, Trichoderma harzianum
IDYDEAGY

SEQ ID NO: 17 - protein, 8 aa, Trichoderma harzianum
EIIQRNNF

SEQ ID NO: 18 - protein, 7 aa, Trichoderma harzianum
NNFEGFG

SEQ ID NO: 19 - protein, 9 aa, Trichoderma harzianum
AVICTYGDT

SEQ ID NO: 20 - protein, 9 aa, Trichoderma harzianum
DTTHTLINR

SEQ ID NO: 21 - protein, 7 aa, Trichoderma harzianum
EMVSACA

SEQ ID NO: 22 - protein, 7 aa, Trichoderma harzianum
CAFYEQC

SEQ ID NO: 23 - protein, 7 aa, Trichoderma harzianum
GFGAGNF

SEQ ID NO: 24 - protein, 6 aa, Trichoderma harzianum
TPDNFA

SEQ ID NO: 25 - protein, 12 aa, unidentified
  REGION 1..12: note = Talaromyces spp
DDVFAAAVQNGA

SEQ ID NO: 26 - protein, 11 aa, unidentified
  REGION 1..11: note = Talaromyces spp
VQNGAVAVSQP

SEQ ID NO: 27 - protein, 9 aa, unidentified
  REGION 1..9: note = Talaromyces spp
FHRFRSVDD

SEQ ID NO: 28 - protein, 10 aa, unidentified
  REGION 1..10: note = Talaromyces spp
DDKDICTDYS

SEQ ID NO: 29 - protein, 11 aa, unidentified
  REGION 1..11: note = Talaromyces spp
VEFIKVPPTYY

SEQ ID NO: 30 - protein, 12 aa, unidentified
  REGION 1..12: note = Talaromyces spp
TYYDNMWMRLKK

SEQ ID NO: 31 - protein, 8 aa, unidentified
  REGION 1..8: note = Talaromyces spp
KKLDILID

SEQ ID NO: 32 - protein, 8 aa, unidentified
  REGION 1..8: note = Talaromyces spp
IDFDEGGY

SEQ ID NO: 33 - protein, 7 aa, unidentified
  REGION 1..7: note = Talaromyces spp
NNFSGFG

SEQ ID NO: 34 - protein, 9 aa, synthetic construct
  REGION 1..9: note = Talaromyces spp
ATIRTYGDT

SEQ ID NO: 35 - protein, 9 aa, unidentified
  REGION 1..9: note = Talaromyces spp
DTTHTLIQR

SEQ ID NO: 36 - protein, 7 aa, unidentified
  REGION 1..7: note = Talaromyces spp
CAYYEKV

SEQ ID NO: 37 - protein, 6 aa, unidentified
  REGION 1..6: note = Talaromyces spp
QSDNLP

SEQ ID NO: 38 - protein, 18 aa, Trichoderma harzianum
EAVYNKAVAEGAIAVQGP

SEQ ID NO: 39 - protein, 17 aa, Trichoderma harzianum
FHRFWSVDDSQICTEFS

SEQ ID NO: 40 - protein, 20 aa, Trichoderma harzianum
VEFINVPTTYYDTMRQRLKT

SEQ ID NO: 41 - protein, 14 aa, Trichoderma harzianum
QRLNILIDYDEAGY

SEQ ID NO: 42 - protein, 12 aa, Trichoderma harzianum
EIIQRNNFEGFG

SEQ ID NO: 43 - protein, 16 aa, Trichoderma harzianum
AVICTYGDTTHTLINR

SEQ ID NO: 44 - protein, 12 aa, Trichoderma harzianum
EMVSACAFYEQC

SEQ ID NO: 45 - protein, 7 aa, Trichoderma harzianum
GFGAGNF

SEQ ID NO: 46 - protein, 6 aa, Trichoderma harzianum
TPDNFA

SEQ ID NO: 47 - protein, 18 aa, unidentified
  REGION 1..18: note = Talaromyces spp
DDVFAAAVQNGAVAVSQP

SEQ ID NO: 48 - protein, 17 aa, unidentified
  REGION 1..17: note = Talaromyces spp
FHRFRSVDDKDICTDYS

SEQ ID NO: 49 - protein, 20 aa, unidentified
  REGION 1..20: note = Talaromyces spp
VEFIKVPPTYYDNMWMRLKK

SEQ ID NO: 50 - protein, 14 aa, unidentified
  REGION 1..14: note = Talaromyces spp
KKLDILIDFDEGGY

SEQ ID NO: 51 - protein, 12 aa, unidentified
  REGION 1..12: note = Talaromyces spp
EIIQRNNFSGFG

SEQ ID NO: 52 - protein, 16 aa, unidentified
  REGION 1..16: note = Talaromyces spp
ATIRTYGDTTHTLIQR

SEQ ID NO: 53 - protein, 12 aa, unidentified
  REGION 1..12: note = Talaromyces spp
EMEKVCAYYEKV

SEQ ID NO: 54 - protein, 6 aa, unidentified
  REGION 1..6: note = Talaromyces spp
QSDNLP

SEQ ID NO: 55 - protein, 12 aa, Glycine max
EAAFSASVAKGA

SEQ ID NO: 56 - protein, 11 aa, Glycine max
VAKGAEPASPP

SEQ ID NO: 57 - protein, 9 aa, Glycine max
FHEFAEFTA

SEQ ID NO: 58 - protein, 10 aa, Glycine max
TAEDVGTSES

SEQ ID NO: 59 - protein, 12 aa, Glycine max
FEFMPSPPPTYY

SEQ ID NO: 60 - protein, 10 aa, Glycine max
TYYANLHNRA

SEQ ID NO: 61 - protein, 8 aa, Glycine max
EELGILVD

SEQ ID NO: 62 - protein, 8 aa, Glycine max
VDRDDQGT

SEQ ID NO: 63 - protein, 9 aa, Glycine max
EIIQRIGCM

SEQ ID NO: 64 - protein, 9 aa, Glycine max
CMVEDEEGK

SEQ ID NO: 65 - protein, 8 aa, Glycine max
GKVYQKGA

SEQ ID NO: 66 - protein, 10 aa, Glycine max
YQKGACGGFG

SEQ ID NO: 67 - protein, 9 aa, Glycine max
AEVRLYGDV

SEQ ID NO: 68 - protein, 9 aa, Glycine max
DVVLRYVSY

SEQ ID NO: 69 - protein, 7 aa, Glycine max
ELAPAVR

SEQ ID NO: 70 - protein, 7 aa, Glycine max
VRYLKGF

SEQ ID NO: 71 - protein, 7 aa, Glycine max
GFGKGNF

SEQ ID NO: 72 - protein, 6 aa, Glycine max
FVRTNP

SEQ ID NO: 73 - protein, 12 aa, synthetic construct
  REGION 1..12: note = Dicot plant HPPD consensus sequence
  SITE 1: note = X is E or R
  SITE 2: note = X is any amino acid
  SITE 5: note = X is S or T
  SITE 6: note = X is A, I or T
  SITE 9: note = X is any amino acid
  SITE 10: note = X is K, N or H
XXAFXXSVXXGA

SEQ ID NO: 74 - protein, 11 aa, synthetic construct
  REGION 1..11: note = Dicot plant HPPD consensus sequence
  SITE 2: note = MISC_FEATURE - X = any amino acid
  SITE 3: note = MISC_FEATURE - X = K, N or H
  SITE 8: note = MISC_FEATURE - X = A or S
  SITE 10: note = MISC_FEATURE - X = P or E
VXXGAEPXSXP

SEQ ID NO: 75 - protein, 9 aa, synthetic construct
  REGION 1..9: note = Dicot plant HPPD consensus sequence
  SITE 3: note = MISC_FEATURE - X = E or Q
  SITE 9: note = MISC_FEATURE - X = A or T
FHXFAEFTX

SEQ ID NO: 76 - protein, 10 aa, synthetic construct
  REGION 1..10: note = Dicot plant HPPD consensus sequence
  SITE 2: note = MISC_FEATURE - X = A or T
  SITE 3: note = MISC_FEATURE - X = E or D
  SITE 8: note = MISC_FEATURE - X = A or S
TXXDVGTXES

SEQ ID NO: 77 - protein, 12 aa, synthetic construct
  REGION 1..12: note = Dicot plant HPPD consensus sequence
  SITE 2: note = MISC_FEATURE - X = E or D
  SITE 9: note = MISC_FEATURE - X = P or V
FXFMPSPPXTYY

SEQ ID NO: 78 - protein, 10 aa, synthetic construct
  REGION 1..10: note = Dicot plant HPPD consensus sequence
  SITE 4: note = MISC_FEATURE - X = any amino acid
  SITE 5: note = MISC_FEATURE - X = N or K
  SITE 7: note = MISC_FEATURE - X = H, K or R
  SITE 8: note = MISC_FEATURE - X = any amino acid
  SITE 9: note = MISC_FEATURE - X = A or G
TYYXXLXXXL

SEQ ID NO: 79 - protein, 8 aa, synthetic construct
  REGION 1..8: note = Dicot plant HPPD consensus sequence
  SITE 3: note = MISC_FEATURE - X = R or K
VDXDDQGT

SEQ ID NO: 80 - protein, 9 aa, synthetic construct
  REGION 1..9: note = Dicot plant HPPD consensus sequence
  SITE 6: note = MISC_FEATURE - X = I or V
EIIQRXGCM

SEQ ID NO: 81 - protein, 9 aa, synthetic construct
  REGION 1..9: note = Dicot plant HPPD consensus sequence
  SITE 3: note = MISC_FEATURE - X = any amino acid
  SITE 4: note = MISC_FEATURE - X = E, K or Q
  SITE 5: note = MISC_FEATURE - X = D or N
  SITE 6: note = MISC_FEATURE - X = E or A
  SITE 9: note = MISC_FEATURE - X = K or E
CMXXXXEGX

SEQ ID NO: 82 - protein, 8 aa, synthetic construct
  REGION 1..8: note = Dicot plant HPPD consensus sequence
  SITE 2: note = MISC_FEATURE - X = K or E
  SITE 3: note = MISC_FEATURE - X = X = any amino acid
  SITE 6: note = MISC_FEATURE - X = K or S
  SITE 8: note = MISC_FEATURE - X = G or A
GXXYQXGX

SEQ ID NO: 83 - protein, 10 aa, synthetic construct
  REGION 1..10: note = Dicot plant HPPD consensus sequence
  SITE 3: note = MISC_FEATURE - X = K or S
  SITE 5: note = MISC_FEATURE - X = G or A
YQXGXCGGFG

SEQ ID NO: 84 - protein, 9 aa, synthetic construct
  REGION 1..9: note = Dicot plant HPPD consensus sequence
  SITE 1: note = MISC_FEATURE - X = A or S
  SITE 4: note = MISC_FEATURE - X = R, H or K
XEVXLYGDV

SEQ ID NO: 85-86 - skipped (2 entries)

SEQ ID NO: 87 - protein, 6 aa, synthetic construct
  REGION 1..6: note = Dicot plant HPPD consensus sequence
  SITE 2: note = MISC_FEATURE - X = V or I
  SITE 4: note = MISC_FEATURE - X = any amino acid
FXRXNP

SEQ ID NO: 88 - protein, 12 aa, synthetic construct
  REGION 1..12: note = Monocot plant HPPD consensus sequence
  SITE 1: note = MISC_FEATURE - X = E or A
  SITE 2: note = MISC_FEATURE - X = D or E
  SITE 6: note = MISC_FEATURE - X = A or V
  SITE 10: note = MISC_FEATURE - X = A or G
XXAFRXSVAXGA

SEQ ID NO: 89 - protein, 11 aa, synthetic construct
  REGION 1..11: note = Monocot plant HPPD consensus sequence
  SITE 3: note = MISC_FEATURE - X = A or G
  SITE 10: note = MISC_FEATURE - X = Q or A
VAXGARPAFXP

SEQ ID NO: 90 - protein, 9 aa, synthetic construct
  REGION 1..9: note = Monocot plant HPPD consensus sequence
  SITE 9: note = MISC_FEATURE - X = A or T
FHEFAEFTX

SEQ ID NO: 91 - protein, 10 aa, synthetic construct
  REGION 1..10: note = Monocot plant HPPD consensus sequence
  SITE 2: note = MISC_FEATURE - X = A or T
  SITE 8: note = MISC_FEATURE - X = A or T
TXEDVGTXES

SEQ ID NO: 92 - protein, 13 aa, synthetic construct
  REGION 1..13: note = Monocot plant HPPD consensus sequence
  SITE 4: note = MISC_FEATURE - X = L or M
  SITE 8: note = MISC_FEATURE - X = P, T or Q
  SITE 9: note = MISC_FEATURE - X = S, P or A
  SITE 10: note = MISC_FEATURE - X = D, N or K
FEFXAPPXXXYYW

SEQ ID NO: 93 - protein, 10 aa, synthetic construct
  REGION 1..10: note = Monocot plant HPPD consensus sequence
  SITE 1: note = MISC_FEATURE - X = D, N or K
  SITE 4: note = MISC_FEATURE - X = D or E
XYYXGVRRGV

SEQ ID NO: 94 - protein, 8 aa, synthetic construct
  REGION 1..8: note = Monocot plant HPPD consensus sequence
QELGVLVD

SEQ ID NO: 95 - protein, 8 aa, synthetic construct
  REGION 1..8: note = Monocot plant HPPD consensus sequence
VDRDDQGV

SEQ ID NO: 96 - protein, 9 aa, synthetic construct
  REGION 1..9: note = Monocot plant HPPD consensus sequence
EMIQRIGCM

SEQ ID NO: 97 - protein, 9 aa, synthetic construct
  REGION 1..9: note = Monocot plant HPPD consensus sequence
  SITE 7: note = MISC_FEATURE - X = K, S or V
CMEKDEXGQ

SEQ ID NO: 98 - protein, 8 aa, synthetic construct
  REGION 1..8: note = Monocot plant HPPD consensus sequence
GQEYQKGA

SEQ ID NO: 99 - protein, 9 aa, synthetic construct
  REGION 1..9: note = AEVELYGDV
AEVELYGDV

SEQ ID NO: 100-101 - skipped (2 entries)

SEQ ID NO: 102 - protein, 6 aa, synthetic construct
  REGION 1..6: note = Monocot plant HPPD consensus sequence
  SITE 1: note = MISC_FEATURE - X = F or V
  SITE 4: note = MISC_FEATURE - X = F, A or V
XVRXNP

SEQ ID NO: 103 - protein, 28 aa, Arabidopsis thaliana
MPPTPTTAAATGAGAAAVTPEHAAFRLV

SEQ ID NO: 104 - protein, 22 aa, Arabidopsis thaliana
MGHQNAAVSENQNHDDGAASSP

SEQ ID NO: 105 - protein, 49 aa, Arabidopsis thaliana
MGHQNAAVSENQNHDDGAASSPGFKLVGFSKFVRKNPKSDKFKVKRFHH

SEQ ID NO: 106 - protein, 415 aa, synthetic construct
  REGION 1..415: note = Trichoderma HPPD wherein aa 1-22 are substituted w aa 1-22 of Arabidopsis thaliana
MGHQNAAVSENQNHDDGAASSPDNFAIQPPADFTGYDHVTWWVGNAKQAAAYYTTLFGFETTAYRGLETGSRYFASYVVCNNGVRFVFTSPLRSEAHLPEDETISDSERKLLKEIHAHLERHGDAVKDVAFEVDNVEAVYNKAVAEGAIAVQGPTATKDDHGSVTTAVICTYGDTTHTLINRRGYTGPFLPGFRAGKERTSSVEMPNVPLARIDHCVGNQSWNEMVSACAFYEQCLSFHRFWSVDDSQICTEFSALNSIVMASPNNLVKMPINEPAPGKKKSQIEEYVIFNSGPGVQHIALLTPDIITSVSALRARGVEFINVPTTYYDTMRQRLKTEKRNWQLKEDLDTIQRLNILIDYDEAGYLLQLFTKPLMDRPTVFIEIIQRNNFEGFGAGNFKSLFEAIEREQAERGNL

SEQ ID NO: 107 - protein, 442 aa, synthetic construct
  REGION 1..442: note = Trichoderma HPPD wherein aa 1-22 are substituted w aa 1-49 of Arabidopsis thaliana
MGHQNAAVSENQNHDDGAASSPGFKLVGFSKFVRKNPKSDKFKVKRFHHDNFAIQPPADFTGYDHVTWWVGNAKQAAAYYTTLFGFETTAYRGLETGSRYFASYVVCNNGVRFVFTSPLRSEAHLPEDETISDSERKLLKEIHAHLERHGDAVKDVAFEVDNVEAVYNKAVAEGAIAVQGPTATKDDHGSVTTAVICTYGDTTHTLINRRGYTGPFLPGFRAGKERTSSVEMPNVPLARIDHCVGNQSWNEMVSACAFYEQCLSFHRFWSVDDSQICTEFSALNSIVMASPNNLVKMPINEPAPGKKKSQIEEYVIFNSGPGVQHIALLTPDIITSVSALRARGVEFINVPTTYYDTMRQRLKTEKRNWQLKEDLDTIQRLNILIDYDEAGYLLQLFTKPLMDRPTVFIEIIQRNNFEGFGAGNFKSLFEAIEREQAERGNL

SEQ ID NO: 108 - protein, 399 aa, synthetic construct
  REGION 1..399: note = Trichoderma HPPD wherein aa 1-38 are substituted w aa 1-22 of Arabidopsis thaliana
MGHQNAAVSENQNHDDGAASSPVTWWVGNAKQAAAYYTTLFGFETTAYRGLETGSRYFASYVVCNNGVRFVFTSPLRSEAHLPEDETISDSERKLLKEIHAHLERHGDAVKDVAFEVDNVEAVYNKAVAEGAIAVQGPTATKDDHGSVTTAVICTYGDTTHTLINRRGYTGPFLPGFRAGKERTSSVEMPNVPLARIDHCVGNQSWNEMVSACAFYEQCLSFHRFWSVDDSQICTEFSALNSIVMASPNNLVKMPINEPAPGKKKSQIEEYVIFNSGPGVQHIALLTPDIITSVSALRARGVEFINVPTTYYDTMRQRLKTEKRNWQLKEDLDTIQRLNILIDYDEAGYLLQLFTKPLMDRPTVFIEIIQRNNFEGFGAGNFKSLFEAIEREQAERGNL

SEQ ID NO: 109 - protein, 426 aa, synthetic construct
  REGION 1..426: note = Trichoderma HPPD wherein aa 1-38 are substituted w aa 1-49 of Arabidopsis thaliana
MGHQNAAVSENQNHDDGAASSPGFKLVGFSKFVRKNPKSDKFKVKRFHHVTWWVGNAKQAAAYYTTLFGFETTAYRGLETGSRYFASYVVCNNGVRFVFTSPLRSEAHLPEDETISDSERKLLKEIHAHLERHGDAVKDVAFEVDNVEAVYNKAVAEGAIAVQGPTATKDDHGSVTTAVICTYGDTTHTLINRRGYTGPFLPGFRAGKERTSSVEMPNVPLARIDHCVGNQSWNEMVSACAFYEQCLSFHRFWSVDDSQICTEFSALNSIVMASPNNLVKMPINEPAPGKKKSQIEEYVIFNSGPGVQHIALLTPDIITSVSALRARGVEFINVPTTYYDTMRQRLKTEKRNWQLKEDLDTIQRLNILIDYDEAGYLLQLFTKPLMDRPTVFIEIIQRNNFEGFGAGNFKSLFEAIEREQAERGNL

SEQ ID NO: 110 - protein, 423 aa, synthetic construct
  REGION 1..423: note = Talaromyces HPPD wherein aa 1-22 are substituted w aa 1-22 of Arabidopsis thaliana
MGHQNAAVSENQNHDDGAASSPPTTQSALSSYRGYDHVHWYVGNAKQAATFYITRMGFSRVAYRGLETGSRSVCSHVVRNGGITFVLTSPLRSPYNTEKLERLLPSAEEREYLKEIHEHLARHGDAVKDVAFEVDSVDDVFAAAVQNGAVAVSQPKTVEDENGQVRVATIRTYGDTTHTLIQRRGVEKPYSGVFLPGYRDETTSGSSDPITAFLPKVDLRRIDHCVGNQDWDEMEKVCAYYEKVLGFHRFRSVDDKDICTDYSALKSIVMSSPNDIVKMPINEPAHGKKQSQIEEYVDFYDGAGVQHIALLTDDIISAITNLKARGVEFIKVPPTYYDNMWMRLKKAGMMPKEAWEDIKKLDILIDFDEGGYLLQLFTKHLMDRPTVFIEIIQRNNFSGFGAGNFKSLFEAIEREQALRGNLI

SEQ ID NO: 111 - protein, 450 aa, synthetic construct
  REGION 1..450: note = Talaromyces HPPD wherein aa 1-22 are substituted w aa 1-49 of Arabidopsis thaliana
MGHQNAAVSENQNHDDGAASSPGFKLVGFSKFVRKNPKSDKFKVKRFHHPTTQSALSSYRGYDHVHWYVGNAKQAATFYITRMGFSRVAYRGLETGSRSVCSHVVRNGGITFVLTSPLRSPYNTEKLERLLPSAEEREYLKEIHEHLARHGDAVKDVAFEVDSVDDVFAAAVQNGAVAVSQPKTVEDENGQVRVATIRTYGDTTHTLIQRRGVEKPYSGVFLPGYRDETTSGSSDPITAFLPKVDLRRIDHCVGNQDWDEMEKVCAYYEKVLGFHRFRSVDDKDICTDYSALKSIVMSSPNDIVKMPINEPAHGKKQSQIEEYVDFYDGAGVQHIALLTDDIISAITNLKARGVEFIKVPPTYYDNMWMRLKKAGMMPKEAWEDIKKLDILIDFDEGGYLLQLFTKHLMDRPTVFIEIIQRNNFSGFGAGNFKSLFEAIEREQALRGNLI

SEQ ID NO: 112 - protein, 408 aa, synthetic construct
  REGION 1..408: note = Talaromyces HPPD wherein aa 1-30 are substituted w aa 1-22 of Arabidopsis thaliana
MGHQNAAVSENQNHDDGAASSPVHWYVGNAKQAATFYITRMGFSRVAYRGLETGSRSVCSHVVRNGGITFVLTSPLRSPYNTEKLERLLPSAEEREYLKEIHEHLARHGDAVKDVAFEVDSVDDVFAAAVQNGAVAVSQPKTVEDENGQVRVATIRTYGDTTHTLIQRRGVEKPYSGVFLPGYRDETTSGSSDPITAFLPKVDLRRIDHCVGNQDWDEMEKVCAYYEKVLGFHRFRSVDDKDICTDYSALKSIVMSSPNDIVKMPINEPAHGKKQSQIEEYVDFYDGAGVQHIALLTDDIISAITNLKARGVEFIKVPPTYYDNMWMRLKKAGMMPKEAWEDIKKLDILIDFDEGGYLLQLFTKHLMDRPTVFIEIIQRNNFSGFGAGNFKSLFEAIEREQALRGNLI

SEQ ID NO: 113 - protein, 435 aa, synthetic construct
  REGION 1..435: note = Talaromyces HPPD wherein aa 1-30 are substituted w aa 1-49 of Arabidopsis thaliana
MGHQNAAVSENQNHDDGAASSPGFKLVGFSKFVRKNPKSDKFKVKRFHHVHWYVGNAKQAATFYITRMGFSRVAYRGLETGSRSVCSHVVRNGGITFVLTSPLRSPYNTEKLERLLPSAEEREYLKEIHEHLARHGDAVKDVAFEVDSVDDVFAAAVQNGAVAVSQPKTVEDENGQVRVATIRTYGDTTHTLIQRRGVEKPYSGVFLPGYRDETTSGSSDPITAFLPKVDLRRIDHCVGNQDWDEMEKVCAYYEKVLGFHRFRSVDDKDICTDYSALKSIVMSSPNDIVKMPINEPAHGKKQSQIEEYVDFYDGAGVQHIALLTDDIISAITNLKARGVEFIKVPPTYYDNMWMRLKKAGMMPKEAWEDIKKLDILIDFDEGGYLLQLFTKHLMDRPTVFIEIIQRNNFSGFGAGNFKSLFEAIEREQALRGNLI

SEQ ID NO: 114 - protein, 437 aa, synthetic construct
  REGION 1..437: note = Trichoderma HPPD + aa 1-22 of Arabidopsis thaliana at N'
MGHQNAAVSENQNHDDGAASSPMSPSAISNSPEQRPANNNGTTPDNFAIQPPADFTGYDHVTWWVGNAKQAAAYYTTLFGFETTAYRGLETGSRYFASYVVCNNGVRFVFTSPLRSEAHLPEDETISDSERKLLKEIHAHLERHGDAVKDVAFEVDNVEAVYNKAVAEGAIAVQGPTATKDDHGSVTTAVICTYGDTTHTLINRRGYTGPFLPGFRAGKERTSSVEMPNVPLARIDHCVGNQSWNEMVSACAFYEQCLSFHRFWSVDDSQICTEFSALNSIVMASPNNLVKMPINEPAPGKKKSQIEEYVIFNSGPGVQHIALLTPDIITSVSALRARGVEFINVPTTYYDTMRQRLKTEKRNWQLKEDLDTIQRLNILIDYDEAGYLLQLFTKPLMDRPTVFIEIIQRNNFEGFGAGNFKSLFEAIEREQAERGNL

SEQ ID NO: 115 - protein, 464 aa, synthetic construct
  REGION 1..464: note = Trichoderma HPPD + aa 1-49 of Arabidopsis thaliana at N'
MGHQNAAVSENQNHDDGAASSPGFKLVGFSKFVRKNPKSDKFKVKRFHHMSPSAISNSPEQRPANNNGTTPDNFAIQPPADFTGYDHVTWWVGNAKQAAAYYTTLFGFETTAYRGLETGSRYFASYVVCNNGVRFVFTSPLRSEAHLPEDETISDSERKLLKEIHAHLERHGDAVKDVAFEVDNVEAVYNKAVAEGAIAVQGPTATKDDHGSVTTAVICTYGDTTHTLINRRGYTGPFLPGFRAGKERTSSVEMPNVPLARIDHCVGNQSWNEMVSACAFYEQCLSFHRFWSVDDSQICTEFSALNSIVMASPNNLVKMPINEPAPGKKKSQIEEYVIFNSGPGVQHIALLTPDIITSVSALRARGVEFINVPTTYYDTMRQRLKTEKRNWQLKEDLDTIQRLNILIDYDEAGYLLQLFTKPLMDRPTVFIEIIQRNNFEGFGAGNFKSLFEAIEREQAERGNL

SEQ ID NO: 116 - protein, 437 aa, synthetic construct
  REGION 1..437: note = Talaromyces HPPD + aa 1-22 of Arabidopsis thaliana at N'
MGHQNAAVSENQNHDDGAASSPMAPSAISDLQSDNLPTTQSALSSYRGYDHVHWYVGNAKQAATFYITRMGFSRVAYRGLETGSRSVCSHVVRNGGITFVLTSPLRSPYNTEKLERLLPSAEEREYLKEIHEHLARHGDAVKDVAFEVDSVDDVFAAAVQNGAVAVSQPKTVEDENGQVRVATIRTYGDTTHTLIQRRGVEKPYSGVFLPGYRDETTSGSSDPITAFLPKVDLRRIDHCVGNQDWDEMEKVCAYYEKVLGFHRFRSVDDKDICTDYSALKSIVMSSPNDIVKMPINEPAHGKKQSQIEEYVDFYDGAGVQHIALLTDDIISAITNLKARGVEFIKVPPTYYDNMWMRLKKAGMMPKEAWEDIKKLDILIDFDEGGYLLQLFTKHLMDRPTVFIEIIQRNNFSGFGAGNFKSLFEAIEREQALRGNLI

SEQ ID NO: 117 - protein, 464 aa, synthetic construct
  REGION 1..464: note = Talaromyces HPPD + aa 1-49 of Arabidopsis thaliana at N'
MGHQNAAVSENQNHDDGAASSPGFKLVGFSKFVRKNPKSDKFKVKRFHHMAPSAISDLQSDNLPTTQSALSSYRGYDHVHWYVGNAKQAATFYITRMGFSRVAYRGLETGSRSVCSHVVRNGGITFVLTSPLRSPYNTEKLERLLPSAEEREYLKEIHEHLARHGDAVKDVAFEVDSVDDVFAAAVQNGAVAVSQPKTVEDENGQVRVATIRTYGDTTHTLIQRRGVEKPYSGVFLPGYRDETTSGSSDPITAFLPKVDLRRIDHCVGNQDWDEMEKVCAYYEKVLGFHRFRSVDDKDICTDYSALKSIVMSSPNDIVKMPINEPAHGKKQSQIEEYVDFYDGAGVQHIALLTDDIISAITNLKARGVEFIKVPPTYYDNMWMRLKKAGMMPKEAWEDIKKLDILIDFDEGGYLLQLFTKHLMDRPTVFIEIIQRNNFSGFGAGNFKSLFEAIEREQALRGNLI

SEQ ID NO: 118 - protein, 446 aa, Glycine max
  SITE 396: note = MISC_FEATURE - X = any amino acid
MPIPCNEIQAQAQAQAQPGFKLVGFKNFVRTNPKSDRFQVNRFHHIEFWCTDATNASRRFSWGLGMPIVAKSDLSTGNQIHASYLLRSGDLSFLFSAPYSPSLSAGSSAASSASIPSFDAATCLAFAAKHGFGVRAIALEVADAEAAFSASVAKGAEPASPPVLVDDRTGFAEVRLYGDVVLRYVSYKDAAPQAPHADPSRWFLPGFEAAASSSSFPELDYGIRRLDHAVGNVPELAPAVRYLKGFSGFHEFAEFTAEDVGTSESGLNSVVLANNSETVLLPLNEPVYGTKRKSQIETYLEHNEGAGVQHLALVTHDIFTTLREMRKRSFLGGFEFMPSPPPTYYANLHNRAADVLTVDQIKQCEELGILVDRDDQGTLLQIFTKPVGDRPTIFIXIIQRIGCMVEDEEGKVYQKGACGGFGKGNFSELFKSIEEYEKTLEAKRTA

SEQ ID NO: 119 - protein, 12 aa, synthetic construct
  REGION 1..12: note = Dicot plant HPPD modified consensus sequence
  SITE 1: note = MISC_FEATURE - X= E or R
  SITE 2: note = MISC_FEATURE - X= any amino acid
  SITE 5: note = MISC_FEATURE - X= S or T
  SITE 6: note = MISC_FEATURE - X= A, I or T
  SITE 9: note = MISC_FEATURE - X= any amino acid
  SITE 10: note = MISC_FEATURE - X= K, N or H
XXAYXXSVXXGA

SEQ ID NO: 120 - protein, 12 aa, synthetic construct
  REGION 1..12: note = Dicot plant HPPD modified consensus sequence
  SITE 1: note = MISC_FEATURE - X = E or R
  SITE 2: note = MISC_FEATURE - X = any amino acid
  SITE 6: note = MISC_FEATURE - X = A, I or T
  SITE 9: note = MISC_FEATURE - X = any amino acid
  SITE 10: note = MISC_FEATURE - X = K, N or H
XXAFNXSVXXGA

SEQ ID NO: 121 - protein, 12 aa, synthetic construct
  REGION 1..12: note = Dicot plant HPPD modified consensus sequence
  SITE 1: note = MISC_FEATURE - X= E or R
  SITE 2: note = MISC_FEATURE - X= any amino acid
  SITE 5: note = MISC_FEATURE - X= S or T
  SITE 9: note = MISC_FEATURE - X= any amino acid
  SITE 10: note = MISC_FEATURE - X= K, N, H
XXAFXKSVXXGA

SEQ ID NO: 122 - protein, 12 aa, synthetic construct
  REGION 1..12: note = Dicot plant HPPD modified consensus sequence
  SITE 1: note = MISC_FEATURE - X = E or R
  SITE 2: note = MISC_FEATURE - X = any amino acid
  SITE 5: note = MISC_FEATURE - X = S or T
  SITE 6: note = MISC_FEATURE - X = A, I or T
  SITE 9: note = MISC_FEATURE - X = any amino acid
  SITE 10: note = MISC_FEATURE - X = K, N or H
XXAFXXAVXXGA

SEQ ID NO: 123 - protein, 12 aa, synthetic construct
  REGION 1..12: note = Dicot plant HPPD modified consensus sequence
  SITE 1: note = MISC_FEATURE - X = E or R
  SITE 2: note = MISC_FEATURE - X = any amino acid
  SITE 5: note = MISC_FEATURE - X = S or T
  SITE 6: note = MISC_FEATURE - X = A, I or T
  SITE 9: note = MISC_FEATURE - X = any amino acid
XXAFXXSVXEGA

SEQ ID NO: 124 - protein, 11 aa, synthetic construct
  REGION 1..11: note = Dicot plant HPPD modified consensus sequence
  SITE 2: note = MISC_FEATURE - X = any amino acid
  SITE 8: note = MISC_FEATURE - X = A or S
  SITE 10: note = MISC_FEATURE - X = P or E
VXEGAEPXSXP

SEQ ID NO: 125 - protein, 11 aa, synthetic construct
  REGION 1..11: note = Dicot plant HPPD modified consensus sequence
  SITE 2: note = MISC_FEATURE - X = any amino acid
  SITE 3: note = MISC_FEATURE - X = K, N or H
  SITE 8: note = MISC_FEATURE - X = A or S
  SITE 10: note = MISC_FEATURE - X = P or E
VXXGAIPXSXP

SEQ ID NO: 126 - protein, 11 aa, synthetic construct
  REGION 1..11: note = Dicot plant HPPD modified consensus sequence
  SITE 2: note = MISC_FEATURE - X = any amino acid
  SITE 3: note = MISC_FEATURE - X = K, N or H
  SITE 8: note = MISC_FEATURE - X = A or S
  SITE 10: note = MISC_FEATURE - X = P or E
VXXGAEAXSXP

SEQ ID NO: 127 - protein, 11 aa, synthetic construct
  REGION 1..11: note = Dicot plant HPPD modified consensus sequence
  SITE 2: note = MISC_FEATURE - X = any amino acid
  SITE 3: note = MISC_FEATURE - X = K, N or H
  SITE 8: note = MISC_FEATURE - X = A or S
  SITE 10: note = MISC_FEATURE - X = P or E
VXXGAEPXQXP

SEQ ID NO: 128 - protein, 11 aa, synthetic construct
  REGION 1..11: note = Dicot plant HPPD modified consensus sequence
  SITE 2: note = MISC_FEATURE - X = any amino acid
  SITE 3: note = MISC_FEATURE - X = K, N or H
  SITE 8: note = MISC_FEATURE - X = A or S
VXXGAEPXSGP

SEQ ID NO: 129 - protein, 9 aa, synthetic construct
  REGION 1..9: note = Dicot plant HPPD modified consensus sequence
  SITE 3: note = MISC_FEATURE - X = E or Q
  SITE 5: note = MISC_FEATURE - X = W or R
  SITE 9: note = MISC_FEATURE - X = A or T
FHXFXEFTX

SEQ ID NO: 130 - protein, 9 aa, synthetic construct
  REGION 1..9: note = Dicot plant HPPD modified consensus sequence
  SITE 3: note = MISC_FEATURE - X = E or Q
  SITE 9: note = MISC_FEATURE - X = A or T
FHXFAEFDX

SEQ ID NO: 131 - protein, 9 aa, synthetic construct
  REGION 1..9: note = Dicot plant HPPD modified consensus sequence
  SITE 3: note = MISC_FEATURE - X = E or Q
FHXFAEFTD

SEQ ID NO: 132 - protein, 10 aa, synthetic construct
  REGION 1..10: note = DXXDVGTXES
  SITE 2: note = MISC_FEATURE - X = A or T
  SITE 3: note = MISC_FEATURE - X = E or D
  SITE 8: note = MISC_FEATURE - X = A or S
DXXDVGTXES

SEQ ID NO: 133 - protein, 10 aa, synthetic construct
  REGION 1..10: note = Dicot plant HPPD modified consensus sequence
  SITE 3: note = MISC_FEATURE - X = E or D
  SITE 8: note = MISC_FEATURE - X = A or S
TDXDVGTXES

SEQ ID NO: 134 - protein, 10 aa, synthetic construct
  REGION 1..10: note = Dicot plant HPPD modified consensus sequence
  SITE 2: note = MISC_FEATURE - X = A or T
  SITE 3: note = MISC_FEATURE - X = E or D
  SITE 8: note = MISC_FEATURE - X = A or S
  SITE 9: note = MISC_FEATURE - X = F or Y
TXXDVGTXXS

SEQ ID NO: 135 - protein, 12 aa, synthetic construct
  REGION 1..12: note = Dicot plant HPPD modified consensus sequence
  SITE 2: note = MISC_FEATURE - X = E or D
  SITE 9: note = MISC_FEATURE - X = P or V
FXFIPSPPXTYY

SEQ ID NO: 136 - protein, 12 aa, synthetic construct
  REGION 1..12: note = Dicot plant HPPD modified consensus sequence
  SITE 2: note = MISC_FEATURE - X = E or D
  SITE 9: note = MISC_FEATURE - X = P or V
FXFMNSPPXTYY

SEQ ID NO: 137 - protein, 12 aa, synthetic construct
  REGION 1..12: note = Dicot plant HPPD modified consensus sequence
  SITE 2: note = MISC_FEATURE - X = E or D
  SITE 9: note = MISC_FEATURE - X = P or V
FXFMPVPPXTYY

SEQ ID NO: 138 - protein, 12 aa, synthetic construct
  REGION 1..12: note = Dicot plant HPPD modified consensus sequence
  SITE 2: note = MISC_FEATURE - X = E or D
  SITE 8: note = MISC_FEATURE - X = G or T
  SITE 9: note = MISC_FEATURE - X = P or V
FXFMPSPXXTYY

SEQ ID NO: 139 - protein, 12 aa, synthetic construct
  REGION 1..12: note = Dicot plant HPPD modified consensus sequence
  SITE 2: note = MISC_FEATURE - X = E or D
FXFMPSPPGTYY

SEQ ID NO: 140 - protein, 11 aa, synthetic construct
  REGION 1..11: note = Dicot plant HPPD modified consensus sequence
  SITE 2: note = MISC_FEATURE - X = E or D
FXFMPSPPTYY

SEQ ID NO: 141 - protein, 10 aa, synthetic construct
  REGION 1..10: note = Dicot plant HPPD modified consensus sequence
  SITE 5: note = MISC_FEATURE - X = N or K
  SITE 7: note = MISC_FEATURE - X = H, K or R
  SITE 8: note = MISC_FEATURE - X = any amino acid
  SITE 9: note = MISC_FEATURE - X = A or G
TYYDXLXXXL

SEQ ID NO: 142 - protein, 10 aa, synthetic construct
  REGION 1..10: note = Dicot plant HPPD modified consensus sequence
  SITE 4: note = MISC_FEATURE - X = any amino acid
  SITE 5: note = MISC_FEATURE - X = N or K
  SITE 7: note = MISC_FEATURE - X = H, K or R
  SITE 9: note = MISC_FEATURE - X = A or G
TYYXXLXLXL

SEQ ID NO: 143 - protein, 8 aa, synthetic construct
  REGION 1..8: note = Dicot plant HPPD modified consensus sequence
  SITE 3: note = MISC_FEATURE - X = Fo r Y
VDXDDQGT

SEQ ID NO: 144 - protein, 8 aa, synthetic construct
  REGION 1..8: note = Dicot plant HPPD modified consensus sequence
  SITE 3: note = MISC_FEATURE - X = R or K
  SITE 6: note = MISC_FEATURE - X = A or G
VDXDDXGT

SEQ ID NO: 145 - protein, 9 aa, synthetic construct
  REGION 1..9: note = Dicot plant HPPD modified consensus sequence
EIIQRNGCM

SEQ ID NO: 146 - protein, 9 aa, synthetic construct
  REGION 1..9: note = Dicot plant HPPD modified consensus sequence
  SITE 6: note = MISC_FEATURE - X = I or V
EIIQRXNCM

SEQ ID NO: 147 - protein, 9 aa, synthetic construct
  REGION 1..9: note = Dicot plant HPPD modified consensus sequence
  SITE 6: note = MISC_FEATURE - X = I or V
EIIQRXGFM

SEQ ID NO: 148 - protein, 9 aa, synthetic construct
  REGION 1..9: note = Dicot plant HPPD modified consensus sequence
  SITE 6: note = MISC_FEATURE - X = I or V
EIIQRXGCG

SEQ ID NO: 149 - protein, 8 aa, synthetic construct
  REGION 1..8: note = Dicot plant HPPD modified consensus sequence
  SITE 6: note = MISC_FEATURE - X = I or V
EIIQRXGC

SEQ ID NO: 150 - protein, 9 aa, synthetic construct
  REGION 1..9: note = Dicot plant HPPD modified consensus sequence
  SITE 3: note = MISC_FEATURE - X = any amino acid
  SITE 4: note = MISC_FEATURE - X = E, K or Q
  SITE 5: note = MISC_FEATURE - X = X = D or N
  SITE 6: note = MISC_FEATURE - X = E or A
  SITE 9: note = MISC_FEATURE - X = K or E
FMXXXXEGX

SEQ ID NO: 151 - protein, 10 aa, synthetic construct
  REGION 1..10: note = Dicot plant HPPD modified consensus sequence
  SITE 3: note = MISC_FEATURE - X = K or S
  SITE 5: note = MISC_FEATURE - X = G or A
  SITE 7: note = MISC_FEATURE - X = E or S
YQXGXCXGFG

SEQ ID NO: 152 - protein, 9 aa, synthetic construct
  REGION 1..9: note = Dicot plant HPPD modified consensus sequence
  SITE 1: note = MISC_FEATURE - X = A or S
  SITE 2: note = MISC_FEATURE - X = T or V
  SITE 4: note = MISC_FEATURE - X = R, H or K
XXVXLYGDV

SEQ ID NO: 153-155 - skipped (3 entries)

SEQ ID NO: 156 - protein, 8 aa, synthetic construct
  REGION 1..8: note = Dicot plant HPPD modified consensus sequence
  SITE 4: note = MISC_FEATURE - X= N or D
EELXILVD

SEQ ID NO: 157 - protein, 8 aa, synthetic construct
  REGION 1..8: note = Dicot plant HPPD modified consensus sequence
DVVLRFVS

SEQ ID NO: 158 - protein, 7 aa, synthetic construct
  REGION 1..7: note = Dicot plant HPPD modified consensus sequence
GFGAGNF

SEQ ID NO: 159 - protein, 12 aa, synthetic construct
  REGION 1..12: note = Monocot plant HPPD modified consensus sequence
  SITE 1: note = MISC_FEATURE - X = E or A
  SITE 2: note = MISC_FEATURE - X = D or E
  SITE 6: note = MISC_FEATURE - X = A or V
  SITE 10: note = MISC_FEATURE - X = G or A
XXAYRXSVAXGA

SEQ ID NO: 160 - protein, 12 aa, synthetic construct
  REGION 1..12: note = Monocot plant HPPD modified consensus sequence
  SITE 1: note = MISC_FEATURE - X = E or A
  SITE 2: note = MISC_FEATURE - X = D or E
  SITE 6: note = MISC_FEATURE - X = A or V
  SITE 10: note = MISC_FEATURE - X = G or A
XXAFNXSVAXGA

SEQ ID NO: 161 - protein, 12 aa, synthetic construct
  REGION 1..12: note = Monocot plant HPPD modified consensus sequence
  SITE 1: note = MISC_FEATURE - X = E or A
  SITE 2: note = MISC_FEATURE - X = D or E
  SITE 10: note = MISC_FEATURE - X = G or A
XXAFRKSVAXGA

SEQ ID NO: 162 - protein, 12 aa, synthetic construct
  REGION 1..12: note = Monocot plant HPPD modified consensus sequence
  SITE 1: note = MISC_FEATURE - X = E or A
  SITE 2: note = MISC_FEATURE - X = D or E
  SITE 6: note = MISC_FEATURE - X = A or V
  SITE 10: note = MISC_FEATURE - X = G or A
XXAFRXSVAXGA

SEQ ID NO: 163 - protein, 12 aa, synthetic construct
  REGION 1..12: note = Monocot plant HPPD modified consensus sequence
  SITE 1: note = MISC_FEATURE - X = A or E
  SITE 2: note = MISC_FEATURE - X = D or E
  SITE 6: note = MISC_FEATURE - X = A or V
XXAFRXSVAEGA

SEQ ID NO: 164 - protein, 11 aa, synthetic construct
  REGION 1..11: note = Monocot plant HPPD modified consensus sequence
  SITE 10: note = MISC_FEATURE - X= Q or A
VAEGARPAFXP

SEQ ID NO: 165 - protein, 11 aa, synthetic construct
  REGION 1..11: note = Monocot plant HPPD modified consensus sequence
  SITE 3: note = MISC_FEATURE - X= A or G
  SITE 10: note = MISC_FEATURE - X= Q or A
VAXGAIPAFXP

SEQ ID NO: 166 - protein, 11 aa, synthetic construct
  REGION 1..11: note = Monocot plant HPPD modified consensus sequence
  SITE 3: note = MISC_FEATURE - X = A or G
  SITE 10: note = MISC_FEATURE - X = Q or A
VAXGARAAFXP

SEQ ID NO: 167 - protein, 11 aa, synthetic construct
  REGION 1..11: note = Monocot plant HPPD modified consensus sequence
  SITE 3: note = MISC_FEATURE - X = A or G
  SITE 10: note = MISC_FEATURE - X = Q or A
VAXGARPAQXP

SEQ ID NO: 168 - protein, 11 aa, synthetic construct
  REGION 1..11: note = Monocot plant HPPD modified consensus sequence
  SITE 3: note = MISC_FEATURE - X = A or G
VAXGARPAFGP

SEQ ID NO: 169 - protein, 9 aa, synthetic construct
  REGION 1..9: note = Monocot plant HPPD modified consensus sequence
  SITE 5: note = MISC_FEATURE - X= W or R
  SITE 9: note = MISC_FEATURE - X= A or T
FHEFXEFTX

SEQ ID NO: 170 - protein, 9 aa, synthetic construct
  REGION 1..9: note = Monocot plant HPPD modified consensus sequence
  SITE 9: note = MISC_FEATURE - X = A or T
FHEFAEFDX

SEQ ID NO: 171 - protein, 9 aa, synthetic construct
  REGION 1..9: note = Monocot plant HPPD modified consensus sequence
FHEFAEFTD

SEQ ID NO: 172 - protein, 10 aa, synthetic construct
  REGION 1..10: note = Monocot plant HPPD modified consensus sequence
  SITE 2: note = MISC_FEATURE - X= A or T
  SITE 8: note = MISC_FEATURE - X= A or T
DXEDVGTXES

SEQ ID NO: 173 - protein, 10 aa, synthetic construct
  REGION 1..10: note = Monocot plant HPPD modified consensus sequence
  SITE 8: note = MISC_FEATURE - X = A or T
TDEDVGTXES

SEQ ID NO: 174 - protein, 10 aa, synthetic construct
  REGION 1..10: note = Monocot plant HPPD modified consensus sequence
  SITE 2: note = MISC_FEATURE - X = A or T
  SITE 8: note = MISC_FEATURE - X = A or T
  SITE 9: note = MISC_FEATURE - X = F or Y
TXEDVGTXXS

SEQ ID NO: 175 - protein, 13 aa, synthetic construct
  REGION 1..13: note = Monocot plant HPPD modified consensus sequence
  SITE 8: note = MISC_FEATURE - X = P, T or Q
  SITE 9: note = MISC_FEATURE - X = S, P or A
  SITE 10: note = MISC_FEATURE - X = D, N or K
FEFIAPPXXXYYW

SEQ ID NO: 176 - protein, 13 aa, synthetic construct
  REGION 1..13: note = Monocot plant HPPD modified consensus sequence
  SITE 4: note = MISC_FEATURE - X = L or M
  SITE 8: note = MISC_FEATURE - X = P, T or Q
  SITE 9: note = MISC_FEATURE - X = S, P or A
  SITE 10: note = MISC_FEATURE - X = D, N or K
FEFXNPPXXXYYW

SEQ ID NO: 177 - protein, 13 aa, synthetic construct
  REGION 1..13: note = Monocot plant HPPD modified consensus sequence
  SITE 4: note = MISC_FEATURE - X = L or M
  SITE 8: note = MISC_FEATURE - X = P ,T or Q
  SITE 9: note = MISC_FEATURE - X = S, P or A
  SITE 10: note = MISC_FEATURE - X = D, N or K
FEFXAVPXXXYYW

SEQ ID NO: 178 - protein, 13 aa, synthetic construct
  REGION 1..13: note = Monocot plant HPPD modified consensus sequence
  SITE 4: note = MISC_FEATURE - X = L or M
  SITE 9: note = MISC_FEATURE - X = S, P or A
  SITE 10: note = MISC_FEATURE - X = D, N or K
FEFXAPPGXXYYW

SEQ ID NO: 179 - protein, 13 aa, synthetic construct
  REGION 1..13: note = Monocot plant HPPD modified consensus sequence
  SITE 4: note = MISC_FEATURE - X = L or M
  SITE 8: note = MISC_FEATURE - X = P, T or Q
  SITE 10: note = MISC_FEATURE - X = D, N or K
FEFXAPPXGXYYW

SEQ ID NO: 180 - protein, 11 aa, synthetic construct
  REGION 1..11: note = Monocot plant HPPD modified consensus sequence
  SITE 4: note = MISC_FEATURE - X = L or M
  SITE 8: note = MISC_FEATURE - X = P, T or Q
  SITE 9: note = MISC_FEATURE - X = D, N or K
FEFXAPPXYYW

SEQ ID NO: 181 - protein, 10 aa, synthetic construct
  REGION 1..10: note = Monocot plant HPPD modified consensus sequence
  SITE 1: note = MISC_FEATURE - X = D, N or K
XYYDGVRRGV

SEQ ID NO: 182 - protein, 10 aa, synthetic construct
  REGION 1..10: note = Monocot plant HPPD modified consensus sequence
  SITE 1: note = MISC_FEATURE - X = D, N or K
  SITE 4: note = MISC_FEATURE - X = D or E
XYYXGVRLGV

SEQ ID NO: 183 - protein, 8 aa, synthetic construct
  REGION 1..8: note = Monocot plant HPPD modified consensus sequence
  SITE 4: note = MISC_FEATURE - X = N or D
QELXVLVD

SEQ ID NO: 184 - protein, 8 aa, synthetic construct
  REGION 1..8: note = Monocot plant HPPD modified consensus sequence
  SITE 3: note = MISC_FEATURE - X = Y or F
VDXDDQGV

SEQ ID NO: 185 - protein, 8 aa, synthetic construct
  REGION 1..8: note = Monocot plant HPPD modified consensus sequence
  SITE 6: note = MISC_FEATURE - X = A or G
VDRDDXGV

SEQ ID NO: 186 - protein, 9 aa, synthetic construct
  REGION 1..9: note = Monocot plant HPPD modified consensus sequence
EMIQRNGCM

SEQ ID NO: 187 - protein, 9 aa, synthetic construct
  REGION 1..9: note = Monocot plant HPPD modified consensus sequence
EMIQRINCM

SEQ ID NO: 188 - protein, 9 aa, synthetic construct
  REGION 1..9: note = Monocot plant HPPD modified consensus sequence
EMIQRIGFM

SEQ ID NO: 189 - protein, 9 aa, synthetic construct
  REGION 1..9: note = Monocot plant HPPD modified consensus sequence
EMIQRIGCG

SEQ ID NO: 190 - protein, 8 aa, synthetic construct
  REGION 1..8: note = Monocot plant HPPD modified consensus sequence
EMIQRIGC

SEQ ID NO: 191 - protein, 9 aa, synthetic construct
  REGION 1..9: note = Monocot plant HPPD modified consensus sequence
  SITE 7: note = MISC_FEATURE - X = K, S or V
FMEKDEXGQ

SEQ ID NO: 192 - protein, 9 aa, synthetic construct
  REGION 1..9: note = Monocot plant HPPD modified consensus sequence
  SITE 2: note = MISC_FEATURE - X = T or V
AXVELYGDV

SEQ ID NO: 193-195 - skipped (3 entries)

SEQ ID NO: 196 - protein, 10 aa, synthetic construct
  REGION 1..10: note = Monocot plant HPPD modified consensus sequence
  SITE 7: note = MISC_FEATURE - X = E or S
YQKGACXGFG

SEQ ID NO: 197 - protein, 9 aa, synthetic construct
  REGION 1..9: note = Monocot plant HPPD modified consensus sequence
DVVLRFVSY

SEQ ID NO: 198 - protein, 7 aa, synthetic construct
  REGION 1..7: note = Monocot plant HPPD modified consensus sequence
GFGAGNF

SEQ ID NO: 199 - protein, 8 aa, synthetic construct
  REGION 1..8: note = Dicot plant HPPD consensus sequence
EELGILVD

SEQ ID NO: 200 - protein, 8 aa, synthetic construct
  REGION 1..8: note = Dicot plant HPPD consensus sequence
DVVLRYVS

SEQ ID NO: 201 - protein, 7 aa, synthetic construct
  REGION 1..7: note = Dicot plant HPPD consensus sequence
GFGKGNF

SEQ ID NO: 202 - protein, 10 aa, synthetic construct
  REGION 1..10: note = Monocot plant HPPD consensus sequence
YQKGACGGFG

SEQ ID NO: 203 - protein, 9 aa, synthetic construct
  REGION 1..9: note = Monocot plant HPPD consensus sequence
DVVLRYVSY

SEQ ID NO: 204 - protein, 7 aa, synthetic construct
  REGION 1..7: note = Monocot plant HPPD consensus sequence
GFGKGNF

SEQ ID NO: 205 - protein, 445 aa, Arabidopsis thaliana
MGHQNAAVSENQNHDDGAASSPGFKLVGFSKFVRKNPKSDKFKVKRFHHIEFWCGDATNVARRFSWGLGMRFSAKSDLSTGNMVHASYLLTSGDLRFLFTAPYSPSLSAGEIKPTTTASIPSFDHGSCRSFFSSHGLGVRAVAIEVEDAESAFSISVANGAIPSSPPIVLNEAVTIAEVKLYGDVVLRYVSYKAEDTEKSEFLPGFERVEDASSFPLDYGIRRLDHAVGNVPELGPALTYVAGFTGFHQFAEFTADDVGTAESGLNSAVLASNDEMVLLPINEPVHGTKRKSQIQTYLEHNEGAGLQHLALMSEDIFRTLREMRKRSSIGGFDFMPSPPPTYYQNLKKRVGDVLSDDQIKECEELGILVDRDDQGTLLQIFTKPLGDRPTIFIEIIQRVGCMMKDEEGKAYQSGGCGGFGKGNFSELFKSIEEYEKTLEAKQLVG

SEQ ID NO: 206 - protein, 447 aa, Glycine max
MPIPMCNEIQAQAQAQAQPGFKLVGFKNFVRTNPKSDRFQVNRFHHIEFWCTDATNASRRFSWGLGMPIVAKSDLSTGNQIHASYLLRSGDLSFLFSAPYSPSLSAGSSAASSASIPSFDAATCLAFAAKHGFGVRAIALEVADAEAAFSASVAKGAEPASPPVLVDDRTGFAEVRLYGDVVLRYVSYKDAAPQAPHADPSRWFLPGFEAAASSSSFPELDYGIRRLDHAVGNVPELAPAVRYLKGFSGFHEFAEFTAEDVGTSESGLNSVVLANNSETVLLPLNEPVYGTKRKSQIETYLEHNEGAGVQHLALVTHDIFTTLREMRKRSFLGGFEFMPSPPPTYYANLHNRAADVLTVDQIKQCEELGILVDRDDQGTLLQIFTKPVGDRPTIFIEIIQRIGCMVEDEEGKVYQKGACGGFGKGNFSELFKSIEEYEKTLEAKRTA

SEQ ID NO: 207 - protein, 447 aa, Solanum tuberosum
MGKQAATATATATATADDQQLPADIEDKYKLVGFNNFVRTNPRSDLFNVKRFHHIEFWCGDATNTARRFSWGLGLPIAAKSDLSTGNSVHASYLLRSSSSQLQFLFTAPYSPAISTPSSSSIPTFSVSSHRSFTETHGLGVRAIALEVENSRLAFSTCVSHGAKPVSEPVILNDEVVISEVHLYGDVVLRFVSFLKDSNRFVFLPGFELVEGAQLDYGIRRLDHAVGNVPQLGPVVEYIKSFTGFHEFAEFTAEDVGTAESGLNSVVLANNDETVLFPLNEPVYGTKRKSQIQTYLEHNEGAGVQHLALVTEDIFRTLREMRKRSGIGGFEFMPSPPPTYYKNLKSRAGDILSDEQIKECEELGILVDRDDQGTLLQIFTKPVGDRPTIFLEIIQRIGCMLQNAEGELYQKGGCGGFGKGNFSELFKSIEEYEKTLEAKQNTQVAIA

SEQ ID NO: 208 - protein, 440 aa, Brassica napus
MGHENAAVSESQHHDDAASAASPGFKLVGFSKFVRKNPKSDKFKVKRFHHIEFWCGDATNVARRFSWGLGMRFSAKSDLSTGNMVHASYLLTSGDLRFLFTAPYSPSLSAGETSTASIPSFDHVSCRSFFSSHGLGVRAVAIEVEDAESAFSISVANGAVPSSPPNVLNGAVTIAEVKLYGDVVLRYVSYHNGAVNFLPGFESVDDTSSFPLDYGIRRLDHAVGNVPELGPALTYLAGFTGFHQFAEFTADDVGTAESGLNSAVLANNDEMVLLPVNEPVHGTKRKSQIQTFLEHNEGAGLQHLALMSEDIFRTLREMRKRSGVGGFDFMPSPPPTYYKNLKKRIGDVLSDEQIRECEELGILVDRDDQGTLLQIFTKPLGDRPTIFIEIIQRVGCMKKDEEGKVYQSGGCGGFGKGNFSELFKSIEEYEKTLEAKQLVG

SEQ ID NO: 209 - protein, 435 aa, Medicago sativa
MAIETETQTQTGFKLVGFKNFVRANPKSDRFKVKRFHHVEFWCTDATNTARRFSHGLGMPIVAKSDLSTGNQTHASYLLRSGDLNFLFSAAYSPSISLSSPSSTAAIPTFSASNCFSFSASHGLAVRAVAVEVEDAELAFTTSVNNGAIPSSPPVILENHVKLAEVRLYGDVVLRYVSYNDPNPNQNPNLLFLPGFERVSDESSNSSLDFGIRRLDHAVGNVPELSSAVKYVKDFTGFHEFAEFTAEDVGTSESGLNSVVLANNEETVLLPMNEPVYGTKRKSQIETYLEHNEGAGLQHLALKSEDIFRTLREMRKRSGVGGFEFMPSPPVTYYRNLKNRVGDVLSDEQIKECEELGILVDRDDQGTLLQIFTKPIGDRPTIFIEIIQRVGCMLKDEEGKEYQKGGCGGFGKGNFSELFKSIEEYEKTLETRRTA

SEQ ID NO: 210 - protein, 436 aa, Gossypium hirsutum
MVQTNQSGSGNDFKLVGFSNFVRSNPKSDRFTVKRFHHIEFWCTDATNVARRFSWGLGMQFVAKSDLSTGNLTHASYLLRSRDLNFLFTAPYSPSIAVAQNLSPQSTASIPSFDHSLCRSFAATHGLGVRAIAIEVDDAETAFTTSVTHGALPFCPPTPLGDVATIAEVKLYGDVVLRYVSYTTTINSDHDFLPGFEKIEDTLSYPLDYGLRRLDHAVGNVPELGPAVSYVKSFTGFHEFAEFTAEDVGTSESGLNSVVLANNEEMVLLPMNEPVFGTKRKSQIQTYLEHNEGAGVQHLALVSEDIFKTLREMRKRSFVGGFEFMPSPPPTYYKKLKQRAGDILSDEQIKECEELGILVDRDDQGTLLQIFTKPVGDRPTIFIEIIQRIGCMVKDEEGKQYQKGGCGGFGKGNFSELFKSIEEYEKSLEAKQSQNP

SEQ ID NO: 211 - protein, 434 aa, Beta vulgaris
MGTLSPQPQTSPSQFKLVGYSNFIRVNPLSDLFPVKRFHHVEFWCGDATNVSRRFSWGLGMPIVAKSDLSTGNSVHASYLLRSGELHFLFTSPYSPSLSSPSSATIPTFSFSLFTSFLTSHGLAVRAVAVEVQDAEAAFHISVSNGASPVSPPIRLHDGVVLSEVHLYGDVVLRYVSTSSVVDGIFLPGFEEMLGESSFQELDFGLRRLDHAVGNVPELAPAIEYVKKFTGFHEFAEFTTEDVGTSESGLNSAVVANNDETVLFPMNEPVYGTKRKSQIQTYLEHNEGAGVQHLALMSEDIFWTLREMRKRSGLGGFEFMPSPPPTYYRNLRNRAADVLSEEQMKECEELGILVDKDDQGTLLQIFTKPIGDRPTMFIEIIQRIGCMMKDEEGKVYQKGGCGGFGKGNFSELFKSIEEYEKTLERKPIADTNAT

SEQ ID NO: 212 - protein, 418 aa, Zea mays
ASAAEQAAFRLVGHRNFVRFNPRSDRFHTLAFHHVELWCADAASAAGRFSFGLGAPLAARSDLSTGNSAHASLLLRSGSLSFLFTAPYAHGADAATAALPSFSAAAARRFAADHGLAVRAVALRVADAEDAFRASVAAGARPAFGPVDLGRGFRLAEVELYGDVVLRYVSYPDGAAGEPFLPGFEGVASPGAADYGLSRFDHIVGNVPELAPAAAYFAGFTGFHEFAEFTTEDVGTAESGLNSMVLANNSENVLLPLNEPVHGTKRRSQIQTFLDHHGGPGVQHMALASDDVLRTLREMQARSAMGGFEFMAPPTSDYYDGVRRRAGDVLTEAQIKECQELGVLVDRDDQGVLLQIFTKPVGDRPTLFLEIIQRIGCMEKDEKGQEYQKGGCGGFGKGNFSQLFKSIEDYEKSLEAKQ

SEQ ID NO: 213 - protein, 446 aa, Oryza sativa
MPPTPTPTATTGAVSAAAAAGENAGFRLVGHRRFVRANPRSDRFQALAFHHVELWCADAASAAGRFAFALGAPLAARSDLSTGNSAHASLLLRSASVAFLFTAPYGGDHGVGADAATTASIPSFSPGAARRFAADHGLAVHAVALRVADAADAFRASVAAGARPAFQPADLGGGFGLAEVELYGDVVLRFVSHPDGADAPFLPGFEGVSNPGAVDYGLRRFDHVVGNVPELAPVAAYISGFTGFHEFAEFTAEDVGTAESGLNSVVLANNAETVLLPLNEPVHGTKRRSQIQTYLDHHGGPGVQHIALASDDVLGTLREMRARSAMGGFEFLAPPPPNYYDGVRRRAGDVLSEEQINECQELGVLVDRDDQGVLLQIFTKPVGDRPTFFLEMIQRIGCMEKDESGQEYQKGGCGGFGKGNFSELFKSIEEYEKSLEAKQAPTVQGS

SEQ ID NO: 214 - protein, 440 aa, synthetic construct
  REGION 1..440: note = Artificial sequence
MPPTPATATGAAAAAVTPEHAARSFPRVVRVNPRSDRFPVLSFHHVELWCADAASAAGRFSFALGAPLAARSDLSTGNSAHASLLLRSGALAFLFTAPYAPPPQEAATAAATASIPSFSADAARTFAAAHGLAVRSVGVRVADAAEAFRVSVAGGARPAFAPADLGHGFGLAEVELYGDVVLRFVSYPDETDLPFLPGFERVSSPGAVDYGLTRFDHVVGNVPEMAPVIDYMKGFLGFHEFAEFTAEDVGTTESGLNSVVLANNSEAVLLPLNEPVHGTKRRSQIQTYLEYHGGPGVQHIALASNDVLRTLREMRARTPMGGFEFMAPPQAKYYEGVRRIAGDVLSEEQIKECQELGVLVDRDDQGVLLQIFTKPVGDRPTFFLEMIQRIGCMEKDEVGQEYQKGGCGGFGKGNFSELFKSIEDYEKSLEVKQSVVAQKS

(19) United States
(12) Patent Application Publication
(10) Pub. No.: US 2024/0247278 A1
(43) Pub. Date: Jul. 25, 2024

(54) COMPOSITIONS AND METHODS FOR CONTROLLING PLANT GROWTH

(71) Applicant: PLANTARC Bio LTD., Raanana (IL)

(72) Inventors: Dror Shalitin, Raanana (IL); Noam Grimberg, Kibutz Lehavot Haviva (IL)

(73) Assignee: PLANTARC Bio LTD., Raanana (IL)

(21) Appl. No.: 18/433,835

(22) Filed: Feb. 6, 2024

Related U.S. Application Data
(63) Continuation of application No. 17/683,747, filed on Mar. 1, 2022, now Pat. No. 11,926,837.
Application No. 17/683,747 is a continuation of application No. PCT/IL2020/050967, filed on Sep. 3, 2020.
(60) Provisional application No. 62/896,312, filed on Sep. 5, 2019.

(51) Int. Cl. C12N 15/82

(52) U.S. Cl. CPC C12N 15/8274; C12Y 113/11027

(57) ABSTRACT

A plant containing a nucleic acid sequence encoding a protein conferring resistance to a herbicide including one or more HPPD inhibitors, wherein the nucleic acid sequence encodes for a modified and/or exogenous HPPD enzyme.

### SEQUENCE LISTING

A sequence listing in electronic (XML file) format is filed with this application and incorporated herein by reference. The name of the XML file is “2023_1958_sequence_listing.xml”; the file was created on Feb. 6, 2024; the size of the file is 335,360 bytes.

### FIELD OF INVENTION

The present invention relates to plants, plant cells, tissues, and seeds that have been genetically modified to express a fungus derived HPPD or variant thereof and/or to plants in which the endogenous HPPD has been edited (e.g. by CRISPR/Cas technology, TALLEN, Zink-finger etc.) to include fungus derived motifs and/or mutations, thereby providing plants with an HPPD that confers resistance or tolerance to HPPD inhibitors.

### BACKGROUND OF THE INVENTION

Weeds have been the major biotic cause of crop yield loses since the origin of agriculture. Weeds compete with crops for space, nutrients, water and light, and the potential of weed damages is estimated as 34% loss of crop yield, on average, world-wide [Oerke, E-C., 2006].

Herbicides are the most commonly used and effective weed control tools. Due to the intense selection pressure exerted by herbicides, herbicide resistance is constantly growing and, as of 2016 there are over 470 weed biotypes currently identified as being herbicide resistant to one or more herbicides by The International Survey of Herbicide Resistant Weeds (weedscience.org). Weeds compete with productive crops or pasture, ultimately converting productive land into unusable scrub. Moreover, weeds can be poisonous, distasteful, produce burrs, thorns or otherwise interfere with the use and management of desirable plants by contaminating harvests or interfering with livestock.

4-Hydroxyphenylpyruvate dioxygenase (HPPD) inhibitors (HPPD inhibitors) are a class of herbicides that inhibit plant growth by blocking HPPD, an enzyme catalyzes the conversion of p-hydroxyphenylpyruvate (HPP) into homogentisate (HGA), a key precursor of a-tocopherol and plastoquinone in plant's tyrosine degradation pathway. Preventing the breakdown of tyrosine causes three major impacts on the treated plant: excess of tyrosine which stunts growth; oxidative damage due to lack of tocopherols (vitamin E); and chlorophyll destruction due to lack of carotenoids. Plants turn white due to a complete loss of chlorophyll, which has led compounds of this class to be classified as a “bleaching herbicide”.

Herbicides that act by inhibiting HPPD are well known, and include isoxazoles, diketonitriles, triketones, and pyrazolinates (Hawkes “Hydroxyphenylpyruvate Dioxygenase (HPPD)—The Herbicide Target.” In Modern Crop Protection Compounds. Eds. Kramer and Schirmer. Weinheim, Germany: Wiley-VCH, 2007. Ch. 4.2, pp. 211-220).

HPPD inhibitors were first brought to market in 1980, although their mechanism of action was not understood until the late 1990s. They were originally used primarily in Japan in rice production, but since the late 1990s have been used in Europe and North America for corn, soybeans, and cereals, and since the 2000s have become more important as weeds have become resistant to glyphosate and other herbicides.

Specifically, tembotrione, mesotrione and isoxaflutole provide powerful residual control of more than 65 grass and broadleaf weeds with unsurpassed crop safety in all types of corn. It is also effective on the toughest broadleaf weeds, including glyphosate-, PPO-, ALS- and dicamba-resistant weeds.

HPPD inhibitors, can only be used in crops if the crop is resistant/tolerant to the herbicide. The treatment of plants susceptible to HPPD inhibition, such as, but not limited to, broad-leaf plants, is thus limited. It has been of particular difficulty to achieve a resistance that provides commercial levels of tolerance to at least some desirable HPPD-inhibitor herbicides. Accordingly, new methods and compositions for conferring HPPD herbicide tolerance upon various crops and crop varieties are needed.

### SUMMARY OF THE INVENTION

The present invention provides plants, plant cells, tissues, and seeds that have been genetically modified with a fungus derived HPPD or variant thereof and/or to plants in which the endogenous HPPD has been edited (e.g. by CRISPR/Cas technology, TALLEN, Zink-finger etc.) to include fungus derived motifs and/or mutations, thereby providing plants with an HPPD that confers resistance or tolerance to herbicides.

Compositions and methods for conferring hydroxyphenyl pyruvate dioxygenase (HPPD) herbicide resistance or tolerance to plants are provided. The compositions include nucleotide and amino acid sequences for HPPD polypeptides. In certain embodiments, the polypeptides of the invention are fungal HPPDs or HPPD derivatives that when expressed in plants confer their resistance or tolerance to herbicides that inhibit HPPD.

According to some embodiments, the HPPD, expressed in the plant, comprises one or more of the amino acid sequences set forth in SEQ ID NO: 9 (EAVYNKAVAEGA), SEQ ID NO: 10 (VAEGAIAVQGP), SEQ ID NO: 11(FHRFWSVDD), SEQ ID NO: 12 (DDSQICTEFS), SEQ ID NO: 13 (VEFINVPTTYY), SEQ ID NO: 14 (TYYDTMRQRLKT), SEQ ID NO: 15 (QRLNILID), SEQ ID NO: 16 (IDYDEAGY), SEQ ID NO: 17 (EIIQRNNF), SEQ ID NO: 18 (NNFEGFG), SEQ ID NO: 19 (AVICTYGDT), SEQ ID NO: 20 (DTTHTLINR), SEQ ID NO: 21 (EMVSACA), SEQ ID NO: 22 (CAFYEQC), SEQ ID NO: 23 (GFGAGNF), SEQ ID NO: 24 (TPDNFA); SEQ ID NO: 25 (DDVFAAAVQNGA), SEQ ID NO: 26 (VQNGAVAVSQP), SEQ ID NO: 27 (FHRFRSVDD), SEQ ID NO: 28 (DDKDICTDYS), SEQ ID NO: 29 (VEFIKVPPTYY), SEQ ID NO: 30 (TYYDNMWMRLKK), SEQ ID NO: 31 (KKLDILID), SEQ ID NO: 32 (IDFDEGGY), SEQ ID NO: 33 (NNFSGFG), SEQ ID NO: 34 (ATIRTYGDT), SEQ ID NO: 35 (DTTHTLIQR), SEQ ID NO: 36 (CAYYEKV), SEQ ID NO: 37 (QSDNLP) or any combination thereof. Each possibility is a separate embodiment.

According to some embodiments, the HPPD, expressed in the plant, comprises one or more of the amino acid sequences set forth in SEQ ID NO: 9 (EAVYNKAVAEGA), SEQ ID NO: 10 (VAEGAIAVQGP), SEQ ID NO: 11 (FHRFWSVDD), SEQ ID NO: 12 (DDSQICTEFS), SEQ ID NO: 13 (VEFINVPTTYY), SEQ ID NO: 14 (TYYDTMRQRLKT), SEQ ID NO: 15 (QRLNILID), SEQ ID NO: 16 (IDYDEAGY), SEQ ID NO: 17 (EIIQRNNF), SEQ ID NO: 18 (NNFEGFG), SEQ ID NO: 19 (AVICTYGDT), SEQ ID NO: 20 (DTTHTLINR), SEQ ID NO: 21 (EMVSACA), SEQ ID NO: 22 (CAFYEQC), SEQ ID NO: 23 (GFGAGNF), SEQ ID NO: 24 (TPDNFA) or any combination thereof. Each possibility is a separate embodiment.

According to some embodiments, the HPPD, expressed in the plant, comprises one or more of the amino acid sequences set forth in SEQ ID NO: 25 (DDVFAAAVQNGA), SEQ ID NO: 26 (VQNGAVAVSQP), SEQ ID NO: 27 (FHRFRSVDD), SEQ ID NO: 28 (DDKDICTDYS), SEQ ID NO: 29 (VEFIKVPPTYY), SEQ ID NO: 30 (TYYDNMWMRLKK), SEQ ID NO: 31 (KKLDILID), SEQ ID NO: 32 (IDFDEGGY), SEQ ID NO: 33 (NNFSGFG), SEQ ID NO: 34 (ATIRTYGDT), SEQ ID NO: 35 (DTTHTLIQR), SEQ ID NO: 36 (CAYYEKV), SEQ ID NO: 37 (QSDNLP) or any combination thereof. Each possibility is a separate embodiment.

According to some embodiments, the HPPD, expressed in the plant, comprises all of the amino acid sequences set forth in SEQ ID NOs: 9-24 and/or all of the amino acid sequences set forth in SEQ ID NOs: 25-37.

According to some embodiments, the amino acid sequences set forth in SEQ ID NOs: 9-37 constitute motifs. As used herein, the term “motif” refers to an amino acid sequence which can form secondary structure elements that either have a particular functional significance or define a portion of an independently folded domain, such as, for example, a loop structure. According to some embodiments, the HPPD, expressed in the plant, comprises one or more motifs or is genetically modified to express one or more motifs or is genetically edited to express one or more motifs. According to some embodiments, the one or more motifs comprises or consists of an amino acid sequence set forth in any one or more of SEQ ID NOs: 9-37 or 9-24. Each possibility is a separate embodiment. According to some embodiments, the HPPD SEQ ID NOs: 9-24 form 9 motifs.

According to some embodiments, motif 1-9 are derived from SEQ ID NO: 1.

According to some embodiments, motif 1 includes SEQ ID NO: 9 and 10 or modified versions thereof. According to some embodiments, motif 1 has the amino acid sequence set forth in SEQ ID NO: 38 (EAVYNKAVAEGAIAVQGP).

According to some embodiments, motif 2 includes SEQ ID NO: 11 and 12 or modified versions thereof. According to some embodiments, motif 2 has the amino acid sequence set forth in SEQ ID NO: 39 (FHRFWSVDDSQICTEFS).

According to some embodiments, motif 3 includes SEQ ID NO: 13 and 14 or modified versions thereof. According to some embodiments, motif 3 has the amino acid sequence set forth in SEQ ID NO: 40 (VEFINVPTTYYDTMRQRLKT).

According to some embodiments, motif 4 includes SEQ ID NO: 15 and 16 or modified versions thereof. According to some embodiments, motif 4 has the amino acid sequence set forth in SEQ ID NO: 41 (QRLNILIDYDEAGY).

According to some embodiments, motif 5 includes SEQ ID NO: 17 and 18 or modified versions thereof. According to some embodiments, motif 5 has the amino acid sequence set forth in SEQ ID NO: 42 (EIIQRNNFEGFG).

According to some embodiments, motif 6 includes SEQ ID NO: 19 and 20 or modified versions thereof. According to some embodiments, motif 6 has the amino acid sequence set forth in SEQ ID NO: 43 (AVICTYGDTTHTLINR).

According to some embodiments, motif 7 includes SEQ ID NO: 21 and 22 or modified versions thereof. According to some embodiments, motif 7 has the amino acid sequence set forth in SEQ ID NO: 44 (EMVSACAFYEQC).

According to some embodiments, motif 8 includes SEQ ID NO: 23 or modified versions thereof. According to some embodiments, motif 8 has the amino acid sequence set forth in SEQ ID NO: 45 (GFGAGNF).

According to some embodiments, motif 9 includes SEQ ID NO: 24 or modified versions thereof. According to some embodiments, motif 9 has the amino acid sequence set forth in SEQ ID NO: 46 (TPDNFA).

According to some embodiments, the HPPD, expressed in the plant, comprises at least one, at least two, at least three, at least four or more of the amino acid sequences set forth in SEQ ID NOs: 38-46. According to some embodiments, the HPPD, expressed in the plant, comprises all of the amino acid sequences set forth in SEQ ID NOs: 38-46.

According to some embodiments, motif 1-9 are derived from SEQ ID NO: 3.

According to some embodiments, motif 1 includes SEQ ID NO: 25 and 26 or modified versions thereof. According to some embodiments, motif 1 has the amino acid sequence set forth in SEQ ID NO: 47 (DDVFAAAVQNGAVAVSQP).

According to some embodiments, motif 2 includes SEQ ID NO: 27 and 28 or modified versions thereof. According to some embodiments, motif 2 has the amino acid sequence set forth in SEQ ID NO: 48 (FHRFRSVDDKDICTDYS).

According to some embodiments, motif 3 includes SEQ ID NO: 29 and 30 or modified versions thereof. According to some embodiments, motif 3 has the amino acid sequence set forth in SEQ ID NO: 49 (VEFIKVPPTYYDNMWMRLKK).

According to some embodiments, motif 4 includes SEQ ID NO: 31 and 32 or modified versions thereof. According to some embodiments, motif 4 has the amino acid sequence set forth in SEQ ID NO: 50 (KKLDILIDFDEGGY).

According to some embodiments, motif 5 includes SEQ ID NO: 33 or modified versions thereof. According to some embodiments, motif 5 has the amino acid sequence set forth in SEQ ID NO: 51 (EIIQRNNFSGFG).

According to some embodiments, motif 6 includes SEQ ID NO: 34 and 35 or modified versions thereof. According to some embodiments, motif 6 has the amino acid sequence set forth in SEQ ID NO: 52 (ATIRTYGDTTHTLIQR).

According to some embodiments, motif 7 includes SEQ ID NO: 36 or modified versions thereof. According to some embodiments, motif 7 has the amino acid sequence set forth in SEQ ID NO: 53 (EMEKVCAYYEKV).

According to some embodiments, motif 9 includes SEQ ID NO: 37 or modified versions thereof. According to some embodiments, motif 9 has the amino acid sequence set forth in SEQ ID NO: 54 (QSDNLP).

According to some embodiments, the HPPD, expressed in the plant, comprises at least a fungal derived motif 1. According to some embodiments, the fungal derived motif 1 may include any one of the SEQ ID NOs set forth in SEQ ID NOs: 9, 10, 25, 26, 38, 47, 119-128 or 159-168. Each possibility is a separate embodiment.

According to some embodiments, the HPPD, expressed in the plant, comprises at least a fungal derived motif 2. According to some embodiments, the fungal derived motif 2 may include any one of the SEQ ID Nos set forth in SEQ ID NOs: 11, 12, 27, 28, 39, 48, 129-134 or 169-174. Each possibility is a separate embodiment.

According to some embodiments, the HPPD, expressed in the plant, comprises at least a fungal derived motif 3. According to some embodiments, the fungal derived motif 3 may include any one of the SEQ ID Nos set forth in SEQ ID NOs: 13, 14, 29, 30, 40, 49, 135-142 or 175-182. Each possibility is a separate embodiment.

According to some embodiments, the HPPD, expressed in the plant, comprises at least a fungal derived motif 4. According to some embodiments, the fungal derived motif 4 may include any one of the SEQ ID Nos set forth in SEQ ID NOs: 15, 16, 31, 32, 41, 50, 143, 144, 156 or 183-185. Each possibility is a separate embodiment.

According to some embodiments, the HPPD, expressed in the plant, comprises at least a fungal derived motif 5. According to some embodiments, the fungal derived motif 5 may include any one of the SEQ ID Nos set forth in SEQ ID NOs: 17, 18, 33, 42, 51, 145-151, 186-191 or 196. Each possibility is a separate embodiment.

According to some embodiments, the HPPD, expressed in the plant, comprises at least a fungal derived motif 6. According to some embodiments, the fungal derived motif 6 may include any one of the SEQ ID Nos set forth in SEQ ID NOs: 19, 20, 34, 35, 43, 52, 152, 157, 192 or 197. Each possibility is a separate embodiment.

According to some embodiments, the HPPD, expressed in the plant, comprises at least a fungal derived motif 7. According to some embodiments, the fungal derived motif 7 may include any one of the SEQ ID Nos set forth in SEQ ID Nos 21, 22, 36, 44, 53, 153, 154, 193 or 194. Each possibility is a separate embodiment.

According to some embodiments, the HPPD, expressed in the plant, comprises at least a fungal derived motif 8. According to some embodiments, the fungal derived motif 8 may include any one of the SEQ ID Nos set forth in SEQ ID Nos 23, 45, 158 or 198. Each possibility is a separate embodiment.

According to some embodiments, the HPPD, expressed in the plant, comprises at least a fungal derived motif 9. According to some embodiments, the fungal derived motif 9 may include any one of the SEQ ID Nos set forth in SEQ ID Nos 24, 37, 46, 54, 155 or 195. Each possibility is a separate embodiment.

According to some embodiments, the term “fungal derived motif” refers to a motif coming from a fungus and/or to an endogenous motif at least partially modified to mimic the equivalent motif of the fungus.

According to some embodiments, the plant is a dicot plant and wherein the dicot plant has been genetically modified to express an HPPD enzyme encoded by an amino acid sequence having any of the amino acid sequences set forth in SEQ ID NO: 119-158. Each possibility is a separate embodiment.

According to some embodiments, the plant is a dicot plant and wherein the dicot plant has been genetically modified to express an HPPD enzyme encoded by an amino acid sequence having any of the amino acid sequences set forth in SEQ ID NO: 159-198. Each possibility is a separate embodiment.

According to some embodiments, the HPPD enzyme comprises 9 helices, wherein helix 7 of the modified HPPD enzyme disclosed herein is altered. Without being bound by any theory the altered helix 7 results in a modified active site and thus in lower affinity to HPPD inhibitors.

In particular embodiments, the HPPD, expressed in the plant, comprises an amino acid sequence set forth in SEQ ID NO: 1, 3 or 5-8 or polypeptides having at least about 99, 98, 97, 96, 95, 94, 93, 92, 91 or 90% sequence identity to SEQ ID NO: 1, 3, or 5-8 that exhibit HPPD enzyme activity. According to some embodiments, the nucleotide sequences encoding the HPPD comprises the nucleotide sequences set forth in SEQ ID NO: 2, 4 or parts or homologs thereof encoding the HPPDs having the amino acid sequence set forth in SEQ ID NO: 1, 3, or 5-8.

According to some embodiments, the HPPD, expressed in the plant includes a sequence having at least 80%, at least 85%, at least 90%, at least 95% or at least 98% sequence homology to a sequence set forth in a referred to sequence ID NO. Each possibility is a separate embodiment. As a non-limiting example, the HPPD, expressed in the plant may include an amino acid sequence having at least 80%, at least 85%, at least 90%, at least 95% or at least 98% homology to any of the amino acid sequences set forth in SEQ ID NOs: 9-54.

According to some embodiments, the herein disclosed HPPD, when expressed in plants, confers resistance to a variety of herbicides that inhibit HPPD, such as at least two different HPPDs, at least three HPPDs or at least four HPPDs.

According to some embodiments, the HPPD protein may be a non-naturally occurring modified HPPD protein. According to some embodiments, the HPPD protein may be a synthetic protein. According to some embodiments, the HPPD protein may be an HPPD mutant. According to some embodiments, the HPPD protein may be an HPPD protein modified by gene editing e.g. using CRISPR-CAS technologies.

According to some embodiments, the HPPD protein may be a modified version of the HPPD protein endogenous to G. max (Gm_HPPD) set forth in SEQ ID NO: 118, wherein the modified version has HPPD enzymatic activity and comprises the amino acid sequence set forth in SEQ ID NO: 55 (EAAFSASVAKGA), wherein the first F is replaced with any other amino acid, particularly with Y; and/or wherein the first S is replaced with any other amino acid, particularly with N; and/or wherein the third A is replaced with any other amino acid, particularly with K; and/or wherein the second S is replaced with any other amino acid, particularly with A; and/or wherein the first K is replaced with any other amino acid, particularly with E.

According to some embodiments, the HPPD protein may be a modified version of the HPPD protein endogenous to G. max (Gm_HPPD), wherein the modified version has HPPD enzymatic activity and comprises the amino acid sequence set forth in SEQ ID NO: 56 (VAKGAEPASPP), wherein the first K is replaced with any other amino acid, particularly with E; and/or wherein the first E is replaced with any other amino acid, particularly with I; and/or wherein the first P is replaced with any other amino acid, particularly with A; and/or wherein the first S is replaced with any other amino acid, particularly with Q; and/or wherein the second P is replaced with any other amino acid, particularly with G.

According to some embodiments, the HPPD protein may be a modified version of the HPPD protein endogenous to G. max (Gm_HPPD), wherein the modified version has HPPD enzymatic activity and comprises the amino acid sequence set forth in SEQ ID NO: 57 (FHEFAEFTA), wherein the first A is replaced with any other amino acid, particularly with W or R; and/or wherein the first T is replaced with any other amino acid, particularly with D; and/or wherein the second A is replaced with any other amino acid, particularly with D.

According to some embodiments, the HPPD protein may be a modified version of the HPPD protein endogenous to G. max (Gm_HPPD), wherein the modified version has HPPD enzymatic activity and comprises the amino acid sequence set forth in SEQ ID NO: 58 (TAEDVGTSES), wherein the first T is replaced with any other amino acid, particularly with D; and/or wherein the first A is replaced with any other amino acid, particularly with D; and/or wherein the first E is replaced with any other amino acid, particularly with For Y.

According to some embodiments, the HPPD protein may be a modified version of the HPPD protein endogenous to G. max (Gm_HPPD), wherein the modified version has HPPD enzymatic activity and comprises the amino acid sequence set forth in SEQ ID NO: 59 (FEFMPSPPPTYY), wherein the first M is replaced with any other amino acid, particularly with I; and/or wherein the first P is replaced with any other amino acid, particularly with N; and/or wherein the first S is replaced with any other amino acid, particularly with V; and/or wherein the third P is replaced with any other amino acid, particularly with G or T; and/or wherein the fourth P is replaced with any other amino acid, particularly with G or removed.

According to some embodiments, the HPPD protein may be a modified version of the HPPD protein endogenous to G. max (Gm_HPPD), wherein the modified version has HPPD enzymatic activity and comprises the amino acid sequence set forth in SEQ ID NO: 60 (TYYANLHNRA), wherein the first A is replaced with any other amino acid, particularly with D; and/or wherein the first H is replaced with any other amino acid, particularly with R; and/or wherein the second N is replaced with any other amino acid, particularly with L.

According to some embodiments, the HPPD protein may be a modified version of the HPPD protein endogenous to G. max (Gm_HPPD), wherein the modified version has HPPD enzymatic activity and comprises the amino acid sequence set forth in SEQ ID NO: 61 (EELGILVD), wherein the first G is replaced with any other amino acid, particularly with N or D.

According to some embodiments, the HPPD protein may be a modified version of the HPPD protein endogenous to G. max (Gm_HPPD), wherein the modified version has HPPD enzymatic activity and comprises the amino acid sequence set forth in SEQ ID NO: 62 (VDRDDQGT), wherein the first R is replaced with any other amino acid, particularly with Y or F; and/or wherein the first Q is replaced with any other amino acid, particularly with A or G.

According to some embodiments, the HPPD protein may be a modified version of the HPPD protein endogenous to G. max (Gm_HPPD), wherein the modified version has HPPD enzymatic activity and comprises the amino acid sequence set forth in SEQ ID NO: 63 (EIIQRIGCM), wherein the third I is replaced with any other amino acid, particularly with N; and/or wherein the first G is replaced with any other amino acid, particularly with N; and/or wherein the first C is replaced with any other amino acid, particularly with F; and/or wherein the first M is replaced with any other amino acid, particularly with G or removed.

According to some embodiments, the HPPD protein may be a modified version of the HPPD protein endogenous to G. max (Gm_HPPD), wherein the modified version has HPPD enzymatic activity and comprises the amino acid sequence set forth in SEQ ID NO: 64 (CMVEDEEGK), wherein the first C is replaced with any other amino acid, particularly with F; and/or wherein all the amino acids but the first C are replaced with any other amino acid in particular with G or removed.

According to some embodiments, the HPPD protein may be a modified version of the HPPD protein endogenous to G. max (Gm_HPPD), wherein the modified version has HPPD enzymatic activity and comprises the amino acid sequence set forth in SEQ ID NO: 65 (GKVYQKGA), wherein all the amino acids are replaced with any other amino acid in particular with G or removed.

According to some embodiments, the HPPD protein may be a modified version of the HPPD protein endogenous to G. max (Gm_HPPD), wherein the modified version has HPPD enzymatic activity and comprises the amino acid sequence set forth in SEQ ID NO: 66 (YQKGACGGFG), wherein the first six amino acids are replaced with any other amino acid in particular with G or removed; and/or wherein the second G is replaced with any other amino acid, particularly with E or S.

According to some embodiments, the HPPD protein may be a modified version of the HPPD protein endogenous to G. max (Gm_HPPD), wherein the modified version has HPPD enzymatic activity and comprises the amino acid sequence set forth in SEQ ID NO: 67 (AEVRLYGDV), wherein the first E is replaced with any other amino acid, particularly with T or V.

According to some embodiments, the HPPD protein may be a modified version of the HPPD protein endogenous to G. max (Gm_HPPD), wherein the modified version has HPPD enzymatic activity and comprises the amino acid sequence set forth in SEQ ID NO: 68 (DVVLRYVSY), wherein the first Y is replaced with any other amino acid, particularly with F.

According to some embodiments, the HPPD protein may be a modified version of the HPPD protein endogenous to G. max (Gm_HPPD), wherein the modified version has HPPD enzymatic activity and comprises the amino acid sequence set forth in SEQ ID NO: 69 (ELAPAVR), wherein the first P is replaced with any other amino acid, particularly with S or K.

According to some embodiments, the HPPD protein may be a modified version of the HPPD protein endogenous to G. max (Gm_HPPD), wherein the modified version has HPPD enzymatic activity and comprises the amino acid sequence set forth in SEQ ID NO: 70 (VRYLKGF), wherein the first Y is replaced with any other amino acid, particularly with F.

According to some embodiments, the HPPD protein may be a modified version of the HPPD protein endogenous to G. max (Gm_HPPD), wherein the modified version has HPPD enzymatic activity and comprises the amino acid sequence set forth in SEQ ID NO: 71 (GFGKGNF), wherein the first K is replaced with any other amino acid, particularly with A.

According to some embodiments, the HPPD protein may be a modified version of the HPPD protein endogenous to G. max (Gm_HPPD), wherein the modified version has HPPD enzymatic activity and comprises the amino acid sequence set forth in SEQ ID NO: 72 (FVRTNP), wherein the first R is replaced with any other amino acid, particularly with A, D or Q.

According to some embodiments, the HPPD protein may be a modified version of an endogenous HPPD protein of a dicot plant, wherein a motif of the endogenous HPPD protein has the consensus sequence set forth in SEQ ID NO: 73 ((E,R)XAF(S,T)(A,I,T)SVX(K,N,H)GA—wherein X may be any amino acid) and, wherein the modified version has HPPD enzymatic activity and comprises the amino acid sequence set forth in SEQ ID NO: 73, wherein the F at the forth position is replaced with any other amino acid, particularly with Y, as set forth in SEQ ID NO: 119 ((E,R)XAY(S,T)(A,I,T)SVX(K,N,H)GA); and/or wherein the amino acid (S,T) at the fifth position is replaced with any other amino acid, particularly with N, as set forth in SEQ ID NO: 120((E,R)XAFN(A,I,T)SVX(K,N,H)GA); and/or wherein the amino acid (A,I,T) at the sixth position is replaced with any other amino acid, particularly with K, as set forth in SEQ ID NO: 121 ((E,R)XAF(S,T)KSVX(K,N,H)GA); and/or wherein the amino acid S at the seventh position is replaced with any other amino acid, particularly with A, as set forth in SEQ ID NO: 122 ((E,R)XAF(S,T)(A,I,T)AVX(K,N,H)GA); and/or wherein the amino acid (K,N,H) at the tenth position is replaced with any other amino acid, particularly with E, as set forth in SEQ ID NO: 123 ((E,R)XAF(S,T)(A,I,T)SVXEGA).

According to some embodiments, the HPPD protein may be a modified version of an endogenous HPPD protein of a dicot plant, wherein a motif of the endogenous HPPD protein has the consensus sequence set forth in SEQ ID NO: 74 (VX(K,N,H)GAEP(A,S)S(P,E)P)—wherein X may be any amino acid) and, wherein the modified version has HPPD enzymatic activity and comprises the amino acid sequence set forth in SEQ ID NO: 74, wherein the amino acid (K,N,H) at the third position is replaced with any other amino acid, particularly with E, as set forth in SEQ ID NO: 124 (VXEGAEP(A,S)S(P,E)P); and/or wherein the amino acid E at the sixth position is replaced with any other amino acid, particularly with I, as set forth in SEQ ID NO: 125 (VX(K,N,H)GAIP(A,S)S(P,E)P); and/or wherein the amino acid P at the seventh position is replaced with any other amino acid, particularly with A, as set forth in SEQ ID NO: 126 (VX(K,N,H)GAEA(A,S)S(P,E)P); and/or wherein the amino acid S at the ninth position is replaced with any other amino acid, particularly with Q, as set forth in SEQ ID NO: 127 (VX(K,N,H)GAEP(A,S)Q(P,E)P); and/or wherein the amino acid (P,E) at the tenth position is replaced with any other amino acid, particularly with G, as set forth in SEQ ID NO: 128 (VX(K,N,H)GAEP(A,S)SGP).

According to some embodiments, the HPPD protein may be a modified version of an endogenous HPPD protein of a dicot plant, wherein a motif HPPD protein has the consensus sequence set forth in SEQ ID NO: 75 (FH(E,Q)FAEFT(A,T)) and, wherein the modified version of the endogenous has HPPD enzymatic activity and comprises the amino acid sequence set forth in SEQ ID NO: 75, wherein the amino acid A at the fifth position is replaced with any other amino acid, particularly with W or R, as set forth in SEQ ID NO: 129 (FH(E,Q)F(W,R)EFT(A,T)); and/or wherein the amino acid T at the eight position is replaced with any other amino acid, particularly with D, as set forth in SEQ ID NO: 130 (FH(E,Q)FAEFD(A,T)); and/or wherein the amino acid (A,T) at the tenth position is replaced with any other amino acid, particularly with D, as set forth in SEQ ID NO: 131 (FH(E,Q)FAEFTD).

According to some embodiments, the HPPD protein may be a modified version of an endogenous HPPD protein of a dicot plant, wherein a motif HPPD protein has the consensus sequence set forth in SEQ ID NO: 76 (T(A,T)(E,D)DVGT(A,S)ES) and, wherein the modified version of the endogenous has HPPD enzymatic activity and comprises the amino acid sequence set forth in SEQ ID NO: 76, wherein the amino acid T at the first position is replaced with any other amino acid, particularly with D, as set forth in SEQ ID NO: 132 (D(A,T)(E,D)DVGT(A,S)ES); and/or wherein the amino acid (A,T) at the second position is replaced with any other amino acid, particularly with D, as set forth in SEQ ID NO: 133 (TD(E,D)DVGT(A,S)ES); and/or wherein the amino acid E at the ninth position is replaced with any other amino acid, particularly with F or Y, as set forth in SEQ ID NO: 134 (T(A,T)(E,D)DVGT(A,S)(F,Y)S).

According to some embodiments, the HPPD protein may be a modified version of an the consensus sequence set forth in SEQ ID NO: 77 (F(E,D)FMPSPP(P,V)TYY) and, wherein the modified version has HPPD enzymatic activity and comprises the amino acid sequence set forth in SEQ ID NO: 77, wherein the amino acid M at the fourth position is replaced with any other amino acid, particularly with I, as set forth in SEQ ID NO: 135 (F(E,D)FIPSPP(P,V)TYY); and/or wherein the amino acid P at the fifth position is replaced with any other amino acid, particularly with N, as set forth in SEQ ID NO: 136 (F(E,D)FMNSPP(P,V)TYY); and/or wherein the amino acid S at the sixth position is replaced with any other amino acid, particularly with V, as set forth in SEQ ID NO: 137 (F(E,D)FMPVPP(P,V)TYY); and/or wherein the amino acid P at the eighth position is replaced with any other amino acid, particularly with G or T, as set forth in SEQ ID NO: 138 (F(E,D)FMPSP(G,T)(P,V)TYY); and/or wherein the amino acid (P,V) at the ninth position is replaced with any other amino acid, particularly with G or removed, as set forth in SEQ ID NO: 139 (F(E,D)FMPSPPGTYY) and SEQ ID NO: 140 (F(E,D)FMPSPPTYY), respectively.

According to some embodiments, the HPPD protein may be a modified version of an endogenous HPPD protein of a dicot plant, wherein a motif of the endogenous HPPD protein has the consensus sequence set forth in SEQ ID NO: 78 (TYYX(N,K)L(H,K,R)X(A,G)L—wherein X may be any amino acid) and, wherein the modified version has HPPD enzymatic activity and comprises the amino acid sequence set forth in SEQ ID NO: 78, wherein the amino acid at the fourth position is replaced with any other amino acid, particularly with D, as set forth in SEQ ID NO: 141(TYYD(N,K)L(H,K,R)X(A,G)L); and/or wherein the amino acid at the eighth position is replaced with any other amino acid, particularly with L, as set forth in SEQ ID NO: 142 (TYYX(N,K)L(H,K,R)L(A,G)L).

According to some embodiments, the HPPD protein may be a modified version of an endogenous HPPD protein of a dicot plant, wherein a motif of the endogenous HPPD protein has the consensus sequence set forth in SEQ ID NO: 79 (VD(R,K)DDQGT) and, wherein the modified version has HPPD enzymatic activity and comprises the amino acid sequence set forth in SEQ ID NO: 79, wherein the amino acid (R,K) at the third position is replaced with any other amino acid, particularly with For Y, as set forth in SEQ ID NO: 143 (VD(F,Y)DDQGT); and/or wherein the amino acid Q at the sixth position is replaced with any other amino acid, particularly with A or G, as set forth in SEQ ID NO: 144 (VD(R,K)DD(A,G)GT).

According to some embodiments, the HPPD protein may be a modified version of an endogenous HPPD protein of a dicot plant, wherein a motif of the endogenous HPPD protein has the consensus sequence set forth in SEQ ID NO: 80 (EIIQR(I,V)GCM) and, wherein the modified version has HPPD enzymatic activity and comprises the amino acid sequence set forth in SEQ ID NO: 80, wherein the amino acid amino acid (I,V) at the sixth position is replaced with any other amino acid, particularly with N, as set forth in SEQ ID NO: 145 (EIIQRNGCM); and/or wherein the amino acid G at the seventh position is replaced with any other amino acid, particularly with N, as set forth in SEQ ID NO: 146 (EIIQR(I,V)NCM); and/or wherein the amino acid C at the eighth position is replaced with any other amino acid, particularly with F, as set forth in SEQ ID NO: 147 (EIIQR(I,V)GFM); and/or wherein the amino acid M at the ninth position is replaced with any other amino acid, particularly with G or removed, as set forth in SEQ ID NO: 148 (EIIQR(I,V)GCG) and SEQ ID NO: 149 (EIIQR(I,V)GC), respectively.

According to some embodiments, the HPPD protein may be a modified version of an endogenous HPPD protein of a dicot plant, wherein a motif of the endogenous HPPD protein has the consensus sequence set forth in SEQ ID NO: 81 (CMX(E,K,Q)(D,N)(E,A)EG(K,E)—wherein X may be any amino acid) and, wherein the modified version has HPPD enzymatic activity and comprises the amino acid sequence set forth in SEQ ID NO: 81, wherein the amino acid amino acid C at the first position is replaced with any other amino acid, particularly with F, as set forth in SEQ ID NO: 150 (FMX(E,K,Q)(D,N)(E,A)EG(K,E)); and/or all the amino acids but the amino acid C at the first position are replaced with any other amino acid, particularly with G or removed.

According to some embodiments, the HPPD protein may be a modified version of an endogenous HPPD protein of a dicot plant, wherein a motif of the endogenous HPPD protein has the consensus sequence set forth in SEQ ID NO: 82 (G(K,E)XYQ(K,S)G(G,A)—wherein X may be any amino acid) and, wherein the modified version has HPPD enzymatic activity and comprises the amino acid sequence set forth in SEQ ID NO: 82, all the amino acids are replaced with any other amino acid, particularly with G or removed.

According to some embodiments, the HPPD protein may be a modified version of an the consensus sequence set forth in SEQ ID NO: 83 (YQ(K,S)G(G,A)CGGFG) and, wherein the modified version has HPPD enzymatic activity and comprises the amino acid sequence set forth in SEQ ID NO: 83, wherein the first six amino acids are replaced with any other amino acid, particularly with G or removed; and/or wherein the amino acid G at the seventh position is replaced with any other amino acid, particularly with E or S, as set forth in SEQ ID NO: 151 (YQ(K,S)G(G,A)C(E,S)GFG).

According to some embodiments, the HPPD protein may be a modified version of an endogenous HPPD protein of a dicot plant, wherein a motif of the endogenous HPPD protein has the consensus sequence set forth in SEQ ID NO: 84 ((A,S)EV(R,H,K)LYGDV) and, wherein the modified version has HPPD enzymatic activity and comprises the amino acid sequence set forth in SEQ ID NO: 84, wherein the amino acid E at the second position is replaced with any other amino acid, particularly with T or V, as set forth in SEQ ID NO: 152 ((A,S)(T,V)V(R,H,K)LYGDV).

According to some embodiments, the HPPD protein may be a modified version of an endogenous HPPD protein of a dicot plant, wherein a motif of the endogenous HPPD protein has the consensus sequence set forth in SEQ ID NO: 85 ((E,Q)L(A,G,S)P(A,V)(V,L,I)X—wherein X may be any amino acid) and, wherein the modified version has HPPD enzymatic activity and comprises the amino acid sequence set forth in SEQ ID NO: 85, wherein the amino acid P at the fourth position is replaced with any other amino acid, particularly with S or K, as set forth in SEQ ID NO: 153 ((E,Q)L(A,G,S)(S,K)(A,V)(V,L,I)X).

According to some embodiments, the HPPD protein may be a modified version of an endogenous HPPD protein of a dicot plant, wherein a motif of the endogenous HPPD protein has the consensus sequence set forth in SEQ ID NO: 86 ((V,L,I)XY(L,V,I)(K,A)XF—wherein X may be any amino acid) and, wherein the modified version has HPPD enzymatic activity and comprises the amino acid sequence set forth in SEQ ID NO: 86, wherein the amino acid Y at the third position is replaced with any other amino acid, particularly with F, as set forth in SEQ ID NO: 154 ((V,L,I)XF(L,V,I)(K,A)XF).

According to some embodiments, the HPPD protein may be a modified version of an the consensus sequence set forth in SEQ ID NO: 87 (F(V,I)RXNP—wherein X may be any amino acid) and, wherein the modified version has HPPD enzymatic activity and comprises the amino acid sequence set forth in SEQ ID NO: 87, wherein the amino acid R at the third position is replaced with any other amino acid, particularly with A, D or Q, as set forth in SEQ ID NO: 155 (F(V,I)(A,D,Q)XNP).

According to some embodiments, the HPPD protein may be a modified version of an endogenous HPPD protein of a dicot plant, wherein a motif of the endogenous HPPD protein has the consensus sequence set forth in SEQ ID NO: 199 (EELGILVD), wherein the first G is replaced with any other amino acid, particularly with N or D, as set forth in SEQ ID NO: 156 (EEL(N,D)ILVD).

According to some embodiments, the HPPD protein may be a modified version of an endogenous HPPD protein of a dicot plant, wherein a motif of the endogenous HPPD protein has the consensus sequence set forth in SEQ ID NO: 200 (DVVLRYVS), wherein the first Y is replaced with any other amino acid, particularly with F, as set forth in SEQ ID NO: 157 (DVVLRFVS).

According to some embodiments, the HPPD protein may be a modified version of an endogenous HPPD protein of a dicot plant, wherein a motif of the endogenous HPPD protein has the consensus sequence set forth in SEQ ID NO: 201 (GFGKGNF), wherein the first K is replaced with any other amino acid, particularly with A, as set forth in SEQ ID NO: 158 (GFGAGNF).

According to some embodiments, the HPPD protein may be a modified version of an endogenous HPPD protein of a monocot plant, wherein a motif of the endogenous HPPD protein has the consensus sequence set forth in SEQ ID NO: 88 ((E,A)(D,E)AFR(A,V)SVA(A,G)GA) and, wherein the modified version has HPPD enzymatic activity and comprises the amino acid sequence set forth in SEQ ID NO: 88, wherein the amino acid F at the fourth position is replaced with any other amino acid, particularly with Y, as set forth in SEQ ID NO: 159 ((E,A)(D,E)AYR(A,V)SVA(A,G)GA); and/or wherein the amino acid R at the fifth position is replaced with any other amino acid, particularly with N, as set forth in SEQ ID NO: 160 ((E,A)(D,E)AFN(A,V)SVA(A,G)GA); and/or wherein the amino acid (A,V) at the sixth position is replaced with any other amino acid, particularly with K, as set forth in SEQ ID NO: 161 ((E,A)(D,E)AFRKSVA(A,G)GA); and/or wherein the amino acid S at the seventh position is replaced with any other amino acid, particularly with A, as set forth in SEQ ID NO: 162 ((E,A)(D,E)AFR(A,V)SVA(A,G)GA); and/or wherein the amino acid (A,G) at the tenth position is replaced with any other amino acid, particularly with E, as set forth in SEQ ID NO: 163 ((E,A)(D,E)AFR(A,V)SVAEGA).

According to some embodiments, the HPPD protein may be a modified version of an endogenous HPPD protein of a monocot plant, wherein a motif of the endogenous HPPD protein has the consensus sequence set forth in SEQ ID NO: 89 (VA(A,G)GARPAF(Q,A)P) and, wherein the modified version has HPPD enzymatic activity and comprises the amino acid sequence set forth in SEQ ID NO: 89, wherein the amino acid (A,G) at the third position is replaced with any other amino acid, particularly with E, as set forth in SEQ ID NO: 164 (VAEGARPAF(Q,A)P); and/or wherein the amino acid R at the sixth position is replaced with any other amino acid, particularly with I, as set forth in SEQ ID NO: 165 (VA(A,G)GAIPAF(Q,A)P); and/or wherein the amino acid P at the seventh position is replaced with any other amino acid, particularly with A, as set forth in SEQ ID NO: 166 (VA(A,G)GARAAF(Q,A)P); and/or wherein the amino acid F at the ninth position is replaced with any other amino acid, particularly with Q, as set forth in SEQ ID NO: 167 (VA(A,G)GARPAQ(Q,A)P); and/or wherein the amino acid (Q,A) at the tenth position is replaced with any other amino acid, particularly with G, as set forth in SEQ ID NO: 168 (VA(A,G)GARPAFGP).

According to some embodiments, the HPPD protein may be a modified version of an endogenous HPPD protein of a monocot plant, wherein a motif of the endogenous HPPD protein has the consensus sequence set forth in SEQ ID NO: 90 (FHEFAEFT(A,T)) and, wherein the modified version has HPPD enzymatic activity and comprises the amino acid sequence set forth in SEQ ID NO: 90, wherein the amino acid A at the fifth position is replaced with any other amino acid, particularly with W or R, as set forth in SEQ ID NO: 169 (FHEF(W,R)EFT(A,T)); and/or wherein the amino acid T at the eighth position is replaced with any other amino acid, particularly with D, as set forth in SEQ ID NO: 170 (FHEFAEFD(A,T)); and/or wherein the amino acid (A,T) at the tenth position is replaced with any other amino acid, particularly with D, as set forth in SEQ ID NO: 171 (FHEFAEFTD).

According to some embodiments, the HPPD protein may be a modified version of an endogenous HPPD protein of a monocot plant, wherein a motif of the endogenous HPPD protein has the consensus sequence set forth in SEQ ID NO: 91 (T(A,T)EDVGT(A,T)ES) and, wherein the modified version has HPPD enzymatic activity and comprises the amino acid sequence set forth in SEQ ID NO: 91, wherein the amino acid T at the first position is replaced with any other amino acid, particularly with D, as set forth in SEQ ID NO: 172 (D(A,T)EDVGT(A,T)ES); and/or wherein the amino acid (A,T) at the second position is replaced with any other amino acid, particularly with D, as set forth in SEQ ID NO: 173 (TDEDVGT(A,T)ES); and/or wherein the amino acid E at the ninth position is replaced with any other amino acid, particularly with For Y, as set forth in SEQ ID NO: 174 (T(A,T)EDVGT(A,T)(F,Y)S).

According to some embodiments, the HPPD protein may be a modified version of an endogenous HPPD protein of a monocot plant, wherein a motif of the endogenous HPPD protein has the consensus forth sequence set in SEQ ID NO: 92 (FEF(L,M)APP(P,T,Q)(S,P,A)(D,N,K)YYW) and, wherein the modified version has HPPD enzymatic activity and comprises the amino acid sequence set forth in SEQ ID NO: 92, wherein the amino acid (L,M) at the fourth position is replaced with any other amino acid, particularly with I, as set forth in SEQ ID NO: 175 (FEFIAPP(P,T,Q)(S,P,A)(D,N,K)YYW); and/or wherein the amino acid A at the fifth position is replaced with any other amino acid, particularly with N, as set forth in SEQ ID NO: 176 (FEF(L,M)NPP(P,T,Q)(S,P,A)(D,N,K)YYW); and/or wherein the amino acid P at the sixth position is replaced with any other amino acid, particularly with V, as set forth in SEQ ID NO: 177 (FEF(L,M)AVP(P,T,Q)(S,P,A)(D,N,K)YYW); and/or wherein the amino acid (P,T,Q) at the eighth position is replaced with any other amino acid, particularly with G, as set forth in SEQ ID NO: 178 (FEF(L,M)APPG(S,P,A)(D,N,K)YYW); and/or wherein the amino acid (S,P,A) at the ninth position is replaced with any other amino acid, particularly with G or removed, as set forth in SEQ ID NO: 179 (FEF(L,M)APP(P,T,Q)G(D,N,K)YYW) and SEQ ID NO: 180 (FEF(L,M)APP(P,T,Q)(D,N,K)YYW), respectively.

According to some embodiments, the HPPD protein may be a modified version of an endogenous HPPD protein of a monocot plant, wherein a motif of the endogenous HPPD protein has the consensus sequence set forth in SEQ ID NO: 93 ((D,N,K)YY(D,E)GVRRGV) and, wherein the modified version has HPPD enzymatic activity and comprises the amino acid sequence set forth in SEQ ID NO: 93, wherein the amino acid (D, E) at the fourth position is replaced with any other amino acid, particularly with D, as set forth in SEQ ID NO: 181 ((D,N,K)YYDGVRRGV); and/or wherein the amino acid R at the eighth position is replaced with any other amino acid, particularly with L, as set forth in SEQ ID NO: 182 ((D,N,K)YY(D,E)GVRLGV).

According to some embodiments, the HPPD protein may be a modified version of an endogenous HPPD protein of a monocot plant, wherein a motif of the endogenous HPPD protein has the consensus sequence set forth in SEQ ID NO: 94 (QELGVLVD) and, wherein the modified version has HPPD enzymatic activity and comprises the amino acid sequence set forth in SEQ ID NO: 94, wherein the first G is replaced with any other amino acid, particularly with N or D, as set forth in SEQ ID NO: 183 (QEL(N,D)VLVD).

According to some embodiments, the HPPD protein may be a modified version of an endogenous HPPD protein of a monocot plant, wherein a motif of the endogenous HPPD protein has the consensus sequence set forth in SEQ ID NO: 95 (VDRDDQGV) and wherein the modified version has HPPD enzymatic activity and comprises the amino acid sequence set forth in SEQ ID NO: 95, wherein the first R is replaced with any other amino acid, particularly with Y or F, as set forth in SEQ ID NO: 184 (VD(Y,F)DDQGV); and/or wherein the first Q is replaced with any other amino acid, particularly with A or G, as set forth in SEQ ID NO: 185 (VDRDD(A,G)GV).

According to some embodiments, the HPPD protein may be a modified version of an endogenous HPPD protein of a monocot plant, wherein a motif of the endogenous HPPD protein has the consensus sequence set forth in SEQ ID NO: 96 (EMIQRIGCM) and wherein the modified version has HPPD enzymatic activity and comprises the amino acid sequence set forth in SEQ ID NO: 96, wherein the amino acid I at the sixth position is replaced with any other amino acid, particularly with N, as set forth in SEQ ID NO: 186 (EMIQRNGCM); and/or wherein the amino acid G at the seventh position is replaced with any other amino acid, particularly with N, as set forth in SEQ ID NO: 187 (EMIQRINCM); and/or wherein the amino acid C at the eighth position is replaced with any other amino acid, particularly with F, as set forth in SEQ ID NO: 188 (EMIQRIGFM); and/or wherein the amino acid M at the ninth position is replaced with any other amino acid, particularly with G or removed, as set forth in SEQ ID NO: 189 (EMIQRIGCG) and SEQ ID NO: 190 (EMIQRIGC).

According to some embodiments, the HPPD protein may be a modified version of an endogenous HPPD protein of a monocot plant, wherein a motif of the endogenous HPPD protein has the consensus sequence set forth in SEQ ID NO: 97 (CMEKDE(K,S,V)GQ) and wherein the modified version has HPPD enzymatic activity and comprises the amino acid sequence set forth in SEQ ID NO: 97, wherein the amino acid C at the first position is replaced with any other amino acid, particularly with F, as set forth in SEQ ID NO: 191 (FMEKDE(K,S,V)GQ); and/or wherein all the amino acids but the amino acid C at the first position are replaced with any other amino acid, particularly with G or removed.

According to some embodiments, the HPPD protein may be a modified version of an endogenous HPPD protein of a monocot plant, wherein a motif of the endogenous HPPD protein has the consensus sequence set forth in SEQ ID NO: 98 (GQEYQKGA) and wherein the modified version has HPPD enzymatic activity and comprises the amino acid sequence set forth in SEQ ID NO: 98, wherein all the amino acids are replaced with any other amino acid, particularly with G or removed.

According to some embodiments, the HPPD protein may be a modified version of an endogenous HPPD protein of a monocot plant, wherein a motif of the endogenous HPPD protein has the consensus sequence set forth in SEQ ID NO: 99 (AEVELYGDV) and, wherein the modified version has HPPD enzymatic activity and comprises the amino acid sequence set forth in SEQ ID NO: 99, wherein the amino acid E at the second position is replaced with any other amino acid, particularly with T or V, as set forth in SEQ ID NO: 192 (A(T,V)VELYGDV).

According to some embodiments, the HPPD protein may be a modified version of an endogenous HPPD protein of a monocot plant, wherein a motif of the endogenous HPPD protein has the consensus sequence set forth in SEQ ID NO: 100 (E(L,M)AP(A,V)(A,I)(A,D)) and, wherein the modified version has HPPD enzymatic activity and comprises the amino acid sequence set forth in SEQ ID NO: 100, wherein the amino acid P at the fourth position is replaced with any other amino acid, particularly with S or K, as set forth in SEQ ID NO: 193 (E(L,M)A(S,K)(A,V)(A,I)(A,D)).

According to some embodiments, the HPPD protein may be a modified version of an endogenous HPPD protein of a monocot plant, wherein a motif of the endogenous HPPD protein has the consensus sequence set forth in SEQ ID NO: 101 ((A,I)(A,D)Y(F,I,M)(A,S,K)GF) and wherein the modified version has HPPD enzymatic activity and comprises the amino acid sequence set forth in SEQ ID NO: 101, wherein the amino acid Y at the third position is replaced with any other amino acid, particularly with F, as set forth in SEQ ID NO: 194 ((A,I)(A,D)F(F,I,M)(A,S,K)GF).

According to some embodiments, the HPPD protein may be a modified version of an endogenous HPPD protein of a monocot plant, wherein a motif of the endogenous HPPD protein has the consensus sequence set forth in SEQ ID NO: 102 ((F,V)VR(F,A,V)NP) and wherein the modified version has HPPD enzymatic activity and comprises the amino acid sequence set forth in SEQ ID NO: 102, wherein the amino acid R at the third position is replaced with any other amino acid, particularly with A, D or Q, as set forth in SEQ ID NO: 195 ((F,V)V(A,D,Q)(F,A,V)NP).

According to some embodiments, the HPPD protein may be a modified version of an endogenous HPPD protein of a monocot plant, wherein a motif of the endogenous HPPD protein has the consensus sequence set forth in SEQ ID NO: 202 (YQKGACGGFG), wherein the first six amino acids are replaced with any other amino acid in particular with G or removed; and/or wherein the second G is replaced with any other amino acid, particularly with E or S, as set forth in SEQ ID NO: 196 (YQKGAC(E,S)GFG).

According to some embodiments, the HPPD protein may be a modified version of an endogenous HPPD protein of a monocot plant, wherein a motif of the endogenous HPPD protein has the consensus sequence set forth in SEQ ID NO: 203 (DVVLRYVSY), wherein the first Y is replaced with any other amino acid, particularly with F, as set forth in SEQ ID NO: 197 (DVVLRFVSY).

According to some embodiments, the HPPD protein may be a modified version of an endogenous HPPD protein of a monocot plant, wherein a motif of the endogenous HPPD protein has the consensus sequence set forth in SEQ ID NO: 204 (GFGKGNF), wherein the first K is replaced with any other amino acid, particularly with A, as set forth in SEQ ID NO: 198 (GFGAGNF).

According to some embodiments, the polypeptides of the invention are catalytically active HPPDs derived from a fungus that confer efficient levels of resistance and/or tolerance to certain classes of herbicides that inhibit HPPD. According to some embodiments, the HPPD, and sequence encoding same, is derived from a fungus of the species Trichoderma. According to some embodiments, the HPPD, and sequence encoding same, is derived from a fungus of the species Trichoderma. According to some embodiments, the HPPD, and sequence encoding same, is derived from a fungus of the species T. asperellum, T. harzianum, T. viride, T. hamatum or any combination thereof. According to some embodiments, the HPPD, and sequence encoding same, is derived from a fungus of the species T. harzianum. According to some embodiments, the HPPD, is derived from the Trichoderma Ti-123 disclosed in U.S. 63/018,027. According to some embodiments, the HPPD, and sequence encoding same, is derived from a fungus of the species Talaromyces spp.

Exemplary HPPD polypeptides according to the invention correspond to the amino acid sequences set forth in SEQ ID NO: 1, 3, 5-8 and variants and fragments thereof. Nucleic acid molecules comprising polynucleotide sequences that encode these particular HPPD polypeptides are set forth in SEQ ID NO: 2, 4 although other nucleic acid sequences may also be envisaged. Compositions also include expression cassettes comprising a promoter operably linked to a nucleotide sequence that encodes an HPPD polypeptide of the invention, alone or in combination with one or more additional nucleic acid molecules encoding polypeptides that confer desirable traits. Transformed plants, plant cells, and seeds comprising an expression cassette of the invention are further provided.

The compositions of the invention are useful in methods directed to conferring herbicide resistance or tolerance to plants, particularly resistance or tolerance to certain classes of herbicides that inhibit HPPD. In particular embodiments, the methods comprise introducing into a plant at least one expression cassette comprising a promoter operably linked to a nucleotide sequence that encodes an HPPD polypeptide of the invention. As a result, the HPPD polypeptide is expressed in the plant, and since the HPPD is selected on the basis that it is less sensitive to HPPD-inhibiting herbicides, this leads to the plant exhibiting substantially improved resistance or tolerance to HPPD-inhibiting herbicides.

Methods of the present invention also comprise selectively controlling weeds in a crop field. In one embodiment, such methods involve over-the-top pre- or postemergence application of weed-controlling amounts of HPPD herbicides in a field that contains plants expressing the HPPD polypeptides of the invention. In other embodiments, methods are also provided for the assay, characterization, identification, and selection of the HPPDs of the current invention.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The invention will now be described in relation to certain examples and embodiments with reference to the following illustrative figures so that it may be more fully understood.

FIG. 1 shows illustrative images obtained from a HPPD-activity screen of E. coli BL21 (DE3) transformed with a) a HPPD cDNA derived from a Trichoderma spp. (as disclosed herein), b) a HPPD cDNA derived from Arabidopsis thaliana, and c) empty vector when grown in increasing concentrations HPPD inhibitors.

FIG. 2 shows illustrative images of Arabidopsis TI plants transformed to express the herein disclosed Trichoderma spp-HPPD gene as compared to control (non-transformed), when grown on Basta and treated with 0.1% or 0.05% Tembotrione.

FIG. 3A and FIG. 3B show illustrative images of Arabidopsis T2 plants transformed to express the herein disclosed Trichoderma spp-HPPD gene as compared to control (wild type (wt) non-transformed), when grown on Basta and treated with Tembotrione x1, Tembotrione x0.2 and Mesotrione x1 (FIG. 3A) and Tembotrione x1, Topramezon x0.2, Topramezon x1, Isoxaflutole x1 (FIG. 3B).

FIG. 4 shows the alignment of twelve different HPPD sequences. The positions of secondary structural elements are shown on top of the alignment. Motifs 1-9 are framed by dashed line boxes. Motif numbers are indicated by triangles and corresponding numbers. Structural elements are indicated by boxes/arrows above the sequences: H indicating a-helix, A-D indicating B-sheets. Mutations designed for soy HPPD are framed by solid line boxes over the soy sequence.

FIG. 5A shows a 3D model of molecular interactions between motifs 3, 5 and 8 in HPPD of A. thaliana;

FIG. 5B shows the predicted 3D structure of the HPPD set forth in SEQ ID NO: 1, built based on the 3D structure of FIG. 5A.

FIG. 6A shows a 3D model of molecular interactions between motifs 2, 4 and 9 in HPPD of A. thaliana.

FIG. 6B shows the predicted 3D structure of part of the HPPD set forth in SEQ ID NO: 1.

FIG. 7A shows the influence of motif 5 on the loop and helix 9 (H9) of the HPPD of A. thaliana.

FIG. 7B shows the influence of motif 5 on the loop and helix 9 (H9) of HPPD built based on SEQ ID NO: 1.

FIG. 7C, shows the superimposition of FIG. 7A and FIG. 7B.

FIG. 8 schematically illustrates chimeric constructs of the N-terminal sequence of the herein disclosed fungal HPPD of truncated HPPD (deletion of amino acids 1-22 (A22) or of amino acids 1-38 (A38)), optionally fused to a plant derived N-terminal chloroplast transit peptide (cTP) sequence.

FIG. 9 shows illustrative images obtained from a HPPD-activity screen of E. coli BL21 (DE3) transformed with the chimeric Trichoderma spp derived HPPD shown in FIG. 8 in the absence or presence of increasing concentrations of tembotrione (Laudis) HPPD inhibitor. A brown halo is indicative of HPPD activity.

FIG. 10 shows illustrative images obtained from a HPPD-activity screen of E. coli BL21 (DE3) transformed with Arabidopsis thaliana HPPD cDNA (accession no. AF047834). A brown halo is indicative of HPPD activity.

FIG. 11 shows illustrative images obtained from a HPPD-activity screen of E. coli BL21 (DE3) transformed with HPPD cDNA derived of mutated HPPD derived from G. max (GmHPPD=wt (Accession number: ABQ96868) set forth in SEQ ID NO: 118); GmHPPD.1=GmHPPD (SEQ ID NO: 118) mutated to substitute its motif 1 with motif 1 of SEQ ID NO: 1—i.e. to include amino acid sequences SEQ ID NO: 9-10 or 38; GmHPPD.2=GmHPPD (SEQ ID NO: 118) mutated to substitute its motif 6.2 (SEQ ID NO: 68) first Y with motif 6.2 of SEQ ID NO: 1 (SEQ ID NO: 20)—i.e. to include the mutation Tyr185Phe to include SEQ ID NO: 157; GmHPPD.4=GmHPPD (SEQ ID NO: 118) mutated to substitute its motif 2.1 (SEQ ID NO: 57) with motif 2.1 of SEQ ID NO: 1 (SEQ ID NO: 11)—i.e. to include the mutation Ala254Trp to include SEQ ID NO: 129 in the absence or presence of increasing concentrations of tembotrione (Laudis) HPPD inhibitor. A brown halo is indicative of HPPD activity.

FIG. 12 shows TI generation transformed seeds of Camelina plants with the Soy HPPD (GmHPPD) mutated gene as compared to wt and EGFP. HPPD derived from G. max (GmHPPD=wt (Accession number: ABQ96868) set forth in SEQ ID NO: 118); GmHPPD.1=GmHPPD (SEQ ID NO: 118) mutated to substitute its motif 1 with motif 1 of SEQ ID NO: 1—i.e. to include amino acid sequences SEQ ID NO: 9-10 or 38; GmHPPD.3=GmHPPD (SEQ ID NO: 118) mutated to substitute its motif 7.2 (SEQ ID NO: 70) first Y with motif 7.2 of SEQ ID NO: 1 (SEQ ID NO: 22)—i.e. to include the mutation Tyr243Phe to include SEQ ID NO: 154; GmHPPD.4=GmHPPD (SEQ ID NO: 118) mutated to substitute its motif 2.1 (SEQ ID NO: 57) with motif 2.1 of SEQ ID NO: 1 (SEQ ID NO: 11)—i.e. to include the mutation Ala254Trp to include SEQ ID NO: 129; GmHPPD.5=GmHPPD (SEQ ID NO: 118) mutated to substitute its motif 3.1 with motif 3.1 of SEQ ID NO: 1—i.e. to include the replacement mutations 338-MPSPPP-343 to INVPG to include amino acid sequences SEQ ID NO: 29.

### DETAILED DESCRIPTION

The present invention provides compositions and methods directed to conferring hydroxyphenyl pyruvate dioxygenase (HPPD) herbicide resistant and/or tolerant plants.

Compositions include amino acid sequences for HPPD polypeptides having fungus derived HPPD enzymatic activity, and variants and fragments thereof. Nucleic acids that encode the fungus derived HPPD polypeptides of the invention are also provided. Methods for conferring herbicide resistance and/or tolerance to plants, particularly resistance and/or tolerance to certain classes of herbicides that inhibit HPPD, are further provided. Methods are also provided for selectively controlling weeds in a crop field and for the assay, characterization, identification and selection of the mutant HPPDs of the current invention that provide herbicide tolerance.

Within the context of the present invention, the terms “hydroxy phenyl pyruvate dioxygenase (HPPD)”, “4-hydroxy phenyl pyruvate dioxygenase (4-HPPD)” and “p-hydroxy phenyl pyruvate dioxygenase (p-HPPD)” are synonymous.

“HPPD herbicides” are herbicides that are bleachers and whose primary site of action is HPPD. Many are well known and described elsewhere e.g. (Hawkes “Hydroxyphenylpyruvate Dioxygenase (HPPD)—The Herbicide Target.” In Modern Crop Protection Compounds. Eds. Kramer and Schirmer). As used herein, the term “HPPD herbicides” refers to herbicides that act either directly or indirectly to inhibit HPPD, where the herbicides are bleachers and where inhibition of HPPD is at least part of the herbicide's mode of action on plants.

As used herein, plants which are substantially “tolerant” to a herbicide exhibit, when treated with said herbicide, a dose/response curve which is shifted to the right when compared with that exhibited by similarly subjected non-tolerant plants. Tolerant plants will typically require at least twice as much herbicide as non-tolerant plants in order to produce a given herbicidal effect. Plants which are substantially “resistant” to the herbicide exhibit few, if any, necrotic, lytic, chlorotic or other lesions or, at least, none that impact significantly on yield, when subjected to the herbicide at concentrations and rates which are typically employed by the agricultural community to kill weeds in the field.

As used herein, the term “confer” refers to providing a characteristic or trait, such as herbicide tolerance or resistance, to a plant.

The terms “polypeptide”, “peptide”, and “protein” are used interchangeably herein to refer to a polymer of amino acid residues. The terms apply to amino acid polymers in which one or more amino acid residues is an artificial chemical analogue of a corresponding naturally occurring amino acid, as well as to naturally occurring amino acid polymers. Polypeptides of the invention can be produced either from a nucleic acid disclosed herein, or by the use of standard molecular biology techniques. For example, a truncated protein of the invention can be produced by expression of a recombinant nucleic acid of the invention in an appropriate host cell, or alternatively by a combination of ex-vivo procedures, such as protease digestion and purification.

As used herein, “nucleic acid” includes reference to a deoxyribonucleotide or ribonucleotide polymer in either single- or double-stranded form, and unless otherwise limited, encompasses known analogues (e.g., peptide nucleic acids) having the essential nature of natural nucleotides in that they hybridize to single-stranded nucleic acids in a manner similar to naturally occurring nucleotides.

Fragments and variants of the disclosed nucleotide sequences and proteins encoded thereby are also encompassed by the present invention. As used herein, “fragment” is intended to mean a portion of the nucleotide sequence or a portion of the amino acid sequence and hence protein encoded thereby. Fragments of a nucleotide sequence may encode protein fragments that retain the biological activity of the fungus-derived HPPD protein and hence have HPPD enzymatic activity. A fragment of a nucleotide sequence that encodes a biologically active portion of the fungus-derived HPPD protein of the invention will encode at least 15, 25, 30, 40, 50, 60, 70, 80, 90, 100, 110, 120, 150, 180, 200, 250, 300, 350 contiguous amino acids, or up to the total number of amino acids present in a full-length fungus-derived HPPD polypeptide of the invention. As used herein, “full-length sequence” in reference to a specified polynucleotide means having the entire nucleic acid sequence of the fungus-derived HPPD sequence. As used herein, the term “native sequence” refers to the endogenous sequence, i.e., a non-engineered sequence found in the non-engineered plant.

As used herein, the terms “encoding” or “encoded” when used in the context of a specified nucleic acid mean that the nucleic acid comprises the requisite information to direct translation of the nucleotide sequence into a specified protein. The information by which a protein is encoded is specified by the use of codons. A nucleic acid encoding a protein may comprise non-translated sequences (e.g., introns) within translated regions of the nucleic acid or may lack such intervening non-translated sequences (e.g., as in cDNA).

“Variants” is intended to mean substantially similar sequences. For polynucleotides, a variant comprises a deletion and/or addition of one or more nucleotides at one or more internal sites within the reference polynucleotide and/or a substitution of one or more nucleotides at one or more sites in the fungus-derived HPPD polynucleotide. One of skill in the art will recognize that variants of the nucleic acids of the invention will be constructed such that the open reading frame is maintained. For polynucleotides, conservative variants include those sequences that, because of the degeneracy of the genetic code, encode the amino acid sequence of the fungus-derived HPPD polypeptides of the invention. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotide, such as those generated, for example, by using site-directed mutagenesis but which still encode the fungus derived HPPD protein of the invention. Generally, variants of a particular polynucleotide of the invention will have at least about 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Variants of a particular polynucleotide of the invention (i.e., the reference polynucleotide) can also be evaluated by comparison of the percent sequence identity between the polypeptide encoded by a variant polynucleotide and the polypeptide encoded by the reference polynucleotide. Thus, for example, a polynucleotide that encodes a polypeptide with a given percent sequence identity to the polypeptides of SEQ ID NO: 1, 3, 5-8 are disclosed. Percent sequence identity between any two polypeptides can be calculated using sequence alignment programs and parameters described elsewhere herein. Where any given pair of polynucleotides of the invention is evaluated by comparison of the percent sequence identity shared by the two polypeptides they encode, the percent sequence identity between the two encoded polypeptides is at least about 70%, 75%, 80%, 85%, 90%, 91%, 92%, 93%, 94%, 95%, 96%, 97%, 98%, 99% or more sequence identity across the entirety of the HPPD sequences described herein. According to some embodiments, the polypeptide may have exactly the sequence set forth in any of SEQ ID NOs: 1, 3 and 5-8.

As used herein a “variant” protein refers to a protein derived from the reference protein by deletion or addition of one or more amino acids at one or more internal sites in the fungus-derived HPPD protein and/or substitution of one or more amino acids at one or more sites in the fungus-derived HPPD protein. Variant proteins encompassed by the present invention are biologically active, that is, they continue to possess the desired biological activity of the fungus-derived HPPD protein, that is, HPPD enzymatic activity and/or herbicide tolerance as described herein. Such variants may result from, for example, genetic polymorphism or from human manipulation. Biologically active variants will have at least about 70%, 75%, 80%, 85%, 90%, 91%, 92%, 93%, 94%, 95%, 96%, 97%, 98%, 99% or more sequence identity to the fungus-derived HPPD protein of the invention, as determined by sequence alignment programs and parameters described elsewhere herein. A biologically active variant of a protein of the invention may differ from that protein by as few as 1-15 amino acid residues, as few as 1-10, such as 6-10, as few as 5, as few as 4, 3, 2, or even 1 amino acid residue.

The article “a” and “an” are used herein to refer to one or more than one (i.e., to at least one) of the grammatical object of the article. By way of example, “a nucleic acid” means one or more nucleic acids. Throughout the specification the word “comprising,” or variations such as “comprises” will be understood to imply the inclusion of a stated element.

A variety of additional terms are defined or otherwise characterized herein.

### HPPD Sequences

Specifically, the present invention provides HPPD polypeptides that have HPPD enzymatic activity and that confer resistance and/or tolerance in plants to certain classes of herbicides that inhibit HPPD, and variants and fragments thereof. Nucleic acids that encode the native and mutant HPPD polypeptides of the invention are also provided.

The HPPD polypeptides and nucleic acid sequences encoding same are derived from a fungus and may include nucleic acid and optionally amino acid changes at one or more positions relative to the sequence from which they are derived and exhibit enhanced tolerance to one or more HPPD inhibitor herbicides. HPPD enzymes that exhibit enhanced tolerance to an HPPD herbicide may do so by virtue of exhibiting, relative to the native enzyme of the plant.

DNA sequences encoding the fungus derived HPPDs are used in the provision of HPPD plants, crops, plant cells and seeds of the current invention that offer enhanced tolerance or resistance to one or more HPPD herbicides as compared to like plants likewise expressing the unmutated starting enzyme. According to some embodiments, the nucleic acid encoding the fungus derived HPPD may be extracted and isolated from the fungus. According to some embodiments, the nucleic acid encoding the fungus derived HPPD may be synthetically generated.

According to some embodiments, the HPPD, and sequence encoding same, is derived from a fungus of the species Trichoderma. According to some embodiments, the HPPD, and sequence encoding same, is derived from a fungus of the species Trichoderma. According to some embodiments, the HPPD, and sequence encoding same, is derived from a fungus of the species T. asperellum, T. harzianum, T. viride, T. hamatum or any combination thereof. According to some embodiments, the HPPD, and sequence encoding same, is derived from a fungus of the species T. harzianum. According to some embodiments, the HPPD, is derived from the Trichoderma Ti-123 disclosed in U.S. 63/018,027.

According to some embodiments, the HPPD protein has the amino acid sequence set forth in SEQ ID NO: 1, namely:

MSPSAISNSPEQRPANNNGTTPDNFAIQPPADFTGYDHVTWWVGNAKQAA
AYYTTLFGFETTAYRGLETGSRYFASYVVCNNGVRFVFTSPLRSEAHLPE
DETISDSERKLLKEIHAHLERHGDAVKDVAFEVDNVEAVYNKAVAEGAIA
VQGPTATKDDHGSVTTAVICTYGDTTHTLINRRGYTGPFLPGFRAGKERT
SSVEMPNVPLARIDHCVGNQSWNEMVSACAFYEQCLSFHRFWSVDDSQIC
TEFSALNSIVMASPNNLVKMPINEPAPGKKKSQIEEYVIFNSGPGVQHIA
LLTPDIITSVSALRARGVEFINVPTTYYDTMRQRLKTEKRNWQLKEDLDT
IQRLNILIDYDEAGYLLQLFTKPLMDRPTVFIEIIQRNNFEGFGAGNFKS
LFEAIEREQAERGNL

According to some embodiments, the HPPD protein is encoded by the nucleic acid sequence set forth in SEQ ID NO: 2, namely:

ATGTCCCCGTCTGCTATCAGCAACTCCCCAGAGCAGCGACCTGCAAACAA
CAACGGCACCACCCCCGACAACTTCGCTATCCAGCCTCCCGCCGACTTCA
CCGGCTATGACCACGTAACGTGGTGGGTTGGCAACGCCAAGCAGGCGGCC
GCTTATTACACCACCCTCTTTGGGTTCGAGACTACGGCCTATCGTGGACT
CGAGACTGGAAGCCGATACTTCGCTTCCTATGTCGTCTGCAACAATGGCG
TCCGCTTCGTCTTCACGTCGCCTCTGCGATCGGAGGCTCACCTCCCTGAA
GATGAGACCATCTCTGATTCTGAGCGGAAGCTCCTGAAGGAGATTCACGC
TCACCTCGAGAGACACGGCGATGCCGTCAAGGACGTTGCCTTTGAAGTTG
ACAACGTCGAGGCCGTATACAACAAGGCCGTGGCTGAGGGCGCCATCGCC
GTCCAAGGCCCAACCGCCACCAAGGATGATCACGGCTCCGTCACCACGGC
CGTCATCTGCACCTATGGCGATACCACCCACACTCTCATCAACCGCCGGG
GCTACACGGGACCTTTCCTGCCCGGCTTCCGCGCCGGCAAGGAGCGCACC
TCGTCCGTGGAGATGCCCAACGTGCCCCTTGCCCGCATCGACCACTGCGT
CGGCAACCAGTCGTGGAACGAAATGGTCTCGGCCTGCGCCTTTTACGAGC
AGTGCCTGTCCTTCCACCGTTTCTGGTCCGTCGACGACTCCCAGATCTGC
ACCGAGTTCTCGGCCCTCAACTCCATCGTCATGGCCTCGCCCAACAACCT
CGTCAAGATGCCCATCAACGAGCCCGCCCCGGGCAAGAAGAAGTCCCAGA
TCGAGGAGTACGTCATCTTCAACTCCGGCCCGGGCGTCCAGCACATCGCC
CTCCTCACCCCGGACATCATCACCTCCGTCTCGGCCCTCCGCGCCCGCGG
CGTCGAGTTCATCAACGTGCCCACCACTTACTACGACACCATGCGCCAGC
GCCTCAAGACGGAGAAGCGCAACTGGCAGCTCAAGGAGGACCTGGACACC
ATCCAGCGCCTCAACATCCTCATCGACTACGACGAGGCCGGCTACCTCCT
GCAGCTCTTCACCAAGCCGCTCATGGACCGCCCTACCGTCTTCATTGAGA
TTATCCAGAGAAACAACTTTGAGGGCTTCGGCGCCGGCAACTTCAAGAGC
TTGTTCGAGGCCATTGAGCGCGAGCAGGCCGAGCGAGGAAACCTGTAA

According to some embodiments, the HPPD, and sequence encoding same, is derived from a fungus of the species Talaromyces.

According to some embodiments, the HPPD protein has the amino acid sequence set forth in SEQ ID NO: 3, namely:

MAPSAISDLQSDNLPTTQSALSSYRGYDHVHWYVGNAKQAATFYITRMGF
SRVAYRGLETGSRSVCSHVVRNGGITFVLTSPLRSPYNTEKLERLLPSAE
EREYLKEIHEHLARHGDAVKDVAFEVDSVDDVFAAAVQNGAVAVSQPKTV
EDENGQVRVATIRTYGDTTHTLIQRRGVEKPYSGVFLPGYRDETTSGSSD
PITAFLPKVDLRRIDHCVGNQDWDEMEKVCAYYEKVLGFHRFRSVDDKDI
CTDYSALKSIVMSSPNDIVKMPINEPAHGKKQSQIEEYVDFYDGAGVQHI
ALLTDDIISAITNLKARGVEFIKVPPTYYDNMWMRLKKAGMMPKEAWEDI
KKLDILIDFDEGGYLLQLFTKHLMDRPTVFIEIIQRNNFSGFGAGNFKSL
FEAIEREQALRGNLI

According to some embodiments, the HPPD protein is encoded by the nucleic acid sequence set forth in SEQ ID NO: 4, namely:

ATGGCACCATCAGCCATCTCAGACCTCCAATCCGACAACCTACCCACAAC
CCAATCCGCCCTCTCCTCCTACCGCGGCTACGACCATGTACACTGGTACG
TCGGCAACGCCAAACAGGCCGCAACCTTCTACATAACGCGCATGGGATTT
TCTCGTGTCGCCTACCGCGGTCTCGAAACCGGCTCTCGCAGCGTCTGCTC
ACACGTCGTGCGCAACGGCGGTATAACTTTTGTCCTGACCTCGCCGCTTC
GATCACCCTACAACACTGAGAAACTCGAGCGCCTACTTCCCAGTGCTGAA
GAGCGGGAGTATTTGAAAGAGATTCATGAGCATTTGGCACGACATGGTGA
TGCAGTCAAAGACGTCGCGTTTGAGGTCGATTCCGTCGATGATGTGTTCG
CTGCTGCGGTGCAGAATGGCGCCGTTGCGGTCTCGCAACCCAAGACCGTG
GAGGATGAGAATGGTCAAGTGAGGGTTGCCACGATTCGGACGTATGGGGA
TACGACGCATACTTTGATTCAGCGACGGGGGGTCGAAAAGCCGTATTCGG
GCGTTTTCTTGCCAGGGTACAGGGATGAGACGACTTCTGGTAGCAGTGAT
CCTATCACGGCGTTCCTGCCCAAGGTTGATTTGAGGAGGATTGATCATTG
TGTGGGGAATCAGGATTGGGATGAAATGGAGAAGGTCTGCGCGTACTACG
AAAAAGTCCTCGGATTCCACCGTTTCCGGTCCGTAGACGACAAAGACATC
TGCACAGACTACTCCGCCCTGAAATCAATCGTCATGTCCTCGCCCAACGA
CATTGTCAAAATGCCCATCAACGAACCCGCCCACGGCAAAAAACAATCCC
AAATCGAAGAATACGTCGACTTTTACGACGGCGCCGGCGTCCAACACATT
GCCCTGCTGACAGACGACATAATCAGCGCGATCACGAATCTCAAAGCGCG
CGGGGTGGAGTTTATCAAAGTGCCGCCTACGTATTACGATAACATGTGGA
TGCGGCTGAAGAAAGCGGGCATGATGCCCAAGGAGGCGTGGGAGGATATT
AAGAAGTTGGATATTCTGATCGATTTTGATGAGGGAGGGTATTTGTTGCA
GCTCTTCACAAAGCATCTCATGGATCGGCCGACTGTTTTCATTGAGATTA
TTCAGCGCAATAACTTCTCAGGCTTTGGTGCTGGTAATTTCAAGTCGCTG
TTCGAAGCTATTGAACGTGAGCAGGCTCTTAGAGGAAACCTGATCTGA

According to some embodiments, the HPPD, expressed in the plant, is a partial HPPD devoid of 10-50 amino acids at N′ terminus (e.g. devoid of the first 22 or first 38 amino acids).

According to some embodiments, the HPPD, expressed in the plant, is a partial HPPD e.g. SEQ ID NO: 1 devoid of amino acids 1-22 (422). According to some embodiments, the partial A22-HPPD comprises the amino acid sequence set forth in SEQ ID NO: 5

(DNFAIQPPADFTGYDHVTWWVGNAKQAAAYYTTLFGFETTAYRGLET
GSRYFASYVVCNNGVRFVFTSPLRSEAHLPEDETISDSERKLLKEIHA
HLERHGDAVKDVAFEVDNVEAVYNKAVAEGAIAVQGPTATKDDHGSVT
TAVICTYGDTTHTLINRRGYTGPFLPGFRAGKERTSSVEMPNVPLARI
DHCVGNQSWNEMVSACAFYEQCLSFHRFWSVDDSQICTEFSALNSIVM
ASPNNLVKMPINEPAPGKKKSQIEEYVIFNSGPGVQHIALLTPDIITS
VSALRARGVEFINVPTTYYDTMRQRLKTEKRNWQLKEDLDTIQRLNIL
IDYDEAGYLLQLFTKPLMDRPTVFIEIIQRNNFEGFGAGNFKSLFEAI
EREQAERGNL)

According to some embodiments, the HPPD, expressed in the plant, is a partial HPPD e.g. SEQ ID NO: 1 devoid of amino acids 1-38 (438). According to some embodiments, the partial A38-HPPD comprises the amino acid sequence set forth in SEQ ID NO: 6

(VTWWVGNAKQAAAYYTTLFGFETTAYRGLETGSRYFASYVVCNNGVR
FVFTSPLRSEAHLPEDETISDSERKLLKEIHAHLERHGDAVKDVAFEV
DNVEAVYNKAVAEGAIAVQGPTATKDDHGSVTTAVICTYGDTTHTLIN
RRGYTGPFLPGFRAGKERTSSVEMPNVPLARIDHCVGNQSWNEMVSAC
AFYEQCLSFHRFWSVDDSQICTEFSALNSIVMASPNNLVKMPINEPAP
GKKKSQIEEYVIFNSGPGVQHIALLTPDIITSVSALRARGVEFINVPT
TYYDTMRQRLKTEKRNWQLKEDLDTIQRLNILIDYDEAGYLLQLFTKP
LMDRPTVFIEIIQRNNFEGFGAGNFKSLFEAIEREQAERGNL).

According to some embodiments, the HPPD, expressed in the plant, is a partial HPPD e.g. SEQ ID NO: 3 devoid of amino acids 1-22 (422). According to some embodiments, the partial 422-HPPD comprises the amino acid sequence set forth in SEQ ID NO: 7

(PTTQSALSSYRGYDHVHWYVGNAKQAATFYITRMGFSRVAYRGLETG
SRSVCSHVVRNGGITFVLTSPLRSPYNTEKLERLLPSAEEREYLKEIH
EHLARHGDAVKDVAFEVDSVDDVFAAAVQNGAVAVSQPKTVEDENGQV
RVATIRTYGDTTHTLIQRRGVEKPYSGVFLPGYRDETTSGSSDPITAF
LPKVDLRRIDHCVGNQDWDEMEKVCAYYEKVLGFHRERSVDDKDICTD
YSALKSIVMSSPNDIVKMPINEPAHGKKQSQIEEYVDFYDGAGVQHIA
LLTDDIISAITNLKARGVEFIKVPPTYYDNMWMRLKKAGMMPKEAWED
IKKLDILIDFDEGGYLLQLFTKHLMDRPTVFIEIIQRNNFSGFGAGNF
KSLFEAIEREQALRGNLI).

According to some embodiments, the HPPD, expressed in the plant, is a partial HPPD e.g. SEQ ID NO: 3 devoid of amino acids 1-30 (A30). According to some embodiments, the partial A30-HPPD comprises the amino acid sequence set forth in SEQ ID NO: 8

(VHWYVGNAKQAATFYITRMGFSRVAYRGLETGSRSVCSHVVRNGGIT
FVLTSPLRSPYNTEKLERLLPSAEEREYLKEIHEHLARHGDAVKDVAF
EVDSVDDVFAAAVQNGAVAVSQPKTVEDENGQVRVATIRTYGDTTHTL
IQRRGVEKPYSGVFLPGYRDETTSGSSDPITAFLPKVDLRRIDHCVGN
QDWDEMEKVCAYYEKVLGFHRFRSVDDKDICTDYSALKSIVMSSPNDI
VKMPINEPAHGKKQSQIEEYVDFYDGAGVQHIALLTDDIISAITNLKA
RGVEFIKVPPTYYDNMWMRLKKAGMMPKEAWEDIKKLDILIDFDEGGY
LLQLFTKHLMDRPTVFIEIIQRNNFSGFGAGNFKSLFEAIEREQALRG
NLI).

According to some embodiments, the nucleic acid sequence encoding the Trichoderma derived HPPD may have less than 80%, less than 70%, less than 60% or less than 50% similarity to that of the nucleic acid sequences encoding the HPPD enzyme endogenous to the plant in which it is expressed.

According to some embodiments, the nucleic acid sequence and amino acid sequence of the fungus-derived HPPD has less than 50%, less than 40% or less than 30% sequence identity to the HPPD nucleic acid and amino acid sequences of the native plants in which it is expressed.

According to some embodiments, the HPPD enzyme encoded by the fungus-derived HPPD gene exhibits enzymatic activity in plants.

In some embodiment, the polypeptide comprises one or more amino acid substitutions, additions, or deletions. In some embodiment, the polypeptide comprises one or more substitutions corresponding to a conservative variant of the amino acid.

Accordingly, the present invention provides nucleic acid molecules comprising polynucleotide sequences that encode fungus-derived HPPD polypeptides that have HPPD enzymatic activity and that confer resistance or tolerance in plants to certain classes of herbicides that inhibit HPPD, and variants and fragments thereof. In general, the invention includes any polynucleotide sequence that encodes the HPPD polypeptides described herein, as well as any polynucleotide sequence that encodes HPPD polypeptides having one or more conservative amino acid substitutions relative to the HPPD polypeptides described herein. Conservative substitution tables providing functionally similar amino acids are well known in the art. The following five groups each contain amino acids that are conservative substitutions for one another: Nonpolar, aliphatic: Glycine (G), Alanine (A), Valine (V), Leucine (L), Isoleucine (I), Methionine (M); Nonpolar, aromatic: Phenylalanine (F), Tyrosine (Y), Tryptophan (W); Polar, uncharged: Cysteine (C), Serine (S), Threonine (T), Proline (P), Asparagine (N), Glutamine (Q); Basic: Arginine (R), Lysine (K), Histidine (H); Acidic: Aspartic acid (D), Glutamic acid (E).

In one embodiment, the present invention provides a polynucleotide sequence encoding an amino acid sequence having at least about 70%, 75%, 80%, 85%, 90%, 91%, 92%, 93%, 94%, 95%, 96%, 97%, 98%, 99% or more sequence identity to SEQ ID NO: 1, SEQ ID NO: 3, SEQ ID NO: 5, SEQ ID NO: 6, SEQ ID NO: 7 or SEQ ID NO: 8, where the HPPD amino acid sequence is derived from a fungus, where the polypeptide has HPPD enzymatic activity, and where the polypeptide contains one or more substitutions, additions or deletions as discussed. According to some embodiments, the HPPD, and sequence encoding same, is derived from a fungus of the species Trichoderma. According to some embodiments, the HPPD, and sequence encoding same, is derived from a fungus of the species Trichoderma. According to some embodiments, the HPPD, and sequence encoding same, is derived from a fungus of the species T. asperellum, T. harzianum, T. viride, T. hamatum, Trichoderma Ti-123 disclosed in U.S. 63/018,027, or any combination thereof. According to some embodiments, the HPPD, and sequence encoding same, is derived from a fungus of the species Talaromyces. According to some embodiments, the HPPD, and sequence encoding same, is derived from a fungus of the species T. hamatum.

### Gene Stacking

In certain embodiments the polynucleotides of the invention encoding the fungus-derived HPPD polypeptides or variants thereof that retain HPPD enzymatic activity (e.g., a polynucleotide sequence encoding an amino acid sequence selected from the group consisting of SEQ ID NO: 1, 3, 5-8) can be stacked with any combination of polynucleotide sequences of interest in order to create plants with a desired trait. A trait, as used herein, refers to the phenotype derived from a particular sequence or groups of sequences. For example, the polynucleotides encoding the fungus derived HPPD polypeptide or variant thereof that retains HPPD enzymatic activity may be stacked with any other polynucleotides encoding polypeptides that confer a desirable trait, including, but not limited to, resistance to diseases, insects, and herbicides, tolerance to heat and drought, reduced time to crop maturity, improved industrial processing, such as for the conversion of starch or biomass to fermentable sugars, and improved agronomic quality, such as high oil content and high protein content.

In a particular embodiment of the invention, polynucleotides may be stacked (or, alternatively, expression cassettes may be stacked on a single polynucleotide) so as to express more than one type of HPPD polypeptide within a plant. This is a particular advantage where, for example, one HPPD is particularly suitable for providing resistance to one class of HPPD herbicide while the other provides better tolerance to a different class of HPPD herbicide. Stacking HPPD polypeptides is also an advantage where one polypeptide expresses inherent herbicide-resistance but is somewhat labile. This herbicide-resistant HPPD can then be stabilized in mixed expression with, for example, similar but less temperature-labile HPPDs through the formation of mixed enzyme dimers.

Exemplary polynucleotides that may be stacked with polynucleotides of the invention encoding the fungus-derived HPPD polypeptide or variant thereof that retains HPPD enzymatic activity include polynucleotides encoding polypeptides conferring resistance to pests/pathogens such as viruses, nematodes, insects or fungi, and the like. Exemplary polynucleotides that may be stacked with polynucleotides of the invention include polynucleotides encoding: polypeptides having pesticidal and/or insecticidal activity, such as other Bacillus thuringiensis toxic proteins, lectins, pentin, and the like; traits desirable for disease or herbicide resistance (e.g., fumonisin detoxification genes; avirulence and disease resistance genes); a gene encoding an aryloxyalkanoate dioxygenase conferring resistance to certain classes of auxin and acetylCOA carboxylase herbicides or a tfdA gene giving resistance to 2,4 D; a gene encoding a dicamba monoxygenase conferring resistance to dicamba; a gene encoding a homogentisate solanesyltransferase (HST) conferring resistance to HST-inhibiting herbicides; a gene encoding a nitrilase conferring resistance to a nitrile-containing herbicide (e.g. the bxnA bromoxynil nitrilase); acetolactate synthase (ALS) mutants that lead to herbicide resistance such as the S4 and/or Hra mutations; glyphosate resistance (e.g., 5-enol-pyrovyl-shikimate-3-phosphate-synthase (EPSPS) gene, or the glyphosate N-acetyltransferase (GAT) gene); glufosinate resistance (e.g., phosphinothricin acetyl transferase genes PAT and BAR); a cytochrome P450 or variant thereof that confers herbicide resistance or tolerance to, inter alia, HPPD herbicides; and traits desirable for processing or process products such as high oil; modified oils (e.g., fatty acid desaturase genes); modified starches (e.g., ADPG pyrophosphorylases (AGPase), starch synthases (SS), starch branching enzymes (SBE), and starch debranching enzymes (SDBE)); and polymers or bioplastics; beta-ketothiolase, polyhydroxybutyrate synthase, and acetoacetyl-CoA reductase.

According to some embodiments, the desirable trait is resistance or tolerance to glyphosate. In another embodiment, the desirable trait is resistance or tolerance to glufosinate, an HST inhibitor herbicide, an auxin herbicide or a PSII herbicide or any combination thereof.

These stacked combinations can be created by any method including, but not limited to, cross-breeding plants by any conventional or TopCross methodology, or genetic transformation. If the sequences are stacked by genetically transforming the plants, the polynucleotide sequences of interest can be combined at any time and in any order. For example, a transgenic plant comprising one or more desired traits can be used as the target to introduce further traits by subsequent transformation/gene editing. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system.

According to some embodiments, the herein disclosed modified HPPD protein may be translationally fused to a signal peptide. According to some embodiments, the herein disclosed modified HPPD protein may be translationally fused to a chloroplast-targeting peptide (CTP). According to some embodiments, the signal peptide (CTP) may be fused to the N-terminus of the herein disclosed modified HPPD protein or replace the N-terminus of the herein disclosed modified HPPD protein (i.e. be fused to any one of the amino acid sequences set forth in SEQ ID NO: 1, 3 or 5-8). According to some embodiments, the signal peptide may have at least 85%, at least 90% or at least 95% sequence similarity to the CTP consensus sequence set forth in SEQ ID NO: 103, namely: MPPTPTTAAATGAGAAAVTPEHAAFRLV. According to some embodiments, the CTP comprises SEQ ID NO: 103. According to some embodiments, the CTP consists of SEQ ID NO: 103. According to some embodiments, the signal peptide may have at least 85%, at least 90% or at least 95% sequence similarity to the CTP consensus sequence set forth in SEQ ID NO: 104, namely: MGHQNAAVSENQNHDDGAASSP. According to some embodiments, the CTP comprises SEQ ID NO: 104. According to some embodiments, the CTP consists of SEQ ID NO: 104. According to some embodiments, the signal peptide may have at least 85%, at least 90% or at least 95% sequence similarity to the CTP consensus sequence of Arabidopsis thaliana (amino acids 1-38), as set forth in SEQ ID NO: 105, namely: MGHQNAAVSENQNHDDGAASSPGFKLVGFSKFVRKNPKSDKFKVKRFHH. According to some embodiments, the CTP comprises SEQ ID NO: 105. According to some embodiments, the CTP consists of SEQ ID NO: 105.

According to some embodiments, the HPPD, expressed in the plant, is a partial HPPD e.g. SEQ ID NO: 1 devoid of amino acids 1-22 (422) and comprising amino acids 1-22 of Arabidopsis thaliana as set forth in SEQ ID NO: 106

(MGHQNAAVSENQNHDDGAASSPDNFAIQPPADFTGYDHVTWWVGNAK
QAAAYYTTLFGFETTAYRGLETGSRYFASYVVCNNGVRFVFTSPLRSE
AHLPEDETISDSERKLLKEIHAHLERHGDAVKDVAFEVDNVEAVYNKA
VAEGAIAVQGPTATKDDHGSVTTAVICTYGDTTHTLINRRGYTGPFLP
GFRAGKERTSSVEMPNVPLARIDHCVGNQSWNEMVSACAFYEQCLSFH
RFWSVDDSQICTEFSALNSIVMASPNNLVKMPINEPAPGKKKSQIEEY
VIFNSGPGVQHIALLTPDIITSVSALRARGVEFINVPTTYYDTMRQRL
KTEKRNWQLKEDLDTIQRLNILIDYDEAGYLLQLFTKPLMDRPTVFIE
IIQRNNFEGFGAGNFKSLFEAIEREQAERGNL)

According to some embodiments, the HPPD, expressed in the plant, is a partial HPPD e.g. SEQ ID NO: 1 devoid of amino acids 1-22 (A22) and comprising amino acids 1-49 of Arabidopsis thaliana as set forth in SEQ ID NO: 107

(MGHQNAAVSENQNHDDGAASSPGFKLVGFSKFVRKNPKSDKFKVKRF
HHDNFAIQPPADFTGYDHVTWWVGNAKQAAAYYTTLFGFETTAYRGLE
TGSRYFASYVVCNNGVRFVFTSPLRSEAHLPEDETISDSERKLLKEIH
AHLERHGDAVKDVAFEVDNVEAVYNKAVAEGAIAVQGPTATKDDHGSV
TTAVICTYGDTTHTLINRRGYTGPFLPGFRAGKERTSSVEMPNVPLAR
IDHCVGNQSWNEMVSACAFYEQCLSFHRFWSVDDSQICTEFSALNSIV
MASPNNLVKMPINEPAPGKKKSQIEEYVIFNSGPGVQHIALLTPDIIT
SVSALRARGVEFINVPTTYYDTMRQRLKTEKRNWQLKEDLDTIQRLNI
LIDYDEAGYLLQLFTKPLMDRPTVFIEIIQRNNFEGFGAGNFKSLFEA
IEREQAERGNL)

According to some embodiments, the HPPD, expressed in the plant, is a partial HPPD e.g. SEQ ID NO: 1 devoid of amino acids 1-38 (438) and comprising amino acids 1-22 of Arabidopsis thaliana as set forth in SEQ ID NO: 108

(MGHQNAAVSENQNHDDGAASSPVTWWVGNAKQAAAYYTTLFGFETTA
YRGLETGSRYFASYVVCNNGVRFVFTSPLRSEAHLPEDETISDSERKL
LKEIHAHLERHGDAVKDVAFEVDNVEAVYNKAVAEGAIAVQGPTATKD
DHGSVTTAVICTYGDTTHTLINRRGYTGPFLPGFRAGKERTSSVEMPN
VPLARIDHCVGNQSWNEMVSACAFYEQCLSFHRFWSVDDSQICTEFSA
LNSIVMASPNNLVKMPINEPAPGKKKSQIEEYVIFNSGPGVQHIALLT
PDIITSVSALRARGVEFINVPTTYYDTMRQRLKTEKRNWQLKEDLDTI
QRLNILIDYDEAGYLLQLFTKPLMDRPTVFIEIIQRNNFEGFGAGNFK
SLFEAIEREQAERGNL).

According to some embodiments, the HPPD, expressed in the plant, is a partial HPPD e.g. SEQ ID NO: 1 devoid of amino acids 1-38 (438) and comprising amino acids 1-49 of Arabidopsis thaliana as set forth in SEQ ID NO: 109

(MGHQNAAVSENQNHDDGAASSPGFKLVGFSKFVRKNPKSDKFKVKRF
HHVTWWVGNAKQAAAYYTTLFGFETTAYRGLETGSRYFASYVVCNNGV
RFVFTSPLRSEAHLPEDETISDSERKLLKEIHAHLERHGDAVKDVAFE
VDNVEAVYNKAVAEGAIAVQGPTATKDDHGSVTTAVICTYGDTTHTLI
NRRGYTGPFLPGFRAGKERTSSVEMPNVPLARIDHCVGNQSWNEMVSA
CAFYEQCLSFHRFWSVDDSQICTEFSALNSIVMASPNNLVKMPINEPA
PGKKKSQIEEYVIFNSGPGVQHIALLTPDIITSVSALRARGVEFINVP
TTYYDTMRQRLKTEKRNWQLKEDLDTIQRLNILIDYDEAGYLLQLFTK
PLMDRPTVFIEIIQRNNFEGFGAGNEKSLFEAIEREQAERGNL).

According to some embodiments, the HPPD, expressed in the plant, is a partial HPPD e.g. SEQ ID NO: 3 devoid of amino acids 1-22 (A22) and comprising amino acids 1-22 of Arabidopsis thaliana as set forth in SEQ ID NO: 110

(MGHQNAAVSENQNHDDGAASSPPTTQSALSSYRGYDHVHWYVGNAKQ
AATFYITRMGFSRVAYRGLETGSRSVCSHVVRNGGITFVLTSPLRSPY
NTEKLERLLPSAEEREYLKEIHEHLARHGDAVKDVAFEVDSVDDVFAA
AVQNGAVAVSQPKTVEDENGQVRVATIRTYGDTTHTLIQRRGVEKPYS
GVFLPGYRDETTSGSSDPITAFLPKVDLRRIDHCVGNQDWDEMEKVCA
YYEKVLGFHRFRSVDDKDICTDYSALKSIVMSSPNDIVKMPINEPAHG
KKQSQIEEYVDFYDGAGVQHIALLTDDIISAITNLKARGVEFIKVPPT
YYDNMWMRLKKAGMMPKEAWEDIKKLDILIDFDEGGYLLQLFTKHLMD
RPTVFIEIIQRNNFSGFGAGNFKSLFEAIEREQALRGNLI).

According to some embodiments, the HPPD, expressed in the plant, is a partial HPPD e.g. SEQ ID NO: 3 devoid of amino acids 1-22 (422) and comprising amino acids 1-49 of Arabidopsis thaliana as set forth in SEQ ID NO: 111

(MGHQNAAVSENQNHDDGAASSPGFKLVGFSKFVRKNPKSDKFKVKRF
HHPTTQSALSSYRGYDHVHWYVGNAKQAATFYITRMGFSRVAYRGLET
GSRSVCSHVVRNGGITFVLTSPLRSPYNTEKLERLLPSAEEREYLKEI
HEHLARHGDAVKDVAFEVDSVDDVFAAAVQNGAVAVSQPKTVEDENGQ
VRVATIRTYGDTTHTLIQRRGVEKPYSGVFLPGYRDETTSGSSDPITA
FLPKVDLRRIDHCVGNQDWDEMEKVCAYYEKVLGFHRFRSVDDKDICT
DYSALKSIVMSSPNDIVKMPINEPAHGKKQSQIEEYVDFYDGAGVQHI
ALLTDDIISAITNLKARGVEFIKVPPTYYDNMWMRLKKAGMMPKEAWE
DIKKLDILIDFDEGGYLLQLFTKHLMDRPTVFIEIIQRNNFSGFGAGN
FKSLFEAIEREQALRGNLI).

According to some embodiments, the HPPD, expressed in the plant, is a partial HPPD e.g. SEQ ID NO: 3 devoid of amino acids 1-30 (A30) and comprising amino acids 1-22 of Arabidopsis thaliana as set forth in SEQ ID NO: 112

(MGHQNAAVSENQNHDDGAASSPVHWYVGNAKQAATFYITRMGFSRVAYR
GLETGSRSVCSHVVRNGGITFVLTSPLRSPYNTEKLERLLPSAEEREYLK
EIHEHLARHGDAVKDVAFEVDSVDDVFAAAVQNGAVAVSQPKTVEDENGQ
VRVATIRTYGDTTHTLIQRRGVEKPYSGVFLPGYRDETTSGSSDPITAFL
PKVDLRRIDHCVGNQDWDEMEKVCAYYEKVLGFHRFRSVDDKDICTDYSA
LKSIVMSSPNDIVKMPINEPAHGKKQSQIEEYVDFYDGAGVQHIALLTDD
IISAITNLKARGVEFIKVPPTYYDNMWMRLKKAGMMPKEAWEDIKKLDIL
IDFDEGGYLLQLFTKHLMDRPTVFIEIIQRNNFSGFGAGNFKSLFEAIER
EQALRGNLI).

According to some embodiments, the HPPD, expressed in the plant, is a partial HPPD e.g. SEQ ID NO: 3 devoid of amino acids 1-30 (A30) and comprising amino acids 1-49 of Arabidopsis thaliana as set forth in SEQ ID NO: 113

(MGHQNAAVSENQNHDDGAASSPGFKLVGFSKFVRKNPKSDKFKVKRF
HHVHWYVGNAKQAATFYITRMGFSRVAYRGLETGSRSVCSHVVRNGGI
TFVLTSPLRSPYNTEKLERLLPSAEEREYLKEIHEHLARHGDAVKDVA
FEVDSVDDVFAAAVQNGAVAVSQPKTVEDENGQVRVATIRTYGDTTHT
LIQRRGVEKPYSGVFLPGYRDETTSGSSDPITAFLPKVDLRRIDHCVG
NQDWDEMEKVCAYYEKVLGFHRFRSVDDKDICTDYSALKSIVMSSPND
IVKMPINEPAHGKKQSQIEEYVDFYDGAGVQHIALLTDDIISAITNLK
ARGVEFIKVPPTYYDNMWMRLKKAGMMPKEAWEDIKKLDILIDFDEGG
YLLQLFTKHLMDRPTVFIEIIQRNNFSGFGAGNFKSLFEAIEREQALR
GNLI)

According to some embodiments, the HPPD, expressed in the plant, is wt HPPD e.g. SEQ ID NO: 1 to which amino acids 1-22 of Arabidopsis thaliana has been added as set forth in SEQ ID NO: 114

(MGHQNAAVSENQNHDDGAASSPMSPSAISNSPEQRPANNNGTTPDNF
AIQPPADFTGYDHVTWWVGNAKQAAAYYTTLFGFETTAYRGLETGSRY
FASYVVCNNGVRFVFTSPLRSEAHLPEDETISDSERKLLKEIHAHLER
HGDAVKDVAFEVDNVEAVYNKAVAEGAIAVQGPTATKDDHGSVTTAVI
CTYGDTTHTLINRRGYTGPFLPGFRAGKERTSSVEMPNVPLARIDHCV
GNQSWNEMVSACAFYEQCLSFHRFWSVDDSQICTEFSALNSIVMASPN
NLVKMPINEPAPGKKKSQIEEYVIFNSGPGVQHIALLTPDIITSVSAL
RARGVEFINVPTTYYDTMRQRLKTEKRNWQLKEDLDTIQRLNILIDYD
EAGYLLQLFTKPLMDRPTVFIEIIQRNNFEGFGAGNFKSLFEAIEREQ
AERGNL)

According to some embodiments, the HPPD, expressed in the plant, is wt HPPD e.g. SEQ ID NO: 1 to which amino acids 1-49 of Arabidopsis thaliana has been added as set forth in SEQ ID NO: 115

(MGHQNAAVSENQNHDDGAASSPGFKLVGFSKFVRKNPKSDKFKVKRF
HHMSPSAISNSPEQRPANNNGTTPDNFAIQPPADFTGYDHVTWWVGNA
KQAAAYYTTLFGFETTAYRGLETGSRYFASYVVCNNGVRFVFTSPLRS
EAHLPEDETISDSERKLLKEIHAHLERHGDAVKDVAFEVDNVEAVYNK
AVAEGAIAVQGPTATKDDHGSVTTAVICTYGDTTHTLINRRGYTGPFL
PGFRAGKERTSSVEMPNVPLARIDHCVGNQSWNEMVSACAFYEQCLSF
HRFWSVDDSQICTEFSALNSIVMASPNNLVKMPINEPAPGKKKSQIEE
YVIFNSGPGVQHIALLTPDIITSVSALRARGVEFINVPTTYYDTMRQR
LKTEKRNWQLKEDLDTIQRLNILIDYDEAGYLLQLFTKPLMDRPTVFI
EIIQRNNFEGFGAGNFKSLFEAIEREQAERGNL)

According to some embodiments, the HPPD, expressed in the plant, is wt HPPD e.g. SEQ ID NO: 3 to which amino acids 1-22 of Arabidopsis thaliana has been added as set forth in SEQ ID NO: 116

(MGHQNAAVSENQNHDDGAASSPMAPSAISDLQSDNLPTTQSALSSYR
GYDHVHWYVGNAKQAATFYITRMGFSRVAYRGLETGSRSVCSHVVRNG
GITFVLTSPLRSPYNTEKLERLLPSAEEREYLKEIHEHLARHGDAVKD
VAFEVDSVDDVFAAAVQNGAVAVSQPKTVEDENGQVRVATIRTYGDTT
HTLIQRRGVEKPYSGVFLPGYRDETTSGSSDPITAFLPKVDLRRIDHC
VGNQDWDEMEKVCAYYEKVLGFHRFRSVDDKDICTDYSALKSIVMSSP
NDIVKMPINEPAHGKKQSQIEEYVDFYDGAGVQHIALLTDDIISAITN
LKARGVEFIKVPPTYYDNMWMRLKKAGMMPKEAWEDIKKLDILIDFDE
GGYLLQLFTKHLMDRPTVFIEIIQRNNFSGFGAGNFKSLFEAIEREQA
LRGNLI)

According to some embodiments, the HPPD, expressed in the plant, is wt HPPD e.g. SEQ ID NO: 3 to which amino acids 1-49 of Arabidopsis thaliana has been added as set forth in SEQ ID NO: 117

(MGHQNAAVSENQNHDDGAASSPGFKLVGFSKFVRKNPKSDKFKVKRF
HHMAPSAISDLQSDNLPTTQSALSSYRGYDHVHWYVGNAKQAATFYIT
RMGFSRVAYRGLETGSRSVCSHVVRNGGITFVLTSPLRSPYNTEKLER
LLPSAEEREYLKEIHEHLARHGDAVKDVAFEVDSVDDVFAAAVQNGAV
AVSQPKTVEDENGQVRVATIRTYGDTTHTLIQRRGVEKPYSGVFLPGY
RDETTSGSSDPITAFLPKVDLRRIDHCVGNQDWDEMEKVCAYYEKVLG
FHRFRSVDDKDICTDYSALKSIVMSSPNDIVKMPINEPAHGKKQSQIE
EYVDFYDGAGVQHIALLTDDIISAITNLKARGVEFIKVPPTYYDNMWM
RLKKAGMMPKEAWEDIKKLDILIDFDEGGYLLQLFTKHLMDRPTVFIE
IIQRNNFSGFGAGNFKSLFEAIEREQALRGNLI)

### Plant Expression Cassettes

The compositions of the invention may additionally contain nucleic acid sequences for transformation and expression in a plant of interest. The nucleic acid sequences may be present in DNA constructs or expression cassettes. The term “Expression cassette” as used herein means a nucleic acid molecule capable of directing expression of a particular nucleotide sequence in an appropriate host cell, comprising a promoter operatively linked to the nucleotide sequence of interest (i.e., a polynucleotide encoding the herein disclosed fungus-derived HPPD polypeptide or variant thereof that retains HPPD enzymatic activity, alone or in combination with one or more additional nucleic acid molecules encoding polypeptides that confer desirable traits, which is operatively linked to termination signals). It also typically comprises sequences required for proper translation of the nucleotide sequence. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. Typically, however, the expression cassette is heterologous with respect to the host, i.e., the particular DNA sequence of the expression cassette does not occur naturally in the host cell and must have been introduced into the host cell or an ancestor of the host cell by a transformation event. The expression of the nucleotide sequence in the expression cassette may be under the control of a constitutive promoter or of an inducible promoter that initiates transcription only when the host cell is exposed to some particular external stimulus. Additionally, the promoter can also be specific to a particular tissue or organ or stage of development.

The present invention encompasses the transformation of plants with expression cassettes capable of expressing a polynucleotide of interest, i.e., a polynucleotide encoding the fungus-derived HPPD polypeptide or variant thereof that retains HPPD enzymatic activity, alone or in combination with one or more additional nucleic acid molecules encoding polypeptides that confer desirable traits. The expression cassette will include in the 5′-3′ direction of transcription, a transcriptional and translational initiation region (i.e., a promoter) and a polynucleotide open reading frame. The expression cassette may optionally comprise a transcriptional and translational termination region (i.e., termination region) functional in plants. In some embodiments, the expression cassette comprises a selectable marker gene to allow for selection for stable transformants. Expression constructs of the invention may also comprise a leader sequence and/or a sequence allowing for inducible expression of the polynucleotide of interest.

The regulatory sequences of the expression construct are operably linked to the polynucleotide of interest. By “operably linked” is intended a functional linkage between a promoter and a second sequence wherein the promoter sequence initiates and mediates transcription of the DNA sequence corresponding to the second sequence. Generally, operably linked means that the nucleotide sequences being linked are contiguous.

Any promoter capable of driving expression in the plant of interest may be used in the practice of the invention. The promoter may be native or analogous or foreign or heterologous to the plant host. The terms “heterologous” and “exogenous” when used herein to refer to a nucleic acid sequence (e.g. a DNA or RNA sequence) or a gene, refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides.

A “homologous” nucleic acid (e.g., DNA) sequence is a nucleic acid (e.g., DNA or RNA) sequence naturally associated with a host cell into which it is introduced.

The choice of promoters to be included depends upon several factors, including, but not limited to, efficiency, selectability, inducibility, desired expression level, and cell- or tissue-preferential expression. It is a routine matter for one of skill in the art to modulate the expression of a sequence by appropriately selecting and positioning promoters and other regulatory regions relative to that sequence. The promoters that are used for expression of the transgene(s) can be a strong plant promoter, a viral promoter, or a chimeric promoter composed of elements such as: TATA box from any gene (or synthetic, based on analysis of plant gene TATA boxes), optionally fused to the region 5′ to the TATA box of plant promoters (which direct tissue and temporally appropriate gene expression), optionally fused to 1 or more enhancers (such as the 35S enhancer, FMV enhancer, CMP enhancer, RUBISCO SMALL SUBUNIT enhancer, PLASTOCYANIN enhancer).

Exemplary constitutive promoters include, for example, the core promoter of the Rsyn7 promoter and other constitutive promoters; the core CaMV 35S promoter; rice actin; ubiquitin; pEMU; MAS; ALS promoter, and the like.

Appropriate plant or chimeric promoters are useful for applications such as expression of transgenes in certain tissues, while minimizing expression in other tissues, such as seeds, or reproductive tissues. Exemplary cell type- or tissue-preferential promoters drive expression preferentially in the target tissue but may also lead to some expression in other cell types or tissues as well.

In other embodiments of the present invention, inducible promoters may be desired. Inducible promoters drive transcription in response to external stimuli such as chemical agents or environmental stimuli. For example, inducible promoters can confer transcription in response to hormones such as gibberellic acid or ethylene, or in response to light or drought.

A variety of transcriptional terminators are available for use in expression cassettes. These are responsible for the termination of transcription beyond the transgene and correct mRNA polyadenylation. The termination region may be native with the transcriptional initiation region, may be native with the operably linked DNA sequence of interest, may be native with the plant host, or may be derived from another source (i.e., foreign or heterologous to the promoter, the DNA sequence of interest, the plant host, or any combination thereof). Appropriate transcriptional terminators are those that are known to function in plants and include the CAMV 35S terminator, the tml terminator, the nopaline synthase terminator, the octopine synthase terminator and the pea rbcs E9 terminator. These can be used in both monocotyledons and dicotyledons. In addition, a gene's native transcription terminator may be used.

Generally, the expression cassette will comprise a selectable marker gene for the selection of transformed cells. Selectable marker genes are utilized for the selection of transformed cells or tissues.

Numerous sequences have been found to enhance gene expression from within the transcriptional unit and these sequences can be used in conjunction with the genes of this invention to increase their expression in plants.

Various intron sequences have been shown to enhance expression, particularly in monocotyledonous cells. For example, the introns of the maize Adh1 gene have been found to significantly enhance the expression of the wild-type gene under its cognate promoter when introduced into maize cells. Intron 1 was found to be particularly effective and enhanced expression in fusion constructs with the chloramphenicol acetyltransferase gene. In the same experimental system, the intron from the maize bronze 1 gene had a similar effect in enhancing expression. Intron sequences have been routinely incorporated into plant transformation vectors, typically within the non-translated leader.

The present invention also relates to nucleic acid constructs comprising one or more of the expression cassettes described above. The construct can be a vector, such as a plant transformation vector. In one embodiment, the vector is a plant transformation vector comprising a polynucleotide comprising the sequence set forth in SEQ ID NO: 2, 4 or any other polynucleotide encoding the amino acid sequence set forth in SEQ ID NO: 1, 3, and/or 5-8.

### Plants

As used herein, the term “plant part” or “plant tissue” includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, cars, cobs, husks, stalks, roots, root tips, anthers, and the like. The aforementioned term also includes plant products, such as grain, fruits, and nuts.

The plants disclosed herein are genetically modified to express an HPPD enzyme not naturally expressed in the plant (or in plants in general), such as to express the herein disclosed fungus-derived HPPD polypeptide or variant thereof that retains HPPD enzymatic activity in the plant.

As used herein, the term “genetically modified” may refer to transgenic plants transformed to stably express the non-naturally occurring HPPD enzyme as well as to plants being modified genetically to express a non-naturally occurring HPPD enzyme (mutation or substitution) using gene editing technologies, such as but not limited to CRISPR/Cas, TALLEN and zink-finger technologies. According to some embodiments, the genetically modified plant is a transgenic plant. According to some embodiments, the genetically modified plant having a HPPD enzyme modified by gene editing. According to some embodiments, the HPPD enzyme may be an exogenously introduced HPPD enzyme, such as, but not limited to the HPPD enzymes derived from Trichoderma spp or Talaromyces spp disclosed herein. According to some embodiments, the HPPD enzyme may be the endogenous HPPD of the plant genetically modified to include fungal derived motifs and/or mutations, rendering the plant resistant to the HPPD inhibitors.

The type of plant selected depends on a variety of factors, including for example, the downstream use of the harvested plant material, amenability of the plant species to transformation, and the conditions under which the plants will be grown, harvested, and/or processed. One of skill will further recognize that additional factors for selecting appropriate plant varieties for use in the present invention include high yield potential, good stalk strength, resistance to specific diseases, drought tolerance, rapid dry down and grain quality sufficient to allow storage and shipment to market with minimum loss.

Plants according to the present invention include any plant that is cultivated for the purpose of producing plant material that is sought after by man or animal for either oral consumption, or for utilization in an industrial, pharmaceutical, or commercial process. The invention may be applied to any of a variety of plants, including, but not limited to maize, wheat, rice, barley, soybean, cowpea, chickpea, cotton, sorghum, oat, beans in general, rape/canola, alfalfa, flax, sunflower, safflower, millet, rye, sugarcane, sugar beet, cocoa, tea, Brassica, cotton, coffee, sweet potato, flax, peanut, clover; vegetables such as lettuce, tomato, cucurbits, cassava, potato, carrot, radish, pea, lentils, cabbage, cauliflower, broccoli, Brussels sprouts, peppers, and pineapple; tree fruits such as citrus, apples, pears, peaches, apricots, walnuts, avocado, banana, palm, eucalyptus, poplar, pine and coconut; and flowers such as orchids, petunia, carnations and roses. Other plants useful in the practice of the invention include perennial grasses, such as switchgrass, prairie grasses, indiangrass, big bluestem grass and the like. It is recognized that mixtures of plants may be used.

In addition, the term “crop” is to be understood as also including crop that have been rendered tolerant to herbicides or classes of herbicides (such as, for example, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors) as a result of conventional methods of breeding or genetic engineering. Examples of crops that have been rendered tolerant to herbicides or classes of herbicides by genetic engineering methods include glyphosate- and glufosinate-resistant crop varieties. The method according to the present invention is especially suitable for the protection of crops where HPPD herbicides are used for weed control.

### Plant Transformation

Once an herbicide resistant or tolerant HPPD polynucleotide, alone or in combination with one or more additional nucleic acid molecules encoding polypeptides that confer desirable traits, has been cloned into an expression system, it is transformed into a plant cell. The expression cassettes of the present invention can be introduced into the plant cell in a number of art-recognized ways. The term “introducing” in the context of a polynucleotide, for example, a nucleotide construct of interest, is intended to mean presenting to the plant the polynucleotide in such a manner that the polynucleotide gains access to the interior of a cell of the plant. Where more than one polynucleotide is to be introduced, these polynucleotides can be assembled as part of a single nucleotide construct, or as separate nucleotide constructs, and can be located on the same or different transformation vectors. Accordingly, these polynucleotides can be introduced into the host cell of interest in a single transformation event, in separate transformation events, or, for example, in plants, as part of a breeding protocol. The methods of the invention do not depend on a particular method for introducing one or more polynucleotides into a plant, only that the polynucleotide(s) gains access to the interior of at least one cell of the plant. Methods for introducing polynucleotides into plants are known in the art including, but not limited to, transient transformation methods, stable transformation methods, and virus-mediated methods.

“Transient transformation” in the context of a polynucleotide is intended to mean that a polynucleotide is introduced into the plant and does not integrate into the genome of the plant. “Stable transformation” or “stably transformed” is intended to mean that a polynucleotide, for example, a nucleotide construct described herein, introduced into a plant integrates into the genome of the plant and is capable of being inherited by the progeny thereof, more particularly, by the progeny of multiple successive generations.

Numerous transformation vectors available for plant transformation are known to those of ordinary skill in the plant transformation arts, and the genes pertinent to this invention can be used in conjunction with any such vectors. The selection of vector will depend upon the preferred transformation technique and the target species for transformation. For certain target species, different antibiotic or herbicide selection markers may be preferred. According to some embodiments, the HPPD gene of the current invention is, in combination with the use of an HPPD herbicide as selection agent, itself used as the selectable marker.

Methods for regeneration of plants are also well known in the art. For example, Ti plasmid vectors have been utilized for the delivery of foreign DNA, as well as direct DNA uptake, liposomes, electroporation, microinjection, and microprojectiles. In addition, bacteria from the genus Agrobacterium can be utilized to transform plant cells. Below are descriptions of representative techniques for transforming both dicotyledonous and monocotyledonous plants, as well as a representative plastid transformation technique.

Another approach to transforming plant cells with a gene involves propelling inert or biologically active particles at plant tissues and cells. Generally, this procedure involves propelling inert or biologically active particles at the cells under conditions effective to penetrate the outer surface of the cell and afford incorporation within the interior thereof. When inert particles are utilized, the vector can be introduced into the cell by coating the particles with the vector containing the desired gene. Alternatively, the target cell can be surrounded by the vector so that the vector is carried into the cell by the wake of the particle. Biologically active particles (e.g., dried yeast cells, dried bacterium or a bacteriophage, each containing DNA sought to be introduced) can also be propelled into plant cell tissue.

Transformation of most monocotyledon species has now also become routine. Preferred techniques include direct gene transfer into protoplasts using PEG or electroporation techniques, and particle bombardment into callus tissue. Transformations can be undertaken with a single DNA species or multiple DNA species (i.e., co-transformation) and both of these techniques are suitable for use with this invention. Co-transformation may have the advantage of avoiding complete vector construction and of generating transgenic plants with unlinked loci for the gene of interest and the selectable marker, enabling the removal of the selectable marker in subsequent generations, should this be regarded desirable. However, a disadvantage of the use of co-transformation is the less than 100% frequency with which separate DNA species are integrated into the genome.

The plants obtained via transformation with a nucleic acid sequence of interest in the present invention can be any of a wide variety of plant species, including those of monocots and dicots; however, the plants used in the method of the invention are preferably selected from the list of agronomically important target crops set forth elsewhere herein. The expression of a gene of the present invention in combination with other characteristics important for production and quality can be incorporated into plant lines through breeding.

The genetic properties engineered into the seeds and plants described above may be passed on by sexual reproduction or vegetative growth and can thus be maintained and propagated in progeny plants. Generally, maintenance and propagation make use of known agricultural methods developed to fit specific purposes such as tilling, sowing or harvesting.

Use of the advantageous genetic properties of the plants and seeds according to the invention can further be made in plant breeding. Depending on the desired properties, different breeding measures are taken. The relevant techniques are well known in the art and include but are not limited to hybridization, inbreeding, backcross breeding, multi-line breeding, variety blend, interspecific hybridization, aneuploid techniques, etc. Thus, the seeds and plants according to the invention can be used for the breeding of improved plant lines that, for example, increase the effectiveness of conventional methods such as herbicide or pesticide treatment or allow one to dispense with said methods due to their modified genetic properties.

### Herbicide Resistance

The present invention provides plants, plant cells, tissues, and seeds that have been genetically modified with a nucleic acid molecule encoding a fungus derived HPPD or variant thereof and/or to plants in which the endogenous HPPD has been edited (e.g. by CRISPR/Cas technology, TALLEN, Zink-finger etc.) to include fungus derived motifs and/or mutations, thereby providing plants with an HPPD that confers resistance or tolerance to herbicides, optionally combination with one or more additional nucleic acid molecules encoding polypeptides that confer desirable traits.

In one embodiment, the plants of the invention exhibit resistance or tolerance to application of herbicide in an amount of from about 5 to about 2,000 grams per hectare (g/ha), including, for example, about 5 g/ha, about 10 g/ha, about 15 g/ha, about 20 g/ha, about 25 g/ha, about 30 g/ha, about 35 g/ha, about 40 g/ha, about 45 g/ha, about 50 g/ha, about 55 g/ha, about 60 g/ha, about 65 g/ha, about 70 g/ha, about 75 g/ha, about 80 g/ha, about 85 g/ha, about 90 g/ha, about 95 g/ha, about 100 g/ha, about 110 g/ha, about 120 g/ha, about 130 g/ha, about 140 g/ha, about 150 g/ha, about 160 g/ha, about 170 g/ha, about 180 g/ha, about 190 g/ha, about 200 g/ha, about 210 g/ha, about 220 g/ha, about 230 g/ha, about 240 g/ha, about 250 g/ha, about 260 g/ha, about 270 g/ha, about 280 g/ha, about 290 g/ha, about 300 g/ha, about 310 g/ha, about 320 g/ha, about 330 g/ha, about 340 g/ha, about 350 g/ha, about 360 g/ha, about 370 g/ha, about 380 g/ha, about 390 g/ha, about 400 g/ha, about 410 g/ha, about 420 g/ha, about 430 g/ha, about 440 g/ha, about 450 g/ha, about 460 g/ha, about 470 g/ha, about 480 g/ha, about 490 g/ha, about 500 g/ha, about 510 g/ha, about 520 g/ha, about 530 g/ha, about 540 g/ha, about 550 g/ha, about 560 g/ha, about 570 g/ha, about 580 g/ha, about 590 g/ha, about 600 g/ha, about 610 g/ha, about 620 g/ha, about 630 g/ha, about 640 g/ha, about 650 g/ha, about 660 g/ha, about 670 g/ha, about 680 g/ha, about 690 g/ha, about 700 g/ha, about 710 g/ha, about 720 g/ha, about 730 g/ha, about 740 g/ha, about 750 g/ha, about 760 g/ha, about 770 g/ha, about 780 g/ha, about 790 g/ha, about 800 g/ha, about 810 g/ha, about 820 g/ha, about 830 g/ha, about 840 g/ha, about 850 g/ha, about 860 g/ha, about 870 g/ha, about 880 g/ha, about 890 g/ha, about 900 g/ha, about 910 g/ha, about 920 g/ha, about 930 g/ha, about 940 g/ha, about 950 g/ha, about 960 g/ha, about 970 g/ha, about 980 g/ha, about 990 g/ha, about 1,000 g/ha, about 1,010 g/ha, about 1,020 g/ha, about 1,030 g/ha, about 1,040 g/ha, about 1,050 g/ha, about 1,060 g/ha, about 1,070 g/ha, about 1,080 g/ha, about 1,090 g/ha, about 1,100 g/ha, about 1,110 g/ha, about 1,120 g/ha, about 1,130 g/ha, about 1,140 g/ha, about 1,150 g/ha, about 1,160 g/ha, about 1,170 g/ha, about 1,180 g/ha, about 1,190 g/ha, about 1,200 g/ha, about 1,210 g/ha, about 1,220 g/ha, about 1,230 g/ha, about 1,240 g/ha, about 1,250 g/ha, about 1,260 g/ha, about 1,270 g/ha, about 1,280 g/ha, about 1,290 g/ha, about 1,300 g/ha, about 1,310 g/ha, about 1,320 g/ha, about 1,330 g/ha, about 1,340 g/ha, about 1,350 g/ha, about 1,360 g/ha, about 1,370 g/ha, about 1,380 g/ha, about 1,390 g/ha, about 1,400 g/ha, about 1,410 g/ha, about 1,420 g/ha, about 1,430 g/ha, about 1,440 g/ha, about 1,450 g/ha, about 1,460 g/ha, about 1,470 g/ha, about 1,480 g/ha, about 1,490 g/ha, about 1,500 g/ha, about 1,510 g/ha, about 1,520 g/ha, about 1,530 g/ha, about 1,540 g/ha, about 1,550 g/ha, about 1,560 g/ha, about 1,570 g/ha, about 1,580 g/ha, about 1,590 g/ha, about 1,600 g/ha, about 1,610 g/ha, about 1,620 g/ha, about 1,630 g/ha, about 1,640 g/ha, about 1,650 g/ha, about 1,660 g/ha, about 1,670 g/ha, about 1,680 g/ha, about 1,690 g/ha, about 1,700 g/ha, about 1,710 g/ha, about 1,720 g/ha, about 1,730 g/ha, about 1,740 g/ha, about 1,750 g/ha, about 1,760 g/ha, about 1,770 g/ha, about 1,780 g/ha, about 1,790 g/ha, about 1,800 g/ha, about 1,810 g/ha, about 1,820 g/ha, about 1,830 g/ha, about 1,840 g/ha, about 1,850 g/ha, about 1,860 g/ha, about 1,870 g/ha, about 1,880 g/ha, about 1,890 g/ha, about 1,900 g/ha, about 1,910 g/ha, about 1,920 g/ha, about 1,930 g/ha, about 1,940 g/ha, about 1,950 g/ha, about 1,960 g/ha, about 1,970 g/ha, about 1,980 g/ha, about 1,990 g/ha, or about 2,000 g/ha.

The methods of the present invention are especially useful to protect crops from the herbicidal injury of HPPD inhibitor herbicides. For example, the HPPD inhibiting herbicide is suitably selected from the group consisting of bicyclopyrone (CAS RN 352010-68-5), benzobicyclon (CAS RN 156963-66-5), benzofenap (CAS RN 82692-44-2), ketospiradox (CAS RN 192708-91-1) or its free acid (CAS RN 187270-87-7), isoxachlortole (CAS RN 141112-06-3), isoxaflutole (CAS RN 141112-29-0), mesotrione (CAS RN 104206-82-8), pyrasulfotole (CAS RN 365400-11-9), pyrazolynate (CAS RN 58011-68-0), pyrazoxyfen (CAS RN 71561-11-0), sulcotrione (CAS RN 99105-77-8), tefuryltrione (CAS RN 473278-76-1), tembotrione (CAS RN 335104-84-2) and topramezone (CAS RN 210631-68-8); including, where applicable, agrochemically acceptable salts thereof.

### Methods of Use

The present invention further provides a method of selectively controlling weeds at a locus comprising crop plants and weeds, wherein the plants are obtained by any of the methods of the current invention described above, wherein the method comprises application to the locus of a weed controlling amount of one or more herbicides. Any of the plants described herein may be used within these methods of the invention. The term “locus” may include soil, seeds, and seedlings, as well as established vegetation. Herbicides can suitably be applied pre-emergence or post-emergence of the crop or weeds.

The term “weed controlling amount” is meant to include functionally, an amount of herbicide which is capable of affecting the growth or development of a given weed. Thus, the amount may be small enough to simply retard or suppress the growth or development of a given weed, or the amount may be large enough to irreversibly destroy a given weed.

Thus, the present invention provides a method of controlling weeds at a locus comprising applying to the locus a weed-controlling amount of one or more herbicides, where the locus comprises a plant that has been transformed with a nucleic acid molecule encoding the herein disclosed fungus-derived HPPD polypeptide or variant thereof that confers resistance or tolerance to HPPD herbicides, alone or in combination with one or more additional nucleic acid molecules encoding polypeptides that confer desirable traits.

In one embodiment, the present invention provides plants and methods useful for the control of unwanted plant species in crop fields, wherein the crop plants are made resistant to HPPD chemistry by transformation to express genes encoding the herein disclosed fungus-derived HPPD polypeptides, and where an HPPD herbicide is applied as an over-the-top application in amounts capable of killing or impairing the growth of unwanted plant species (weed species, or, for example, carry-over or “rogue” or “volunteer” crop plants in a field of desirable crop plants). The application may be pre- or post-emergence of the crop plants or of the unwanted species, and may be combined with the application of other herbicides to which the crop is naturally tolerant, or to which it is resistant via expression of one or more other herbicide resistance transgenes.

In another embodiment, the invention also relates to a method of protecting crop plants from herbicidal injury. In the cultivation of crop plants, especially on a commercial scale, correct crop rotation is crucially important for yield stability (the achievement of high yields of good quality over a long period) and for the economic success of an agronomic business. Use of the herbicide may cause agronomically unacceptable phytotoxic damage to the crop plants in subsequent crops (“carry-over” damage). Accordingly, the herbicide resistant or tolerant plants of the invention are also useful for planting in a locus of any short term carry-over of herbicide from a previous application (e.g., by planting a plant of the invention in the year following application of an herbicide to reduce the risk of damage from soil residues of the herbicide).

### Examples

The invention is now described with reference to the following Examples. These Examples are provided for the purpose of illustration only, and the invention is not limited to these Examples, but rather encompasses all variations which are evident as a result of the teachings provided herein.

### Isolation

A putative fungus was isolated from x0.25 YENB agar plate with chloramphenicol (50 μg/ml) and Penicillin-Streptomycin (100 units penicillin and 100 μg Streptomycin per ml).

### Gene Annotation

A first isolated fungus was shown to belong to the genus Trichoderma spp., by sequencing, several genes including 18S Internal Transcribed spacer1 (ITS1), translation elongation factor 1-alpha (tef1), endochitinase 42 (ech42) and RNA polymerase II (rpb2), followed by whole genome sequencing of the fungus. A 4-Hydroxyphenylpyruvate dioxygenase (HPPD) gene of the isolated fungus was detected by homology search (BLASTx) and cloned.

Another fungus was shown to belong to the genus Talaromyces spp., by sequencing several genes. A 4-Hydroxyphenylpyruvate dioxygenase (HPPD) gene of the isolated fungus was detected by homology search (BLASTx) and cloned.

### Bacterial HPPD-Activity Assay

The coding sequence of the identified hppd cDNA was amplified by PCR and cloned to a pET16 expression vector. E. coli BL21 (DE3) were transformed with the expression vector and monitored for HPPD activity using a colorimetric method as essentially explained in Rocaboy-Faquet. E. et al. (DOI: 10.1007/s00253-014-5793-5) with two minor modifications, namely: 1) The transformed bacteria were inoculated on an agar based medium instead of a liquid medium; 2) The screen was performed at 25° C. for 4 days.

The screen media was prepared with a concentration gradient of HPPD inhibitors at the indicated concentrations and the transformed bacteria were inoculated on the screening agar plates. HPPD activity was detected by a brown halo resulting from the HPPD-mediated conversion of p-hydroxyphenylpyruvate (HPP) into homogentisate (HGA), which later is oxidized to a melanin-like pigment. Tembotrione resistance of the transformed bacteria was compared to that of plates inoculated with E. coli BL21 (DE3) transformed with either an empty pET16 vector or a pET16 vector transformed with Arabidopsis thaliana hppd cDNA (accession no. AF047834).

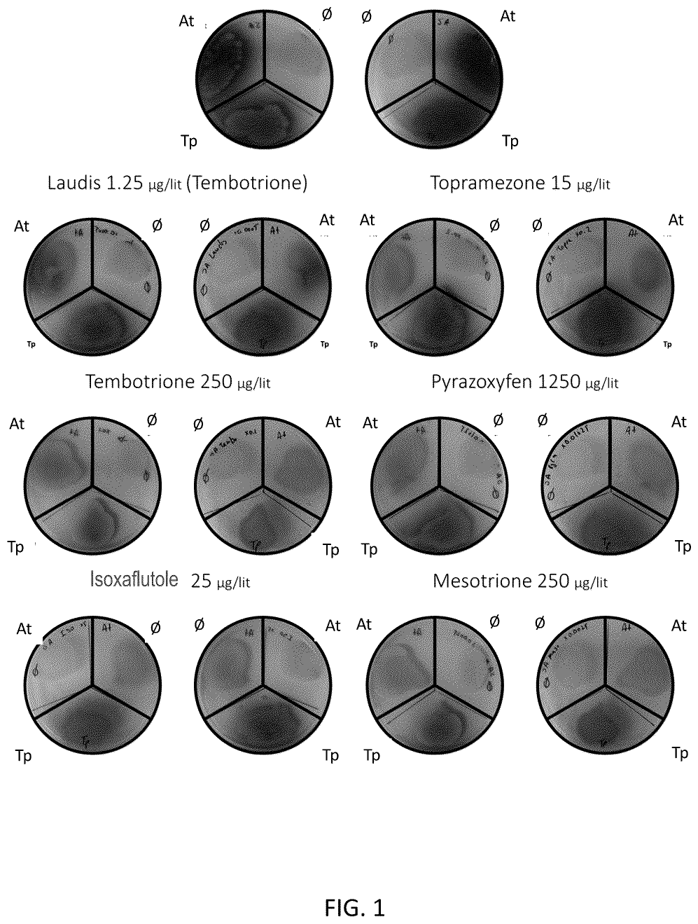

As seen in FIG. 1, transformation of E. coli BL21 (DE3) with the HPPD derived from the novel Trichoderma fungus (TP) resulted in detection of HPPD activity (brown halo) on plates with as much as to 250 μg/ml of Tembotrione, 250 μg/ml of Mesotrione, 25 μg/ml of isoxaflutole, 25 μg/ml of Topramezone and 1250 μg/ml of Pyrazoxyfen, while of E. coli BL21 (DE3) transformed with the Arabidopsis thaliana HPPD (AtHPPD) showed poor HPPD activity if at all. No activity was detected in the empty vector control plates (Ø).

In-Planta Screen of Plants Transformed with Fungal HPPD

The hppd coding region was cloned into pPA35H binary vector under the control of the CaMV35S constitutive promoter and HSP terminator and was used to transform Arabidopsis plants using agrobacterium according to the dipping flower method, as essentially described in WO 2018/178975. Tl generation transformed seeds were germinated on Basta selection (Bayer) according to manufacturer instructions. 3-4 days post germination, plants were treated with 0.1% and 0.05% Tembotrione (Laudis (Bayer)). HPPD resistant plants were detected one-week post germination.

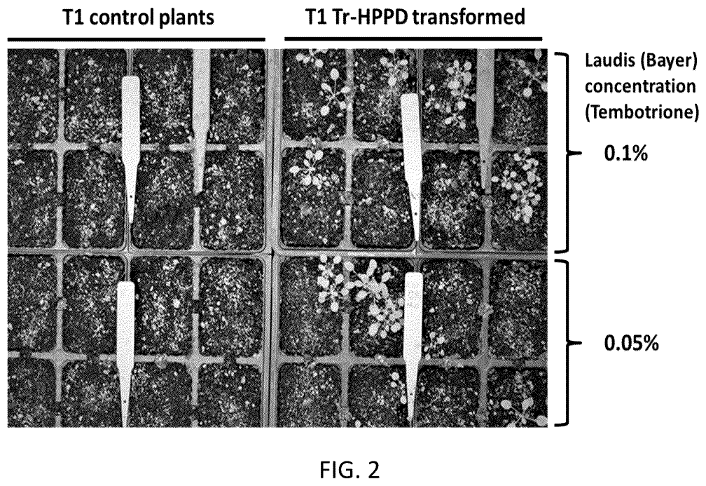

As seen from FIG. 2, transformation of Arabidopsis Tl plants with the herein disclosed Trichoderma spp-HPPD gene enabled growth in the presences of the Tembotrione herbicide, while no plant growth was observed in the control. This shows that transformation of plant to express the herein disclosed Trichoderma spp-HPPD gene enables its growth in the presence of the herbicide Tembotrione, which is highly effective in weed control of plants in particular broad leaf.

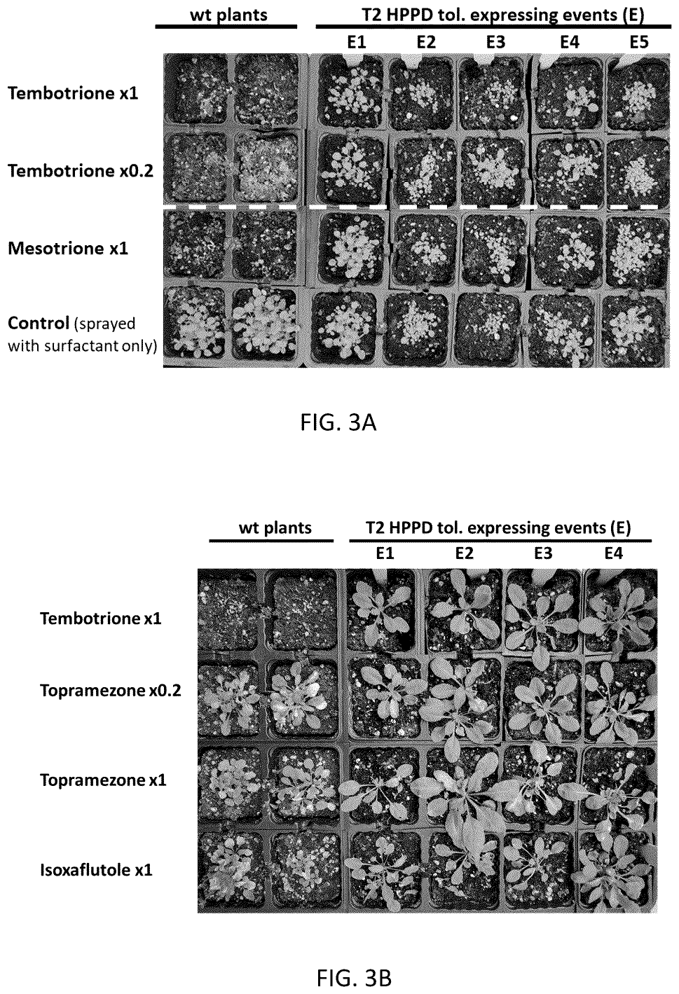

As seen from FIG. 3, T2 generation transformed seeds of Arabidopsis plants with the herein disclosed Trichoderma spp-HPPD gene enabled growth in the presences of the Tembotrione (x1=2500 μg/ml and x0.2=500 μg/ml), Mesotrione (x1=50 μg/ml), Topramezon (x1=150 μg/ml and x0.2=30 μg/ml) and Isoxaflutole (x1=50 μg/ml) herbicides, while no plant growth or an acute growth inhibition and leaf bleaching were observed in the control (wt plants). This shows that transformation of plant to express the herein disclosed Trichoderma spp-HPPD gene enables its growth in the presence of the herbicide Tembotrione, Mesotrione, Topramezon and Isoxaflutole, which are highly effective in weed control of plants, in particular broad leaf plants. This results emphasis the advantageous tolerance of plant transformed to express the herein-disclosed Trichoderma spp-HPPD gene to different HPPD inhibitors.

### Structure

Structural prediction of SEQ ID NO: 1 was performed using MODELLER (Webb B, Sali A. Curr Protoc Protein Sci. 2016 Nov. 1; 86:2.9.1-2.9.37) via the hhpred server (Zimmermann L et al. J Mol Biol. 2018 Jul. 20. S0022-2836(17)30587-9) using PDB entries 1T47, 1SP8 and IFTZ as best fit templates (48.1%, 31.7% and 33.8% seq ID respectively).

The predicted structure was fitted on the HPPD structure of A. thaliana (PDB: 5YWG) with ExPASy Swiss-Pdb Viewer (Guex, N. and Peitsch, M. C. Electrophoresis 1997 18, 2714-2723) iterative magic fit (RMS=1.01 Å).

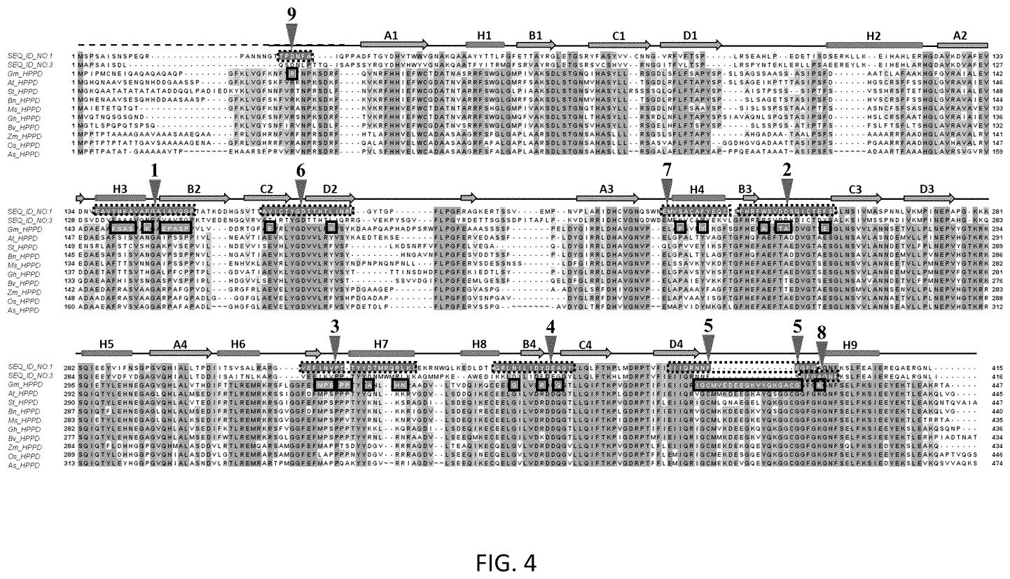

Multiple sequence alignment of SEQ ID NO: 1 with SEQ ID NO: 3 and Plant HPPD sequences of Arabidopsis thaliana (At_HPPD), Brassica napus—Canola (Bn_HPPD), Zea mays—Corn (Zm_HPPD), Solanum tuberosum—Potato (St_HPPD), Glycine max—Soy (Gm_HPPD), Medicago sativa—Alfalfa (Ms_HPPD), Gossypium hirsutum—Cotton (Gh_HPPD), Beta vulgaris—Sugar beet (Bv_HPPD) Oryza sativa—Rice (Os_HPPD) and Avena sativa—Oat (As_HPPD) was performed via T-coffee server with expresso mode (Notredame, Higgins, Heringa, JMB, 302 (205-217) 2000). The alignment was verified over the structures fit of SEQ ID NO: 1 model over the A. thaliana HPPD structure (PDB: 5YWG), as shown in FIG. 4 (The positions of secondary structural elements are shown on top of the alignment based on the structural elements of the HPPD of A. thaliana (PDB: 5YWG)). Motifs 1-9 of SEQ ID NO: 1 and/or 3 (set forth in SEQ ID NOs: 38-46 and SEQ ID NO: 47-54) are indicated by dashed-line boxes indicated by the numbered triangles (the motifs of At_HPPD, Zm_HPPD, Gm_HPPD, St_HPPD, Bn_HPPD, Ms_HPPD, Gh_HPPD, Bv_HPPD, Os_HPPD and AsHPPD are not indicated, but correspond to the motifs indicated for SEQ ID NO: 1).

Based on the 3D model nine structural motifs were recognized on SEQ ID NO: 1:
Motif 1: Glu137-Pro154 (set forth in SEQ ID NO: 38), located on helix-3 (H3) and sheet-B2 (B2).
Motif 2: Phe238-Ser254 (set forth in SEQ ID NO: 39), located on sheet-B3 (B3) and loop between sheet-B3 (B3) and sheet-C3 (C3).
Motif 3: Val318-Tyr337 (set forth in SEQ ID NO: 40), located on a short helix between helix-6 (H6) and helix-7 (H7), includes helix-7 (H7).
Motif 4: Gln352-Tyr365 (set forth in SEQ ID NO: 41), located between helix 8 (H8) and sheet C4 (C4), includes sheet B4 (B4).
Motif 5: Glu383-Gly394 (set forth in SEQ ID NO:42), located on sheet-D4 (D4) and includes loop between sheet-D4 (D4) and helix-9 (H9).
Motif 6: Ala167-Arg182 (set forth in SEQ ID NO:43), located on sheet-C2 (C2) and sheet-C3 (C3)
Motif 7: Glu224-Cys235 (set forth in SEQ ID NO:44), located on helix 7 (H7)
Motif 8: Gly392-Phe398 (set forth in SEQ ID NO:45), located on the loop between sheet-D4 (D4) and helix-9 (H9) and on helix-9 (H9)
Motif 9: Thr21-Ala26 (set forth in SEQ ID NO:46), located on unstructured region at the N-terminal.

Based on the 3D model nine structural motifs were recognized on SEQ ID NO:3:
Motif 1: Glu130-Pro147 (set forth in SEQ ID NO: 47), located on helix-3 (H3) and sheet-B2 (B2).
Motif 2: Phe239-Ser255 (set forth in SEQ ID NO: 48), located on sheet-B3 (B3) and loop between sheet-B3 (B3) and sheet-C3 (C3).
Motif 3: Val319-Lys338 (set forth in SEQ ID NO: 49), located on a short helix between helix-6 (H6) and helix-7 (H7), includes helix-7 (H7).
Motif 4: Lys351-Tyr364 (set forth in SEQ ID NO: 50), located between helix 8 (H8) and sheet C4 (C4), includes sheet B4 (B4).
Motif 5: Glu382-Gly393 (set forth in SEQ ID NO: 51), located on sheet-D4 (D4) and includes loop between sheet-D4 (D4) and helix-9 (H9).
Motif 6: Ala161-Arg176 (set forth in SEQ ID NO:52), located on sheet-C2 (C2) and sheet-C3 (C3)
Motif 7: Glu226-Val237 (set forth in SEQ ID NO:53), located on helix 7 (H7)
Motif 8: Gly392-Phe398 (set forth in SEQ ID NO:45), located on the loop between sheet-D4 (D4) and helix-9 (H9) and on helix-9 (H9)
Motif 9: Gln10-Pro15 (set forth in SEQ ID NO:54), located on unstructured region at the N-terminal.

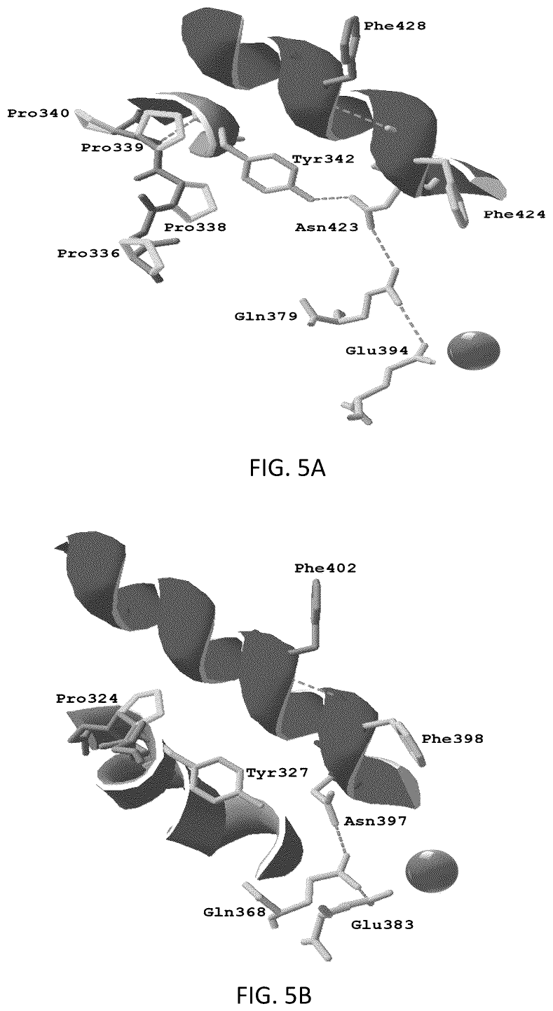

The 3D model of HPPD of A. thaliana and of that built based on SEQ ID NO: 1, can be seen in FIG. 5A and FIG. 5B, respectively.

The active site metal ion (sphere) of the A. thaliana HPPD (FIG. 5A) is linked through three hydrogen bonds to helix 7 (H7) (originally in turquoise) constructing a hydrogen bond chain. Residues of the hydrogen bond chain are originally shown in orange. The bound inhibitor (originally in pink) is stabilized through x-stacking interaction with Phe424. The spatial location of Helix 7 is affected the hydrogen bond of Tyr342 and multiple Proline residues (originally in yellow).

As seen in FIG. 4 motif 3, forming helix 7 of SEQ ID NO: 1 (SEQ ID NO: 40), has less proline residues resulting in a longer helix with low rigidity, as seen in FIG. 5B, affecting weak interaction of Tyr327 with Asn397 in the predicted structure of SEQ ID NO:1, thus destabilizing the interaction with the inhibitor.

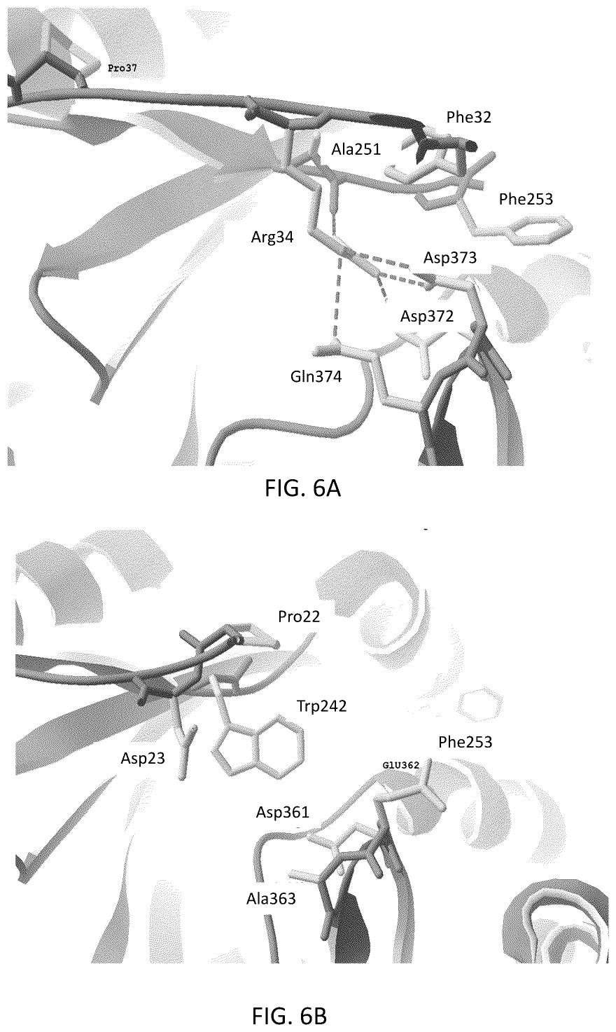

As further seen from FIG. 6A and FIG. 6B, which show a 3D model of the spatial interactions of the structural elements formed by motifs 2 and 3 of A. thaliana and of that built based on SEQ ID NO: 1, respectively.

As seen from FIG. 6A, in the HPPD of A. thaliana, Asp372, Asp373 and Gln374 (motif 4 of At_HPPD in FIG. 3) forms hydrogen bonds with Arg34 of the N-terminal loop and Ala251 of motif 2 of At_HPPD. The location of the N-terminal loop is further stabilized through x-stacking interaction of Phe253 and Phe32.

As seen from FIG. 6B, in the predicted SEQ ID NO:1 structure, Trp242 of motif 2 (SEQ ID NO: 39) replaces Ala251, and forms a steric disturbance preventing hydrogen bonds between the motifs. Stabilizing through x-stacking was also not detected.

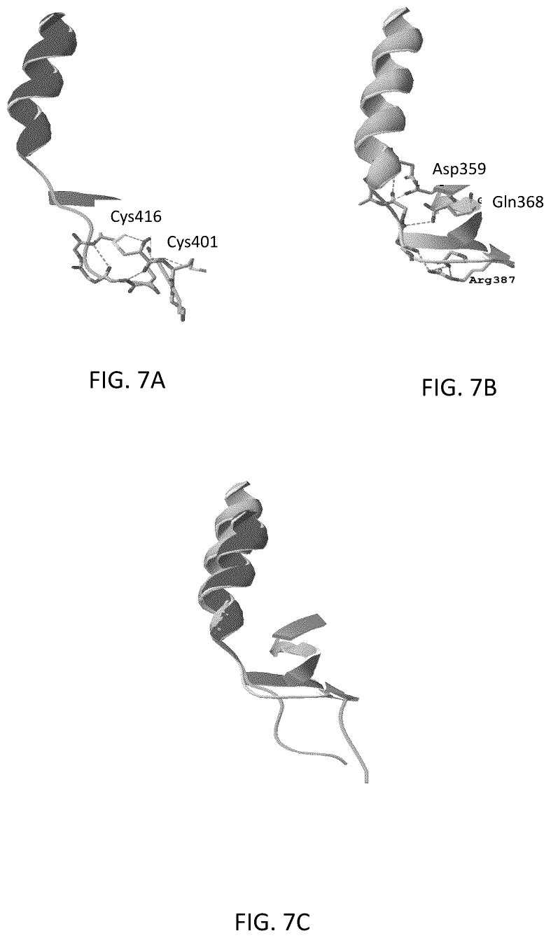

As further seen from FIG. 7A and FIG. 7B, which show the influence of the differences in motif 5 on the loop and helix 9 (H9) of HPPD of A. thaliana and of that built based on SEQ ID NO: 1 (SEQ ID NO: 24), respectively. As seen from FIG. 7A, motif 5 of At_HPPD forms a long loop tightened by an S—S bond formed by Cys401 and Cys416. In the predicted structure of the HPPD, shown in FIG. 7B, no such S—S bond is formed. Instead, a short loop (in orange) is formed and bound by hydrogen bonds to Asp359 and Gln368 of motif 4 (SEQ ID NO: 41) (secondary structure elements in gray). FIG. 7C, shows the superimposition of FIG. 7A and FIG. 7B.

### Generation of cTP Chimeric HPPDs

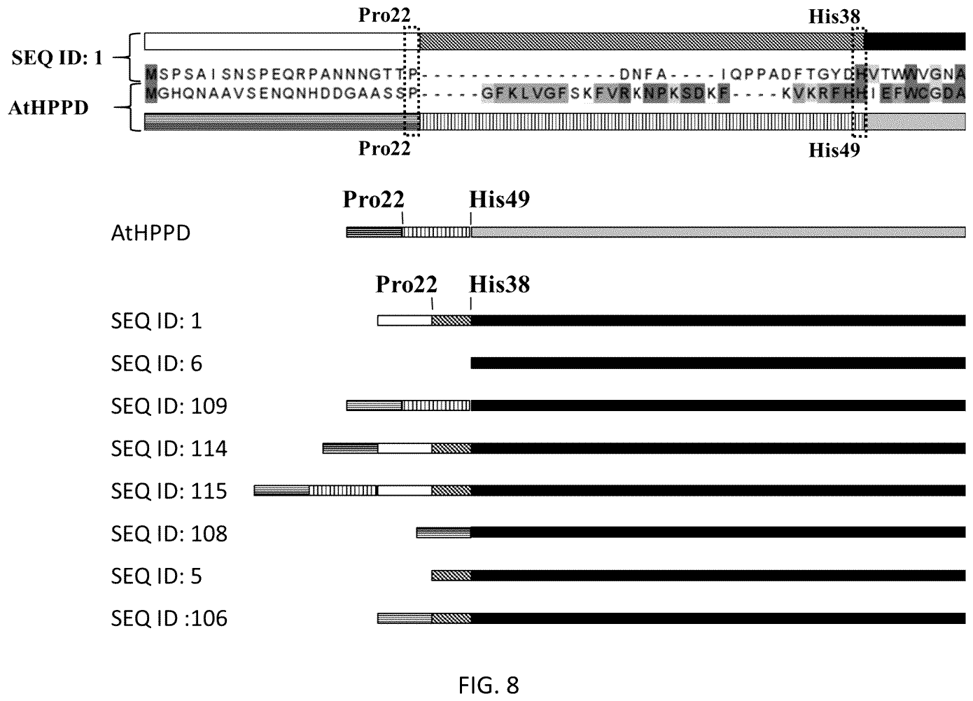

The N-terminal sequence of the HPPD gene of the isolated Trichoderma fungus (SEQ ID NO: 1) was aligned over several plant N-terminal chloroplast transit peptide (cTP) sequences. Several chimeric constructs were designed with variations of deletions at the N-terminal region of SEQ ID NO: 1 (deletion of amino acids 1-22 (422) (SEQ ID NO: 5) or of amino acids 1-38 (438) (SEQ ID NO: 6)). The HPPD N-terminal peptides of Arabidopsis thaliana (amino acids 1-22 (SEQ ID NO: 104) or amino acids 1-49 (SEQ ID NO: 105) comprising the cTP of Arabidopsis thaliana) were added to SEQ ID NO: 1 enzyme as well as to the N-terminal truncated versions of SEQ ID NO: 1 (i.e. SEQ ID NO: 5 and SEQ ID NO: 6) as illustrated in FIG. 8.

### HPPD-Activity Screen of cTP Chimeric HPPDs

The cDNA sequences of the cTP chimeric HPPD set forth in FIG. 8 were amplified by PCR and cloned to a pET16 expression vector. E. coli BL21 (DE3) were transformed with the expression vector and monitored for HPPD activity using a colorimetric method as essentially explained in Rocaboy-Faquet. E. et al. (DOI: 10.1007/s00253-014-5793-5) with two minor modifications, namely: 1) The transformed bacteria were inoculated on an agar based medium instead of a liquid medium; 2) The screen was performed at 25° C. for 4 days.

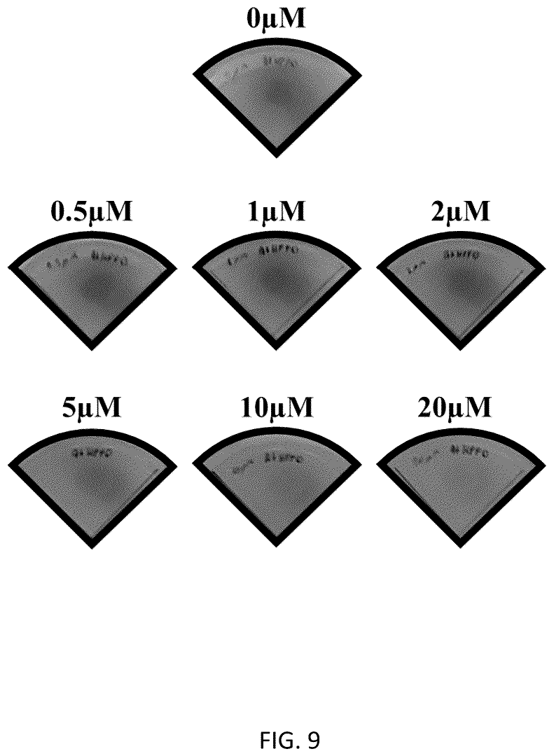

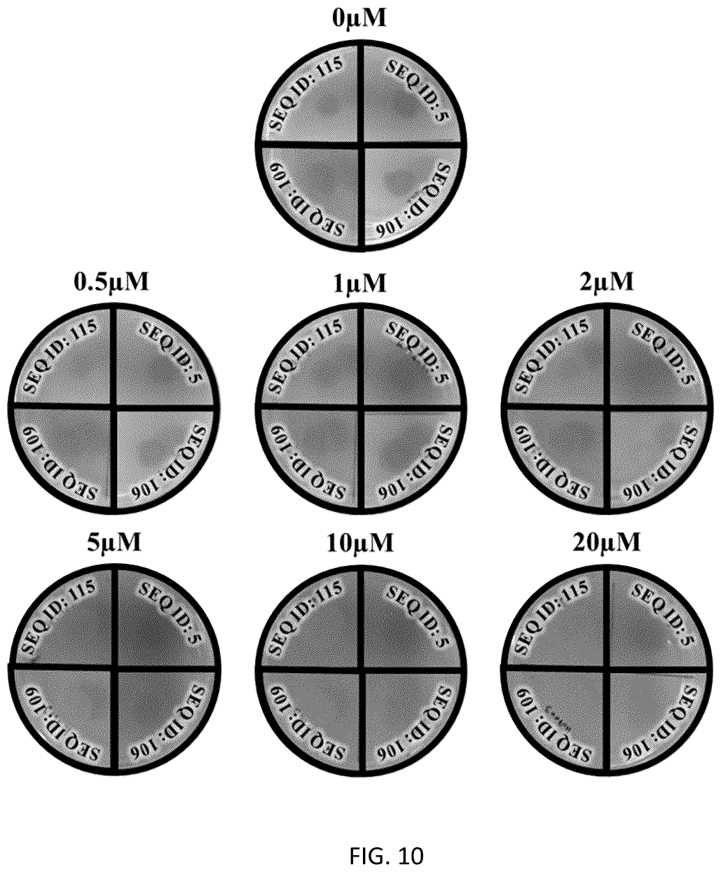

The screen media was prepared with a concentration gradient of Laudis herbicide at the indicated concentrations of the active ingredient Tembotrione and the transformed bacteria were inoculated on the screening agar plates. HPPD activity was detected by a brown halo resulting from the HPPD-mediated conversion of p-hydroxyphenylpyruvate (HPP) into homogentisate (HGA), which later is oxidized to a melanin-like pigment. Tembotrione resistance of the transformed bacteria FIG. 9, was compared to that of plates inoculated with E. coli BL21 (DE3) transformed with Arabidopsis thaliana hppd cDNA (accession no. AF047834) FIG. 10.

Constructs SEQ ID NO: 6 (HPPD derived from Trichoderma fungus, truncated of amino acids 1-38 (A38)), SEQ ID NO: 114 (HPPD derived from Trichoderma fungus and with the addition of amino acids 1-22 of Arabidopsis thaliana) and SEQ ID NO: 108 (HPPD derived from Trichoderma fungus, truncated of amino acids 1-38 (438) and with the addition of amino acids 1-22 of Arabidopsis thaliana) lost their HPPD enzymatic activity (results not shown).

As seen from FIG. 9, SEQ ID NO: 109 (HPPD derived from Trichoderma fungus, truncated of amino acids 1-38 (438) and with the addition of amino acids 1-49 of Arabidopsis thaliana) and SEQ ID NO: 115 (HPPD derived from Trichoderma fungus and with the addition of amino acids 1-49 of Arabidopsis thaliana) were inhibited at tembotrione concentration above 2 μM and 5 μM, respectively, similarly to the endogenous AtHPPD of Arabidopsis thaliana—see FIG. 10.

However, surprisingly, the enzyme activity of SEQ ID NO: 5 (HPPD derived from Trichoderma fungus, truncated of amino acids 1-22 (422)) and SEQ ID NO: 106 (HPPD derived from Trichoderma fungus in which amino acids 1-22 (A22) of the fungal HPPD were substituted with amino acids 1-22 of Arabidopsis thaliana) was maintained at concentrations as high as to 20 μM. This indicates that amino acids 1-22 (422) of the fungal HPPD have minor if any importance to its enzymatic activity and may be substituted with a plant cTP to confer transport to chloroplasts.

### HPPD-Activity Screen of Soy HPPD Mutants

The cDNA sequences of the below listed G. max HPPD (SEQ ID NO: 118) and G. max HPPD mutants were amplified by PCR and cloned to a pET16 expression vector. E. coli BL21 (DE3) were transformed with the expression vectors and monitored for HPPD activity using a colorimetric method as essentially explained in Rocaboy-Faquet. E. et al. (DOI: 10.1007/s00253-014-5793-5) with two minor modifications, namely: 1) The transformed bacteria were inoculated on an agar based medium instead of a liquid medium; 2) The screen was performed at 25° C. for 4 days, in the absence or presence of increasing concentrations of tembotrione (Laudis) HPPD inhibitor. A brown halo is indicative of HPPD activity
GmHPPD SEQ ID NO: 118 (MPIPCNEIQAQAQAQAQPGFKLVGFKNFVRTNPKSDRFQVNRFHHIEFW CTDATNASRRFSWGLGMPIVAKSDLSTGNQIHASYLLRSGDLSFLFSAPYS PSLSAGSSAASSASIPSFDAATCLAFAAKHGFGVRAIALEVADAEAAFSAS VAKGAEPASPPVLVDDRTGFAEVRLYGDVVLRYVSYKDAAPQAPHADPS RWFLPGFEAAASSSSFPELDYGIRRLDHAVGNVPELAPAVRYLKGFSGFH EFAEFTAEDVGTSESGLNSVVLANNSETVLLPLNEPVYGTKRKSQIETYLE HNEGAGVQHLALVTHDIFTTLREMRKRSFLGGFEFMPSPPPTYYANLHNR AADVLTVDQIKQCEELGILVDRDDQGTLLQIFTKPVGDRPTIFIXIIQRIGC MVEDEEGKVYQKGACGGFGKGNFSELFKSIEEYEKTLEAKRTA), the native G. max HPPD (Accession number: ABQ96868)
GmHPPD.1=GmHPPD (SEQ ID NO: 118) mutated to substitute its motif 1 with motif 1 of SEQ ID NO: 1—i.e. to include amino acid sequences SEQ ID NO: 9-10 or 38;
GmHPPD.2=GmHPPD (SEQ ID NO: 118) mutated to substitute its motif 6.2 (SEQ ID NO: 68) first Y with motif 6.2 of SEQ ID NO: 1 (SEQ ID NO: 20)—i.e. to include the mutation Tyr185Phe to include SEQ ID NO: 157;
GmHPPD.4=GmHPPD (SEQ ID NO: 118) mutated to substitute its motif 2.1 (SEQ ID NO: 57) with motif 2.1 of SEQ ID NO: 1 (SEQ ID NO: 11)—i.e. to include the mutation Ala254Trp to include SEQ ID NO: 129.

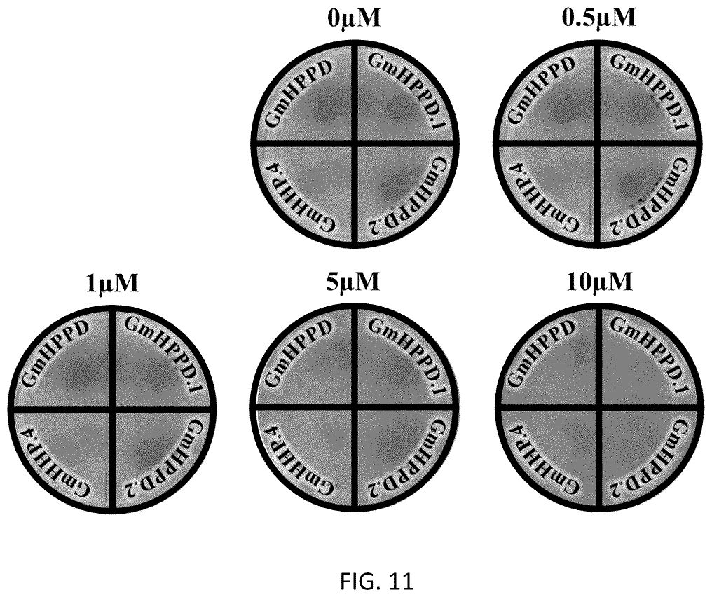

As seen from FIG. 11, tembotrione effects the native soy HPPD (GmHPPD) activity already at concentrations above 1 μM, and only minor enzymatic activity was detected at inhibitor concentration of 5 μM, and essentially no enzymatic activity was observed at 10 μM tembotrione.

However, GmHPPD.1 and GmHPPD.2 showed significant activity at 5 μM tembotrione and some activity was still observed at 10 μM tembotrione.

These results indicate that soy HPPD having its motif 1 or motif's 6.2 first Tyr substituted with motif 1 and motif's 6.2 first Trp of Trichoderma HPPD maintained activity even at high concentrations of tembotrione.

### In-Planta Screen of Soy HPPD Mutants

Tl generation transformed seeds of Camelina plants with the Soy HPPD (GmHPPD) mutated gene as compared to wt. Plant were treated with Laudis (Tembotrione) 0.075% three days post seeding. EGFP transformed plants were used as control. Images taken 19 days post treatment. The following were tested:
GmHPPD—SEQ ID NO: 118 the native G. max HPPD (Accession number: ABQ96868)
GmHPPD.1=GmHPPD (SEQ ID NO: 118) mutated to substitute its motif 1 with motif 1 of SEQ ID NO: 1—i.e. to include amino acid sequences set forth in SEQ ID NO: 9-10 or 38;
GmHPPD.2=GmHPPD (SEQ ID NO: 118) mutated to substitute its motif 6.2 (SEQ ID NO: 68) first Y with motif 6.2 of SEQ ID NO: 1 (SEQ ID NO: 20)—i.e. to include the mutation Tyr185Phe to include SEQ ID NO: 157;
GmHPPD.3=GmHPPD (SEQ ID NO: 118) mutated to substitute its motif 7.2 (SEQ ID NO: 70) first Y with motif 7.2 of SEQ ID NO: 1 (SEQ ID NO: 22)—i.e. to include the mutation Tyr243Phe;
GmHPPD.4=GmHPPD (SEQ ID NO: 118) mutated to substitute its motif 2.1 (SEQ ID NO: 57) with motif 2.1 of SEQ ID NO: 1 (SEQ ID NO: 11)—i.e. to include the mutation Ala254Trp to include SEQ ID NO: 129;
GmHPPD.5=GmHPPD (SEQ ID NO: 118) mutated to substitute its motif 3.1 with motif 3.1 of SEQ ID NO: 1—i.e. to include the replacement mutations 338-MPSPPP-343 to INVPG.

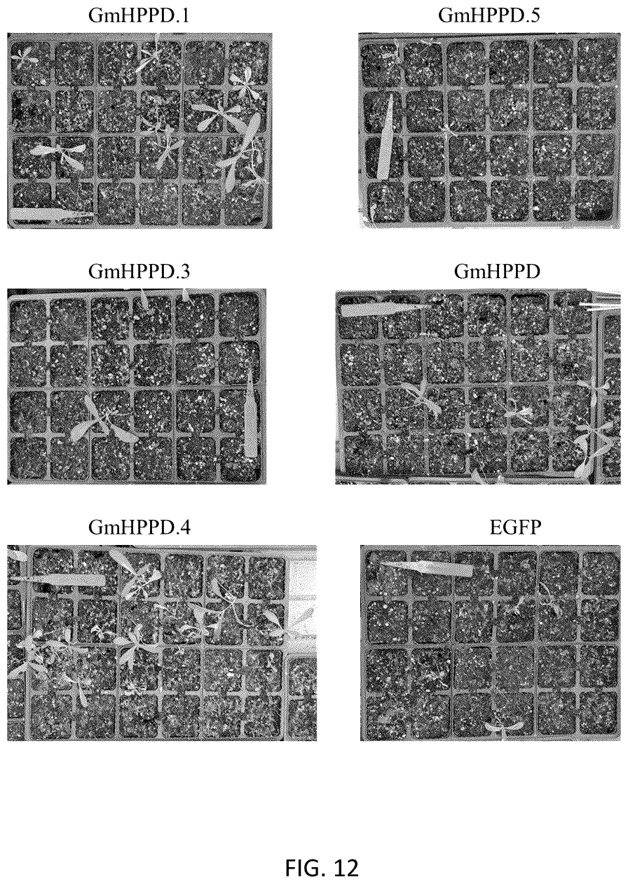

As seen from FIG. 12, Control (EGFP), WT GmHPPD and GmHPPD.5 plants were highly damaged with the recognizable bleaching effects of the herbicide.

However, for GmHPPD.1 and GmHPPD.4 seeds, numerous tolerant plants were generated, clearly indicating that these HPPD mutants are herbicide resistant. The GmHPPD.3 mutant although producing a single plant only its recovery from the treatment was pronounced.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

1. A transgenic crop plant expressing an exogenous fungal HPPD enzyme having any of the amino acid sequences set forth in SEQ ID NO: 1, SEQ ID NO: 3, or SEQ ID NO: 5, wherein the exogenous fungal HPPD enzyme catalyzes conversion of phydroxyphenylpyruvate (HPP) into homogentisate (HGA) in the plant, and wherein the fungal HPPD enzyme has less than 50% amino acid sequence similarity to an endogenous HPPD enzyme of the plant.

2. The crop plant of claim 1, wherein the polypeptide further comprises a plant chloroplast transit peptide.

3. The crop plant of claim 1, being selected from the group consisting of: soybean, cotton, maize, wheat, rice, barley, cowpea, chickpea, sorghum, beans, canola, alfalfa, flax, sunflower, safflower, millet, rye, sugarcane, sugar beet, cocoa, tea, Brassica, cotton, camelina, coffee, sweet potato, flax, peanut, clover; lettuce, tomato, cucurbits, cassava, potato, carrot, radish, pea, lentils, cabbage, cauliflower, broccoli, Brussels sprouts, peppers, pineapple, citrus, apples, pears, peaches, apricots, walnuts, avocado, banana, palm, eucalyptus, poplar, pine, coconut, orchids, petunia, carnations, roses, switchgrass, prairie grasses, indiangrass, and big bluestem grass.

4. The crop plant of claim 3, being a soybean plant or cotton plant.

5. The crop plant of claim 1, being a dicot plant.

6. The crop plant of claim 1, being a monocot plant.

7. The crop plant of claim 1, comprising a nucleic acid molecule encoding the exogenous fungal HPPD enzyme.

8. The crop plant of claim 7, wherein the nucleic acid molecule has the sequence set forth in SEQ ID NO: 2 or SEQ ID NO: 4.

9. The crop plant of claim 1 being resistant to post-emergence application of an HPPD inhibitor at a concentration sufficient to kill a plant that does not express the exogenous fungal HPPD enzyme.

10. A seed of the plant of claim 1.
